(19) United States
(12) Patent Application Publication
(10) Pub. No.: US 2020/0147059 A1
(43) Pub. Date: May 14, 2020

(54) NEW THERAPEUTIC USES OF ENZYME INHIBITORS

(71) Applicant: Proximagen, LLC, Plymouth, MN (US)

(72) Inventor: William PULLMAN, Plymouth, MN (US)

(73) Assignee: Proximagen, LLC, Plymouth, MN (US)

(21) Appl. No.: 16/620,373

(22) PCT Filed: Jun. 8, 2018
(86) PCT No.: PCT/GB2018/051558

§ 371 Date: Dec. 6, 2019

(30) Foreign Application Priority Data
Jun. 8, 2017 (GB) 1709136.4

(51) Int. Cl. A61K 31/437; A61K 31/15; A61K 31/198; A61K 31/165

(52) U.S. Cl. CPC A61K 31/437; A61K 31/165; A61K 31/198; A61K 31/15

(57) ABSTRACT

Inhibitors of VAP-1/SSAO activity, and pharmaceutical compositions comprising the same, are useful for the prevention and/or treatment of migraine, including headache, chronic migraine, episodic migraine, medication overuse headache disorder (MOU), migraine without aura, migraine with aura, migraine aura without headache, ocular migraine, vestibular migraine, basilar migraine, hemiplegic migraine, ophthalmoplegic migraine, and tension-type headache (TTH).

### FIELD OF THE INVENTION

This invention relates to the use of inhibitors of VAP-1/SSAO activity, and pharmaceutical compositions comprising the same, for the prevention and/or treatment of migraine, which includes the prevention and/or treatment of headache, chronic migraine; episodic migraine; medication overuse headache disorder (MOU); migraine without aura; migraine with aura; migraine aura without headache; ocular migraine; vestibular migraine; basilar migraine; hemiplegic migraine; ophthalmoplegic migraine; and tension-type headache (TTH).

### BACKGROUND ART

Semicarbazide-sensitive amine oxidase (SSAO), otherwise known as Vascular Adhesion Protein-1 (VAP-1) or Amine Oxidase, Copper Containing 3 (AOC3), belongs to the copper-containing amine oxidase family of enzymes (EC.1.4.3.6). Members of this enzyme family are sensitive to inhibition by semicarbazide and utilize cupric ion and protein-derived topaquinone (TPQ) cofactor in the oxidative deamination of primary amines to aldehydes, hydrogen peroxide, and ammonia according to the following reaction:

R—CH₂—NH₂+O₂→R—CHO+H₂O₂+NH₃

Known substrates for human SSAO include endogenous methylamine and aminoacetone as well as some xenobiotic amines such as benzylamine [Lyles, Int. J. Biochem. Cell Biol. 1996, 28, 259-274; Klinman, Biochim. Biophys. Acta 2003, 1647(1-2), 131-137; Mátyus et al., Curr. Med. Chem. 2004, 11(10), 1285-1298; O'Sullivan et al., Neurotoxicology 2004, 25(1-2), 303-315]. In analogy with other copper-containing amine oxidases, DNA-sequence analysis and structure determination suggest that the tissue-bound human SSAO is a homodimeric glycoprotein consisting of two 90-100 kDa subunits anchored to the plasma membrane by a single N-terminal membrane spanning domain [Morris et al., J. Biol. Chem. 1997, 272, 9388-9392; Smith et al., J. Exp. Med. 1998, 188, 17-27; Airenne et al., Protein Science 2005, 14, 1964-1974; Jakobsson et al., Acta Crystallogr. D Biol. Crystallogr. 2005, 61(Pt 11), 1550-1562].

SSAO activity has been found in a variety of tissues including vascular and non-vascular smooth muscle tissue, endothelium, and adipose tissue [Lewinsohn, Braz. J. Med. Biol. Res. 1984, 17, 223-256; Nakos & Gossrau, Folia Histochem. Cytobiol. 1994, 32, 3-10; Yu et al., Biochem. Pharmacol. 1994, 47, 1055-1059; Castillo et al., Neurochem. Int. 1998, 33, 415-423; Lyles & Pino, J. Neural. Transm. Suppl. 1998, 52, 239-250; Jaakkola et al., Am. J. Pathol. 1999, 155, 1953-1965; Morin et al., J. Pharmacol. Exp. Ther. 2001, 297, 563-572; Salmi & Jalkanen, Trends Immunol. 2001, 22, 211-216]. In addition, SSAO protein is found in blood plasma and this soluble form appears to have similar properties as the tissue-bound form [Yu et al., Biochem. Pharmacol. 1994, 47, 1055-1059; Kurkijarvi et al., J. Immunol. 1998, 161, 1549-1557]. It has recently been shown that circulating human and rodent SSAO originates from the tissue-bound form [Goktirk et al., Am. J. Pathol. 2003, 163(5), 1921-1928; Abella et al., Diabetologia 2004, 47(3), 429-438; Stolen et al., Circ. Res. 2004, 95(1), 50-57], whereas in other mammals the plasma/serum SSAO is also encoded by a separate gene called AOC4 [Schwelberger, J. Neural. Transm. 2007, 114(6), 757-762].

The precise physiological role of this abundant enzyme has yet to be fully determined, but it appears that SSAO and its reaction products may have several functions in cell signalling and regulation. For example, recent findings suggest that SSAO plays a role in both GLUT4-mediated glucose uptake [Enrique-Tarancon et al., J. Biol. Chem. 1998, 273, 8025-8032; Morin et al., J. Pharmacol. Exp. Ther. 2001, 297, 563-572] and adipocyte differentiation [Fontana et al., Biochem. J. 2001, 356, 769-777; Mercier et al., Biochem. J. 2001, 358, 335-342]. In addition, SSAO has been shown to be involved in inflammatory processes where it acts as an adhesion protein for leukocytes [Salmi & Jalkanen, Trends Immunol. 2001, 22, 211-216; Salmi & Jalkanen, in “Adhesion Molecules: Functions and Inhibition” K. Ley (Ed.), 2007, pp. 237-251], and might also play a role in connective tissue matrix development and maintenance [Langford et al., Cardiovasc. Toxicol. 2002, 2(2), 141-150; Goktirk et al., Am. J. Pathol. 2003, 163(5), 1921-1928]. Moreover, a link between SSAO and angiogenesis has recently been discovered [Noda et al., FASEB J. 2008, 22(8), 2928-2935], and based on this link it is expected that inhibitors of SSAO have an anti-angiogenic effect.

Several studies in humans have demonstrated that SSAO activity in blood plasma is elevated in conditions such as congestive heart failure, diabetes mellitus, Alzheimer's disease, and inflammation [Lewinsohn, Braz. J. Med. Biol. Res. 1984, 17, 223-256; Boomsma et al., Cardiovasc. Res. 1997, 33, 387-391; Ekblom, Pharmacol. Res. 1998, 37, 87-92; Kurkijarvi et al., J. Immunol. 1998, 161, 1549-1557; Boomsma et al., Diabetologia 1999, 42, 233-237; Meszaros et al., Eur. J. Drug Metab. Pharmacokinet. 1999, 24, 299-302; Yu et al., Biochim. Biophys. Acta 2003, 1647(1-2), 193-199; Matyus et al., Curr. Med. Chem. 2004, 11(10), 1285-1298; O'Sullivan et al., Neurotoxicology 2004, 25(1-2), 303-315; del Mar Hernandez et al., Neurosci. Lett. 2005, 384(1-2), 183-187]. The mechanisms underlying these alterations of enzyme activity are not clear. It has been suggested that reactive aldehydes and hydrogen peroxide produced by endogenous amine oxidases contribute to the progression of cardiovascular diseases, diabetic complications and Alzheimer's disease [Callingham et al., Prog. Brain Res. 1995, 106, 305-321; Ekblom, Pharmacol. Res. 1998, 37, 87-92; Yu et al., Biochim. Biophys. Acta 2003, 1647(1-2), 193-199; Jiang et al., Neuropathol Appl Neurobiol. 2008, 34(2), 194-204]. Furthermore, the enzymatic activity of SSAO is involved in the leukocyte extravasation process at sites of inflammation where SSAO has been shown to be strongly expressed on the vascular endothelium [Salmi et al., Immunity 2001, 14(3), 265-276; Salmi & Jalkanen, in “Adhesion Molecules: Functions and Inhibition” K. Ley (Ed.), 2007, pp. 237-251]. Accordingly, inhibition of SSAO has been suggested to have a therapeutic value in the prevention of diabetic complications and in inflammatory diseases [Ekblom, Pharmacol. Res. 1998, 37, 87-92; Salmi et al., Immunity 2001, 14(3), 265-276; Salter-Cid et al., J. Pharmacol. Exp. Ther. 2005, 315(2), 553-562].

WO2007/146188 teaches that blocking SSAO activity inhibits leucocyte recruitment, reduces the inflammatory response, and is expected to be beneficial in prevention and treatment of seizures, for example, in epilepsy.

O'Rourke et al (J Neural Transm. 2007; 114(6):845-9) examined the potential of SSAO inhibitors in neurological diseases, having previously demonstrated the efficacy of SSAO inhibition in a rat model of stroke. An SSAO inhibitor is tested on relapsing-remitting experimental autoimmune encephalomyelitis (EAE), a mouse model that shares many characteristics with human multiple sclerosis. The data demonstrates the potential clinical benefit of small molecule anti-SSAO therapy in this model and therefore in treatment of human multiple sclerosis.

SSAO knockout animals are phenotypically overtly normal but exhibit a marked decrease in the inflammatory responses evoked in response to various inflammatory stimuli [Stolen et al., Immunity 2005, 22(1), 105-115]. In addition, antagonism of its function in wild type animals in multiple animal models of human disease (e.g. carrageenan-induced paw inflammation, oxazolone-induced colitis, lipopolysaccharide-induced lung inflammation, collagen-induced arthritis, endotoxin-induced uveitis) by the use of antibodies and/or small molecules has been shown to be protective in decreasing the leukocyte infiltration, reducing the severity of the disease phenotype and reducing levels of inflammatory cytokines and chemokines [Kirton et al., Eur. J. Immunol. 2005, 35(11), 3119-3130; Salter-Cid et al., J. Pharmacol. Exp. Ther. 2005, 315(2), 553-562; McDonald et al., Annual Reports in Medicinal Chemistry 2007, 42, 229-243; Salmi & Jalkanen, in “Adhesion Molecules: Functions and Inhibition” K. Ley (Ed.), 2007, pp. 237-251; Noda et al., FASEB J. 2008 22(4), 1094-1103; Noda et al., FASEB J. 2008, 22(8), 2928-2935]. This anti-inflammatory protection seems to be afforded across a wide range of inflammatory models all with independent causative mechanisms, rather than being restricted to one particular disease or disease model. This would suggest that SSAO may be a key nodal point for the regulation of the inflammatory response, and it seems therefore likely that SSAO inhibitors may be effective anti-inflammatory drugs in a wide range of human diseases.

Fibrosis can result from chronic tissue inflammation when the resolution of the inflammation is partly abrogated by the chronic nature of the inflammatory stimulus. The result can be inappropriate repair of the tissue with excessive extracellular matrix deposition (including collagen) with tissue scarring. This is a consequence of myofibroblast activation by stimuli including fibronectin and reactive oxygen species as well as growth factors such as transforming growth factor-ß-1 (TGFß-1), insulin-like growth factor-I (IGF-I), platelet-derived growth factor (PDGF) and connective tissue growth factor (CTGF) resulting in increased production of collagen, elastin, hyaluronan, glycoproteins and proteoglycans. In addition the activity of invading macrophages plays a crucial part in regulating the repair and fibrotic processes.

VAP-1 has also been implicated in the progression and maintenance of fibrotic diseases especially in the liver. Weston and Adams (J Neural Transm. 2011, 118(7), 1055-64) have summarised the experimental data implicating VAP-1 in liver fibrosis. Weston et al (EASL Poster 2010) showed highly increased expression of VAP-1 in human fibrotic liver, particularly associated with the activated myofibroblasts and collagen fibrils. This anatomical association with fibrosis was consistent with the observation that blockade of VAP-1 accelerated the resolution of carbon tetrachloride induced fibrosis, and suggested a role for the VAP-1/SSAO enzyme product H₂O₂ in the activation of the myofibroblasts. The same authors also showed that the pro-fibrotic growth factor TGFβ increased the expression of VAP-1 in liver cells by approximately 50-fold. In addition VAP-1 has been implicated in inflammation of the lung (e.g. Singh et al., 2003, Virchows Arch 442:491-495) suggesting that VAP-1 blockers would reduce lung inflammation and thus be of benefit to the treatment of cystic fibrosis by treating both the pro-fibrotic and pro-inflammatory aspects of the disease.

SSAO (VAP-1) is up regulated in gastric cancer and has been identified in the tumour vasculature of human melanoma, hepatoma and head and neck tumours (Yoong K F, McNab G, Hubscher S G, Adams D H. (1998), J Immunol 160, 3978-88; Irjala H, Salmi M, Alanen K, Gre'nman R, Jalkanen S (2001), Immunol. 166, 6937-6943; Forster-Horvath C, Dome B, Paku S, et al. (2004), Melanoma Res. 14, 135-40). One report (Marttila-lchihara F, Castermans K, Auvinen K, Oude Egbrink M G, Jalkanen S, Griffioen A W, Salmi M. (2010), J.

Immunol. 184, 3164-3173) has shown that mice bearing enzymically inactive VAP-1 grow melanomas more slowly, and have reduced tumour blood vessel number and diameter. The reduced growth of these tumours was also reflected in the reduced (by 60-70%) infiltration of myeloid suppressor cells. Encouragingly VAP-1 deficiency had no effect on vessel or lymph formation in normal tissue.

For the above reasons, it is expected that inhibition of SSAO will reduce the levels of pro-inflammatory enzyme products (aldehydes, hydrogen peroxide and ammonia) whilst also decreasing the adhesive capacity of immune cells and correspondingly their activation and final extra-vasation. Diseases where such an activity is expected to be therapeutically beneficial include all diseases where immune cells play a prominent role in the initiation, maintenance or resolution of the pathology, such inflammatory diseases and immune/autoimmune diseases. Examples of such diseases include multiple sclerosis, arthritis and vasculitis.

### SUMMARY OF THE INVENTION

According to a first aspect of the invention, the applicants have found that compounds having VAP-1 inhibitory activity are surprisingly effective in the prevention and/or treatment of migraine, wherein the prevention and/or treatment of migraine includes headache, chronic migraine; episodic migraine; medication overuse headache disorder (MOU); migraine without aura; migraine with aura; migraine aura without headache; ocular migraine; vestibular migraine; basilar migraine; hemiplegic migraine; ophthalmoplegic migraine; and tension-type headache (TTH).

VAP-1 inhibitors that are particularly useful in the prevention and/or treatment of migraine are compounds defined by formulae (I), (II), (III), and (IIIa), together with other compounds (such as (S)-carbidopa), as set out below.

### BRIEF DESCRIPTION OF THE FIGURES

Embodiments of the invention are described below, with reference to the accompanying drawings in which:

FIG. 1 shows that Sumatriptan-treated, but not saline treated, rats developed generalized allodynia during minipump infusion measured in the periorbital and hindpaw regions (FIG. 1; A, C; day 6). Mechanical thresholds returned to baseline on days 10 and 19. Saline-treated animals do not show any allodynia following the exposure to bright light stress (BLS). In contrast, sumatriptan-primed animals treated with vehicle developed time-dependent mechanical allodynia following the exposure to BLS (FIG. 1; B, D). 4-{5-[3-(4-Fluorophenyl)-3H-imidazo[4,5-c]pyridin-2-yl]pyridin-2-yl}morpholine (referred to as Compound 4) significantly reduced stress-induced periorbital and hindpaw allodynia

FIG. 2 shows the effects of LJP1207 on a CFA-induced arthritis model, which is a well-established pain model;

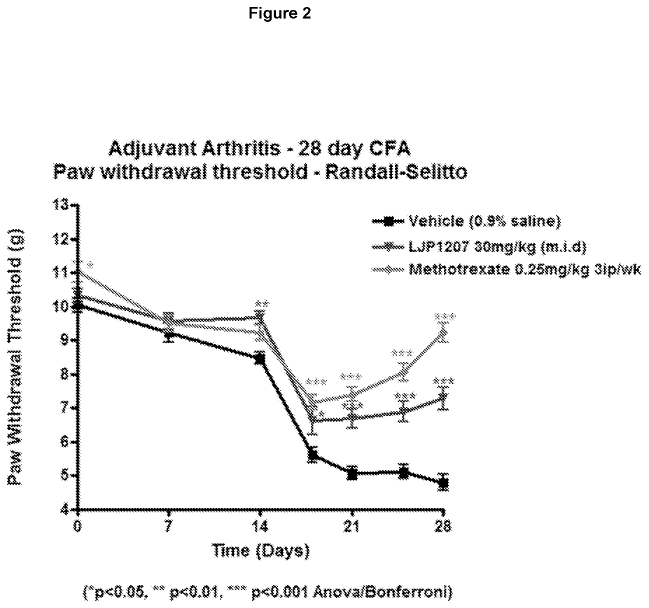

FIG. 3 shows the effects of (S)-carbidopa on CFA induced hyperalgesia in the rat at one hour and three hours post dose (left to right—vehicle; 3 mg/kg (S)-carbidopa; 10 mg/kg (S)-carbidopa; 30 mg/kg (S)-carbidopa; 100 mg/kg (S)-carbidopa; 10 mg/kg indomethacin); and

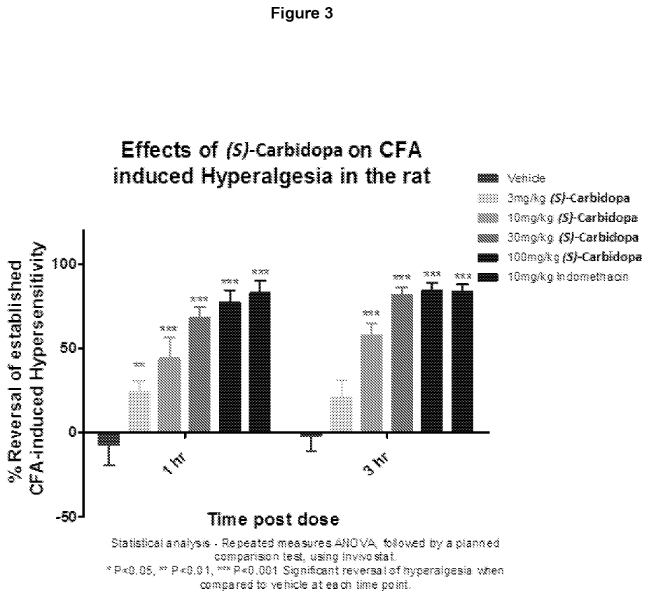

FIG. 4 shows the effects of (S)-carbidopa on paw oedema in CFA-induced hyperalgesia in the rat at 3 hours hour post dose (left to right—vehicle/vehicle; 3 mg/kg (S)-carbidopa/vehicle; 10 mg/kg (S)-carbidopa/vehicle; 30 mg/kg (S)-carbidopa/vehicle; 100 mg/kg (S)-carbidopa/vehicle; 10 mg/kg (S)-indomethacin/vehicle).

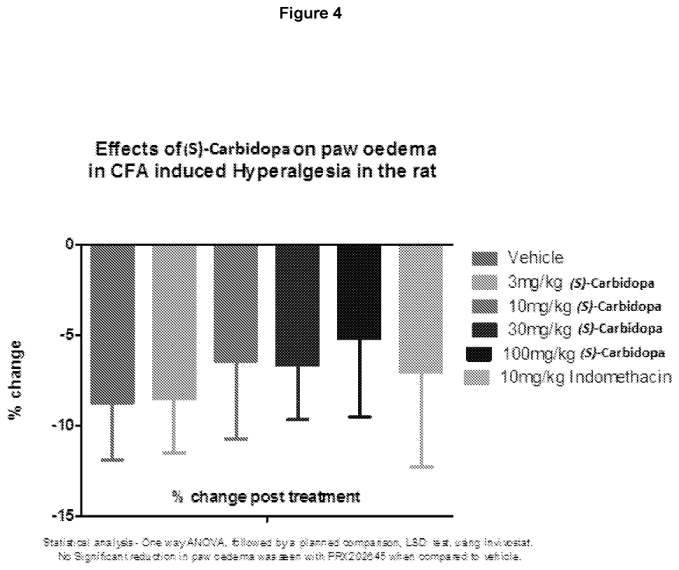

FIG. 5 shows the effect of 1-(4-{5-[3-(4-Fluorophenyl)-3H-imidazo[4,5-c]pyridin-2-yl]pyridin-2-yl}piperazin-1-yl)ethan-1-one (referred to as Compound 2) on CFA-induced hyperalgesia in the rat at one hour and four hours post dose (left to right—vehicle/vehicle; 1 mg/kg Compound 2/vehicle; 3 mg/kg Compound 2/vehicle; 10 mg/kg Compound 2/vehicle; 10 mg/kg Indomethacin/vehicle).

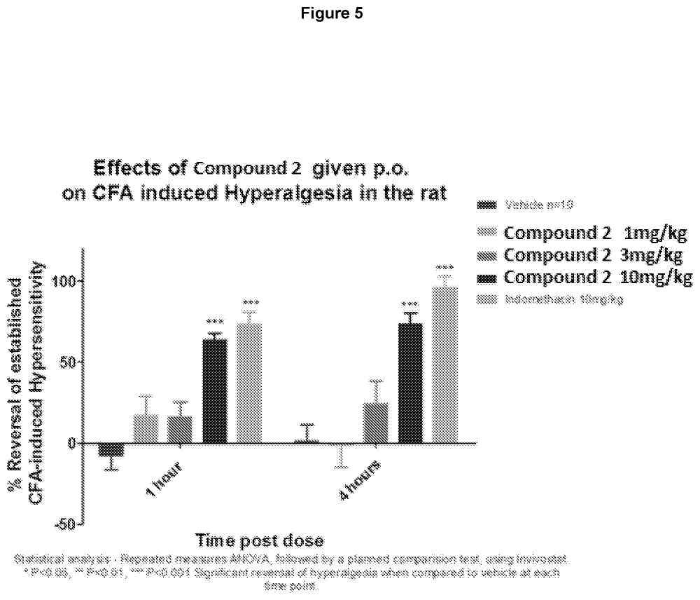

FIG. 6 shows the effect of 1-{5-[3-(4-Fluorophenyl)-3H-imidazo[4,5-c]pyridin-2-yl]pyridin-2-yl}-4-methanesulfonylpiperazine (referred to as Compound 3) on CFA-induced hyperalgesia in the rat at one hour and four hours post dose (left to right—vehicle/vehicle; 1 mg/kg Compound 3/vehicle; 3 mg/kg Compound 3/vehicle; 10 mg/kg Compound 3/vehicle; 10 mg/kg Indomethacin/vehicle).

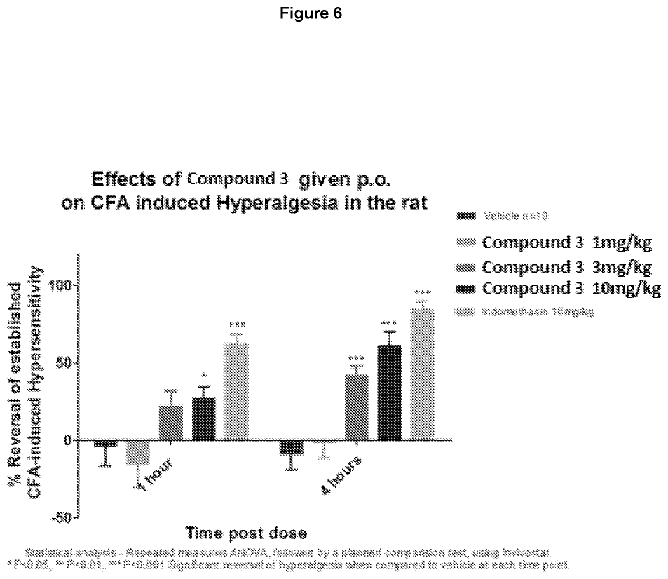

FIG. 7 shows the effect of 4-{5-[3-(4-Fluorophenyl)-3H-imidazo[4,5-c]pyridin-2-yl]pyridin-2-yl}morpholine (referred to as Compound 4) on mechanical allodynia in the rat chronic constriction injury (CCI) model of neuropathic pain (left to right—vehicle/vehicle; 15 mg/kg Compound 4/vehicle; 50 mg/kg Compound 4/vehicle; 150 mg/kg Compound 4/vehicle; 30 mg/kg Pregabalin/vehicle; sham).

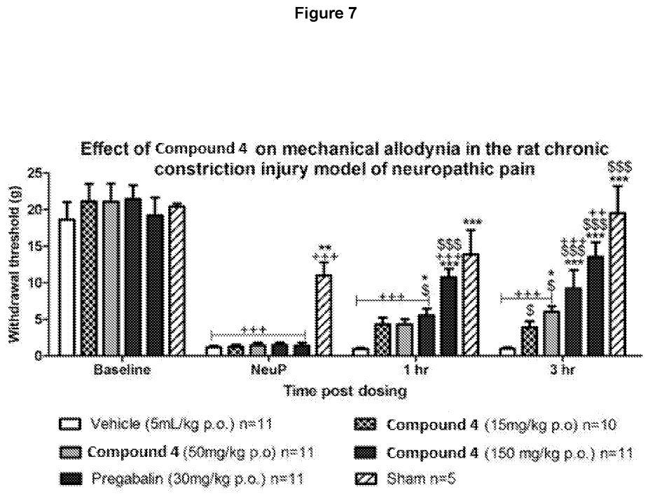

### DETAILED DESCRIPTION OF THE INVENTION

### Definitions

As used herein, the terms “treatment,” “treating,” “treat” and the like, refer to obtaining a desired pharmacologic and/or physiologic effect. In the case of the treatment of migraine, the effect can be prophylactic in terms of completely or partially preventing migraine or a symptom thereof and/or can be therapeutic in terms of a partial or complete cure for migraine and/or an adverse effect attributable to the disease. “Treatment,” as used herein, covers any treatment of migraine in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

As used herein, the term “prevention” refers to the intention to prevent a disease or condition, and includes “prophylactically treating” disease or condition. “Prevention”, as used herein, covers any prevention of migraine in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

An “effective amount” of a VAP-1 inhibitor refers to the amount of a VAP-1 inhibitor that, when administered to a mammal or other subject for preventing or treating a disease or condition, is sufficient to effect such prevention/treatment for the disease or condition. The “effective amount” will vary depending on the VAP-1 inhibitor, the disease and its severity and the age, weight, etc., of the subject to be treated. The therapeutic effect may be objective (i.e., measurable by some test or marker) or subjective (i.e., subject gives an indication of or feels an effect).

The term “VAP-1 inhibitor” or “VAP-1 inhibitor compound” includes both non-biological small molecule inhibitors of VAP-1 and biological inhibitors of VAP-1, including but not limited to RNA, antibodies, polypeptide or proteinaceous inhibitors of VAP-1.

For present purposes, a “VAP-1 inhibitor” or “VAP-1 inhibitor compound” is one which has an IC₅₀ value of less than 1000 nM in the VAP-1 Assay described below.

“Pharmaceutically acceptable” means being useful in preparing a pharmaceutical composition that is generally safe, non-toxic and neither biologically nor otherwise undesirable and includes being useful for veterinary use as well as human pharmaceutical use. Suitable pharmaceutically acceptable salts include, for example acid addition salts derived from inorganic or organic acids, such as hydrochlorides, hydrobromides, p-toluenesulphonates, phosphates, sulphates, perchlorates, acetates, trifluoroacetates, propionates, citrates, malonates, succinates, lactates, oxalates, tartrates and benzoates. For a review on salts, see Handbook of Pharmaceutical Salts: Properties, Selection, and Use by Stahl and Wermuth (Wiley-VCH, Weinheim, Germany, 2002). Pharmaceutically acceptable salts may also be formed with bases. Such salts include salts derived from inorganic or organic bases, for example alkali metal salts such as magnesium or calcium salts, and organic amine salts such as morpholine, piperidine, dimethylamine or diethylamine salts.

The term “migraine” as used herein includes headache, chronic migraine; episodic migraine; medication overuse headache disorder (MOU); migraine without aura; migraine with aura; migraine aura without headache; ocular migraine; vestibular migraine; basilar migraine; hemiplegic migraine; ophthalmoplegic migraine; and tension-type headache (TTH).

Medication overuse headache disorder (MOU) may be described as a self-sustaining, rhythmic, headache medication cycle characterized by daily or near daily headache with irresistible and predictable use of immediate relief medications. Medication overuse headache disorder may also include a chronic headache (occurring on more than 15 days each month) that develops or worsens with frequent use of any drug treatment for pain in people who have tension-type headache (TTH) or migraine.

Chronic migraine may be defined as more than fifteen headache days per month over a three month period of which more than eight are migrainous, in the absence of medication overuse. Episodic migraine is the other migraine sub-type, which is defined as less than 15 headache days per month.

Migraines with aura and migraine without aura are two types of migraine. In migraine without aura suffers do not experience the visual or sensory warning signs sometimes called migraine prodrome or aura.

In migraine aura without headache, a subject may experience aura, nausea, photophobia, hemiparesis, and other migraine symptoms, but does not experience headache. It may sometimes be distinguished from visual-only migraine aura without headache, also called ocular migraine.

Vestibular migraine (also known as migraine associated vertigo or MAV) is typically characterized by unilateral onset of head pain, severe progressive intensity of pain, throbbing or pounding, and interference with a subject's routine activities. Accompanying symptoms of photophobia (sensitivity to light) or phonosensitivity (intolerance to noise), as well as nausea and/or vomiting, are common, and often leads to the inability to perform daily tasks.

Basilar migraines may be headaches that start in the lower part of the brain, i.e. the brainstem. They may cause symptoms such as dizziness, double vision, and lack of coordination. These changes, called an aura, may happen about 10 minutes to 45 minutes before a subject's head hurts.

A subject that suffers hemiplegic migraine may experience a temporary weakness on one side of their body as part of their migraine attack. This may involve the face, arm or leg and be accompanied by numbness, or pins and needles. The person may experience speech difficulties, vision problems or confusion. This may be a frightening experience for the individual as these symptoms are similar to those of a stroke. This weakness may last from one hour to several days, but usually it goes within 24 hours. The head pain associated with migraine typically follows the weakness, but the headache may precede it or be absent.

Typical clinical presentation of ophthalmoplegic migraine generally involves transient migraine-like headache accompanied by often long-lasting oculomotor, abducens or, rarely, trochlear neuropathy with diplopia and (if oculomotor nerve is involved) pupillary abnormalities and ptosis. Ophthalmoplegic migraine generally occurs in children, but a number of adult cases have been reported.

A tension-type headache is a common type of headache and may be considered by some as a “normal” headache. It may feel like a constant ache that affects both sides of the head. The subject may also feel the neck muscles tighten and a feeling of pressure behind the eyes. A tension headache normally won't be severe enough to prevent a subject from their everyday activities. It usually lasts for 30 minutes to several hours, but can last for several days.

### VAP-1 Inhibitors

According to a first aspect of the invention there is provided a compound of formula (I) or a pharmaceutically acceptable salt, or N-oxide thereof:

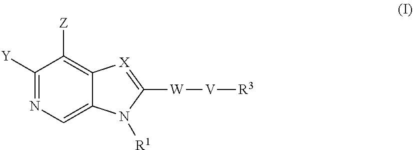

Y is selected from hydrogen, hydroxyl, —NH₂, —NH—C₁₋₄-alkyl, —NH-halo-C₁₋₄-alkyl, or —C₁₋₄-alkoxy;

Z is selected from hydrogen, halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₂, —NHC₁₋₄-alkyl, or —NHhalo-C₁₋₄-alkyl;

R¹ is a phenyl ring, or a 5 or 6-membered heteroaryl ring, either ring being optionally substituted with one or more substituents selected from halogen, cyano, C₁₋₄-alkyl, halo-C₁₋₄-alkyl, cyano-C₁₋₄-alkyl, a 3-7 membered cycloalkyl ring, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, and —NR⁶S(O)₂R⁵; wherein

R^{4A}, R^{4B} R⁵ and R⁶ are each independently selected from hydrogen, C₁₋₄-alkyl or halo-C₁₋₄-alkyl, or

R^{4A} and R^{4B} together with the nitrogen to which they are attached form a 3-7 membered cyclic amino group, optionally substituted by one or more substituents selected from: halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₂, —NHC₁₋₄-alkyl, —NHhalo-C₁₋₄-alkyl;

X is selected from —N═ or —C(R²)═;

R² is selected from hydrogen, halogen, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵;

W is a phenyl ring or a 5 or 6-membered heteroaryl ring, either ring being optionally substituted with one or more substituents selected from halogen, cyano, oxo C₁₋₄-alkyl, halo-C₁₋₄-alkyl, cyano-C₁₋₄-alkyl, —OR⁵, —NR^{7A}R^{7B}, —NR⁶C(O)OR⁵, —NR⁶C(O)R⁵, —NR⁶C(O)NR^{7A}R^{7B}, —C(O)NR^{7A}R^{7B}, —C(O)R⁵, —C(O)OR⁵, —SO₂R⁵, —SO₂NR^{7A}R^{7B} and —NR⁶S(O)₂R⁵;

R^{7A} and R^{7B} are independently hydrogen, C₁₋₄-alkyl or halo-C₁₋₄-alkyl.

V is selected from a bond, —O—, —N(R⁶)—, —(C═O)—, —CONR⁶—, —NR⁶C(O)—, or —C₁₋₄-alkylene-, wherein the C₁₋₄-alkylene group is optionally substituted by halogen, and wherein any one of the carbon atoms of the C₁₋₄-alkylene group may be replaced by —O— or —N(R⁶)—;

R³ is selected from hydrogen, —C₁₋₄-alkyl, —C₁₋₄-alkyl-C₁₋₄-alkoxy or a 3-7 membered heterocyclic ring or 3-7 membered cycloalkyl ring, or a 5 or 6-membered heteroaryl ring, any one of the rings being optionally substituted with one or more substituents selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵;

with the proviso that groups —W—V—R³ and/or R¹ are not

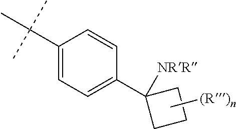

wherein

n is 0, 1, or 2;

R′ and R″ are independently selected from the group consisting of H, —C₁-C₆alkyl, —(C═O)—C₁-C₆ alkyl and —(C═O)OC(CH₃)₃; and

R′″ is H, OH, or C₁-C₆ alkyl.

According to the second aspect of the invention there is provided a compound of formula (I) or a pharmaceutically acceptable salt, or N-oxide thereof

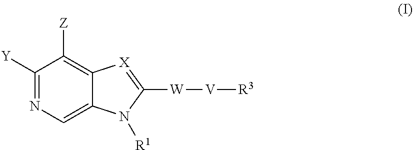

wherein:

Y is selected from hydrogen, hydroxyl, —NH₂, —NH—C₁₋₄-alkyl, —NH-halo-C₁₋₄-alkyl, or —C₁₋₄-alkoxy;

Z is selected from hydrogen, halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₂, —NHC₁₋₄-alkyl, or —NHhalo-C₁₋₄-alkyl;

R¹ is a phenyl ring, or a 5 or 6-membered heteroaryl ring, either ring being optionally substituted with one or more substituents selected from halogen, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, and —NR⁶S(O)₂R⁵; wherein

R^{4A}, R^{4B} R⁵ and R⁶ are each independently selected from hydrogen, C₁₋₄-alkyl or halo-C₁₋₄-alkyl, or

R^{4A} and R^{4B} together with the nitrogen to which they are attached form a 3 to 7-membered cyclic amino group, optionally substituted by one or more substituents selected from: halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₂, —NHC₁₋₄-alkyl, —NHhalo-C₁₋₄-alkyl;

X is selected from —N═ or —C(R²)═;

R² is selected from hydrogen, halogen, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵;

W is a phenyl ring or a 5 or 6-membered heteroaryl ring, either ring being optionally substituted with one or more substituents selected from halogen, cyano, C₁₋₄-alkyl, halo-C₁₋₄-alkyl, cyano-C₁₋₄-alkyl, —OR⁵, —NR^{7A}R^{7B}, —NR⁶C(O)OR⁵, —NR⁶C(O)R⁵, —NR⁶C(O)NR^{7A}R^{7B}, —C(O)NR^{7A}R^{7B}, —C(O)R⁵, —C(O)OR⁵, —SO₂R⁵, —SO₂NR^{7A}R^{7B} and —NR⁶S(O)₂R⁵;

R^{7A} and R^{7B} are independently hydrogen, C₁₋₄-alkyl or halo-C₁₋₄-alkyl.

V is selected from a bond, —O—, —N(R⁶)—, —(C═O)—, —CONR⁶—, —NR⁶C(O)—, or —C₁₋₄-alkylene-, wherein the C₁₋₄-alkylene group is optionally substituted by halogen, and wherein any one of the carbon atoms of the C₁₋₄-alkylene group may be replaced by —O— or —N(R⁶)—; R³ is hydrogen or a 3-7 membered heterocyclic ring or 3-7 membered cycloalkyl ring selected from cyclopropyl, cyclopentyl or cyclohexyl, or a 5 or 6-membered heteroaryl ring, any one of the rings being optionally substituted with one or more substituents selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵.

In a feature of the first and/or second aspect of the invention, in the VAP-1 inhibitor

(i) Y may be hydrogen;

(ii) Z may be hydrogen;

(iii) R¹ may be phenyl or 6-membered heteroaryl, optionally substituted with one or more substituents selected from halogen, C₁₋₄-alkyl or halo-C₁₋₄-alkyl; preferably R¹ may be phenyl or pyridyl, optionally substituted with one or more substituents selected from F, Cl or CH₃; and/or

(iv) X is —C(R²)═, and R² may be hydrogen, halogen, cyano, C₁₋₄-alkyl, or halo-C₁₋₄-alkyl; preferably R² may be hydrogen.

In a further feature of the first and/or second aspect of the invention, in the VAP-1 inhibitors, W may be

(a) a phenyl ring optionally substituted with one or more substituents as defined above;

(b) a 6-membered heteroaryl ring selected from pyridine, pyridazine, pyrazine, or pyrimidine optionally substituted with one or more substituents as defined above;

(c) a 5-membered heteroaryl ring selected from oxazole, thiazole or imidazole optionally substituted with one or more substituents as defined above; or

(d) an imidazolyl ring optionally substituted as above, and wherein the imidazolyl ring is connected to the pyrrolopyridine core via an imidazolyl ring carbon atom.

In a further feature, W may be optionally substituted with one or more substituents selected from fluoro, chloro, cyano, CH₃ or CF₃.

In another feature,

(A) V may be —CH₂—, —(CH₂)₂—, or —N(R⁶)CH₂—, or —CH₂—N(R⁶)—, optionally wherein, when dependent on claim 12, R³ is cyclopropyl, cyclobutyl, cyclopentyl or cyclohexyl optionally substituted as defined above;

(B) R³ may be formed from —NR^{4A}R^{4B} wherein R^{4A} and R^{4B}, together with the nitrogen atom to which they are attached join together to form a 4 to 7-membered heterocyclic ring optionally substituted as defined above; or

(C) R³ is selected from the group consisting of:

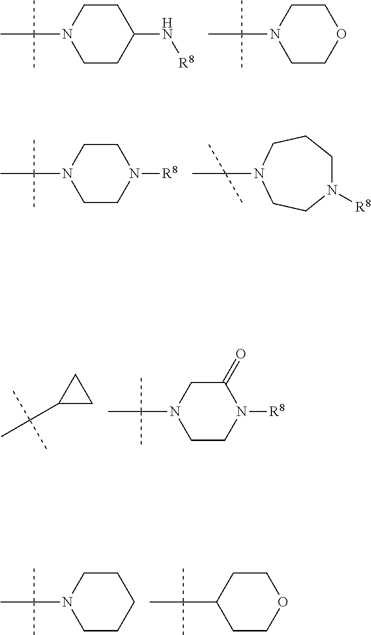

wherein R⁸ may be selected from hydrogen, CH₃, —CONH₂, —NHCONH₂, —S(O)₂CH₃, —COCH₃

Specific Examples of VAP-1 inhibitors of the first and second aspect of the innovation include those disclosed in WO2014/140592, which is incorporated herein by reference. Those VAP-1 inhibitors include those set out in the following table

| | | | Intermediate(s), Form, |
|---|---|---|---|
| Ex | Structure | Name | Yield, LCMS, HPLC |
| 1 | 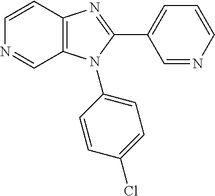 [SMILES: Clc1ccc(-n2c(-c3cccnc3)nc3ccncc32)cc1] | 3-[3-(4-Chlorophenyl)-3H- imidazo[4,5-c]pyridin-2- yl]pyridine | White solid 139 mg, 49.5% HRMS (ESI⁺) calcd for [MH]⁺ of C₁₇H₁₁ClN₄ 307.0750 found 307.0748. HPLC: Rt 4.22 min, 99.8% purity. |
| 2 | 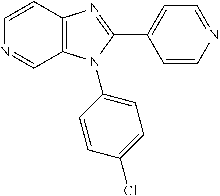 [SMILES: Clc1ccc(-n2c(-c3ccncc3)nc3ccncc32)cc1] | 4-[3-(4-Chlorophenyl)-3H- imidazo[4,5-c]pyridin-2- yl]pyridine | Light pink solid Yield 109 mg, 52.7% HRMS (ESI⁺) calcd for [MH]⁺ of C₁₇H₁₁ClN₄ 307.0750 found 307.0752. HPLC: Rt 3.90 min, 99.8% purity |
| 3 | 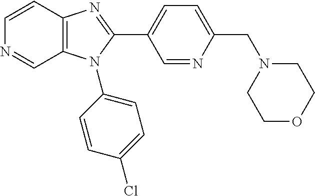 [SMILES: Clc1ccc(-n2c(-c3ccc(CN4CCOCC4)nc3)nc3ccncc32)cc1] | 4-({5-[3-(4-Chlorophenyl)- 3H-imidazo[4,5-c]pyridin- 2-yl]pyridin-2- yl}methyl)morpholine | White solid Yield 105 mg, 38.6% HRMS (ESI⁺) calcd for [MH]⁺of C₂₂H₂₀ClN₅O 406.1435 found 406.1428. HPLC: Rt 3.65 min, 100% purity |
| 4 | 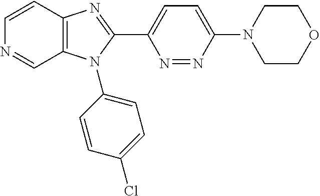 [SMILES: Clc1ccc(-n2c(-c3ccc(N4CCOCC4)nn3)nc3ccncc32)cc1] | 4-{6-[3-(4-Chlorophenyl)- 3H-imidazo[4,5-c]pyridin- 2-yl]pyridazin-3- yl}morpholine | White solid Yieldd 89.9 mg, 18.8% HRMS (ESI⁺) calcd for [MH]⁺ of C₂₀H₁₇ClN₅O 393.1230 found 393.1234. HPLC: Rt 4.71 min, 98.4% purity |
| 5 | 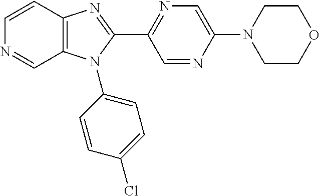 [SMILES: Clc1ccc(-n2c(-c3cnc(N4CCOCC4)cn3)nc3ccncc32)cc1] | 4-{5-[3-(4-Chlorophenyl)- 3H-imidazo[4,5-c]pyridin- 2-yl]pyrazin-2- yl}morpholine | While solid Yield 114 mg, 18.9% HRMS (ESI⁺) calcd for [MH]⁺ of C₂₀H₁₇ClN₆O 393.1230 found 393.1234. HPLC: Rt 4.79 min, 100% purity |
| 6 | 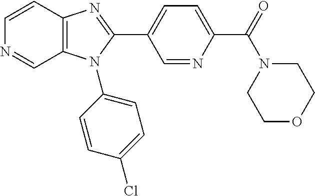 [SMILES: O=C(c1ccc(-c2nc3ccncc3n2-c2ccc(Cl)cc2)cn1)N1CCOCC1] | 4-({5-[3-(4-Chlorophenyl)- 3H-imidazo[4,5-c]pyridin- 2-yl]pyridin-2- yl}carbonyl)morpholine | White solid Yield 170 mg, 40.5% HRMS (ESI⁺) calcd for [MH]⁺ of C₂₂H₁₈ClN₅O₂ 420.1227 found 420.1228. HPLC: Rt 4.03 min, 100% purity |
| 7 | 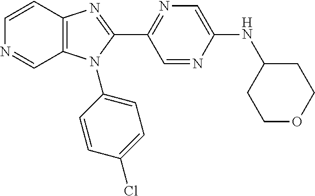 [SMILES: Clc1ccc(-n2c(-c3cnc(NC4CCOCC4)cn3)nc3ccncc32)cc1] | 5-[3-(4-Chlorophenyl)-3H- imidazo[4,5-c]pyridin-2- yl]-N-(oxan-4-yl)pyrazin-2- amine | Yellow solid Yield 50.0 mg, 30.0% HRMS (ESI⁺) calcd for [MH]⁺ of C₂₁H₁₉ClN₆O 407.1387 found 407.1380. HPLC: Rt 4.86 min, 100% purity |
| 8 | 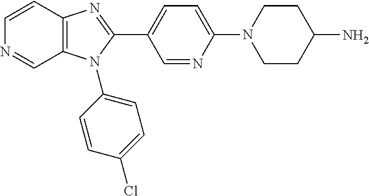 [SMILES: NC1CCN(c2ccc(-c3nc4ccncc4n3-c3ccc(Cl)cc3)cn2)CC1] | 1-{5-[3-(4-Chlorophenyl)- 3H-imidazo[4,5-c]pyridin- 2-yl]pyridin-2-yl}piperidin- 4-amine | White solid Yield 24.2 mg, 38.7% HRMS (ESI⁺) calcd for [MH]⁺ of C₂₂H₂₁ClN₆ 405.1594 found 405.1591. HPLC: Rt 3.52 min, 100% purity |
| 9 | 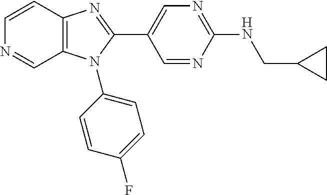 [SMILES: Fc1ccc(-n2c(-c3cnc(NCC4CC4)nc3)nc3ccncc32)cc1] | N-(Cyclopropylmethyl)-5- [3-(4-fluorophenyl)-3H- imidazo[4,5-c]pyridin-2- yl]pyrimidin-2-amine | White solid Yield 24.6 mg, 8.28% HRMS (ESI⁺) calcd for [MH]⁺ of C₂₀H₁₇FN₆ 361.1577 found 361.1594. HPLC: Rt 5.01 min, 97.0% purity |
| 10 | 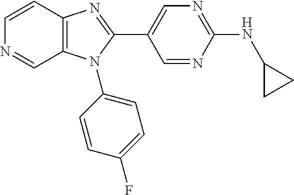 [SMILES: Fc1ccc(-n2c(-c3cnc(NC4CC4)nc3)nc3ccncc32)cc1] | N-Cyclopropyl-5-[3-(4- fluorophenyl)-3H- imidazo[4,5-c]pyridin-2- yl]pyrimidin-2-amine | White solid Yield 30.9 mg, 4.93% HRMS (ESI⁺) calcd for [MH]⁺ of C₁₉H₁₅FN₆ 347.1420 found 347.1422. HPLC: Rt 4.46 min, 99.6% purity |
| 11 | 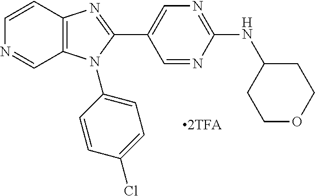 [SMILES: Clc1ccc(-n2c(-c3cnc(NC4CCOCC4)nc3)nc3ccncc32)cc1 ; "·2TFA"] | 5-[3-(4-Chlorophenyl)-3H- imidazo[4,5-c]pyridin-2- yl]-N-(oxan-4-yl)pyrimidin- 2-amine; bis(trifluoroacetic acid) | White solid Yield 73.8 mg, 5.39% HRMS (ESI⁺) calcd for [MH]⁺ of C₂₁H₁₉ClN₆O 407.1387 found 407.1403. HPLC: Rt 4.76 min, 100% purity |
| 12 | 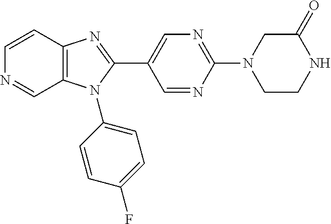 [SMILES: O=C1CN(c2ncc(-c3nc4ccncc4n3-c3ccc(F)cc3)cn2)CCN1] | 4-{5-[3-(4-Fluorophenyl)- 3H-imidazo[4,5-c]pyridin- 2-yl]pyrimidin-2- yl}piperazin-2-one | White solid Yield 104 mg, 9.29% HRMS (ESI⁺) calcd for [MH]⁺ of C₂₀H₁₆FN₇O 390.1479 found 390.1481. HPLC: Rt 4.01 min, 100% purity |
| 13 | 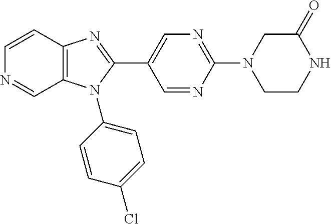 [SMILES: O=C1CN(c2ncc(-c3nc4ccncc4n3-c3ccc(Cl)cc3)cn2)CCN1] | 4-{5-[3-(4-Chlorophenyl)- 3H-imidazo[4,5-c]pyridin- 2-yl]pyrimidin-2- yl}piperazin-2-one | White solid Yield 97.1 mg, 14.2% HRMS (ESI⁺) calcd for [MH]⁺ of C₂₀H₁₆ClN₇O 406.1183 found 406.1185. HPLC: Rt 4.25 min, 99.5% purity |
| 14 | 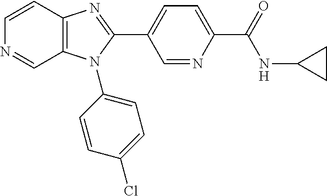 [SMILES: O=C(NC1CC1)c1ccc(-c2nc3ccncc3n2-c2ccc(Cl)cc2)cn1] | 5-[3-(4-Chlorophenyl)-3H- imidazo[4,5-c]pyridin-2- yl]-N-cyclopropylpyridine- 2-carboxamide | White solid Yield 100 mg, 13.3% HRMS (ESI⁺) calcd for [MH]⁺ of C₂₁H₁₆ClN₅O 390.1122 found 390.1139. HPLC: Rt 4.81 min, 100% purity |
| 15 | 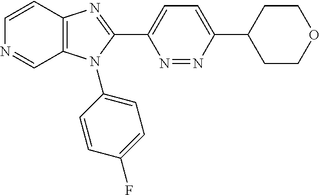 [SMILES: Fc1ccc(-n2c(-c3ccc(C4CCOCC4)nn3)nc3ccncc32)cc1] | 3-[3-(4-Fluorophenyl)-3H- imidazo[4,5-c]pyridin-2- yl]-6-(oxan-4-yl)pyridazine | Off white solid Yield 14.0 mg, 6.63% HRMS (ESI⁺) calcd for [MH]⁺ of C₂₁H₁₈FN₅O 376.1573 found 376.1575. HPLC: Rt 4.37 min, 98.4% purity |
| 16 | 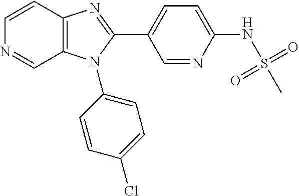 [SMILES: CS(=O)(=O)Nc1ccc(-c2nc3ccncc3n2-c2ccc(Cl)cc2)cn1] | N-{5-[3-(4-Chlorophenyl)- 3H-imidazo[4,5-c]pyridin- 2-yl]pyridin-2- yl}methanesulfonamide | White solid Yield 163 mg, 48.8% HRMS (ESI⁺) calcd for [MH]⁺ of C₁₈H₁₄ClN₅O₂S 400.0635 found 400.0631. HPLC: Rt 4.19 min, 100% purity |
| 17 | 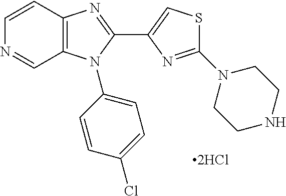 [SMILES: Clc1ccc(-n2c(-c3csc(N4CCNCC4)n3)nc3ccncc32)cc1 ; "·2HCl"] | 1-{4-[3-(4-Chlorophenyl)- 3H-imidazo[4,5-c]pyridin- 2-yl]-1,3-thiazol-2- yl}piperazine dihydrochloride | Yellow solid Yield 47.3 mg, 17.3% * HRMS (ESI⁺) calcd for [MH]⁺ of C₁₉H₁₇ClN₆S 397.1002 found 397.1011. HPLC: Rt 3.44-3.55 min, 100% purity |
| 18 | 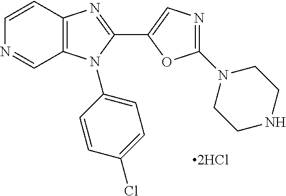 [SMILES: Clc1ccc(-n2c(-c3cnc(N4CCNCC4)o3)nc3ccncc32)cc1 ; "·2HCl"] | 1-{5-[3-(4-Chlorophenyl)- 3H-imidazo[4,5-c]pyridin- 2-yl]-1,3-oxazol-2- yl}piperazine dihydrochloride | Orange solid Yield 58.0 mg, 11.8% * HRMS (ESI⁺) calcd for [MH]⁺ of C₁₉H₁₇ClN₆O 381.1230 found 381.1241. HPLC: Rt 3.25 min, 100% purity |
| 19 | 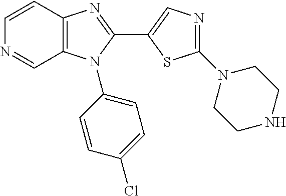 [SMILES: Clc1ccc(-n2c(-c3cnc(N4CCNCC4)s3)nc3ccncc32)cc1] | 1-{5-[3-(4-Chlorophenyl)- 3H-imidazo[4,5-c]pyridin- 2-yl]-1,3-thiazol-2- yl}piperazine | Off white solid Yield 9.50 mg, 2.96% * HRMS (ESI⁺) calcd for [MH]⁺ of C₁₉H₁₇ClN₆S 397.1002 found 397.1008. HPLC: Rt 3.41-3.53 min, 99.3% purity |
| 20 | 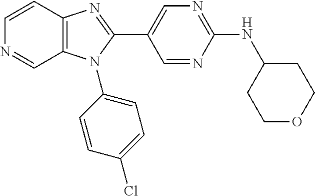 [SMILES: Clc1ccc(-n2c(-c3cnc(NC4CCOCC4)nc3)nc3ccncc32)cc1] | 5-[3-(4-Fluorophenyl)-3H- imidazo[4,5-c]pyridin-2- yl]-N-(oxan-4-yl)pyrimidin- 2-amine | White solid Yield 79.0 mg, 13.9% HRMS (ESI⁺) calcd for [MH]⁺ of C₂₁H₁₉FN₆O 391.1682 found 391.1693. HPLC: Rt 4.47 min, 100% purity |
| 21 | 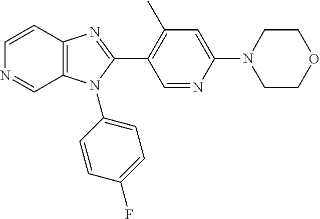 [SMILES: Cc1cc(N2CCOCC2)ncc1-c1nc2ccncc2n1-c1ccc(F)cc1] | 4-{5-[3-(4-Fluorophenyl)- 3H-imidazo[4,5-c]pyridin- 2-yl]-4-methylpyridin-2- yl}morpholine | White solid Yield 15.2 mg, 2.17% HRMS (ESI⁺) calcd for [MH]⁺ of C₂₂H₂₀FN₅O 390.1730 found 390.1721. HPLC: Rt 4.09 min, 98.2% purity |
| 22 | 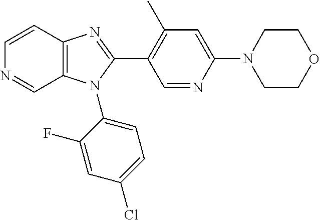 [SMILES: Cc1cc(N2CCOCC2)ncc1-c1nc2ccncc2n1-c1ccc(Cl)cc1F] | 4-{5-[3-(4-Chloro-2- fluorophenyl)-3H- imidazo[4,5-c]pyridin-2- yl]-4-methylpyridin-2- yl}morpholine | Yellow solid Yield 16.0 mg, 3.59% HRMS (ESI⁺) calcd for [MH]⁺ of C₂₂H₁₉ClFN₅O 424.1340 found 424.134. HPLC: Rt 4.31 min, 98.4% purity |
| 23 | 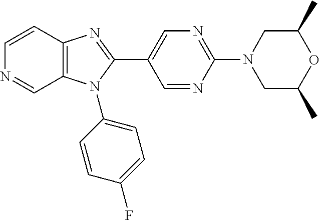 [SMILES: C[C@@H]1CN(c2ncc(-c3nc4ccncc4n3-c3ccc(F)cc3)cn2)C[C@H](C)O1] | (2R,6S)-4-{5-[3-(4- Fluorophenyl)-3H- imidazo[4,5-c]pyridin-2- yl]pyrimidin-2-yl}-2,6- dimethylmorpholine | Beige solid Yield 25.8 mg, 6.39% HRMS (ESI⁺) calcd for [MH]⁺ of C₂₂H₂₁FN₆O 405.1839 found 405.1843. HPLC: Rt 5.14 min, 99.1% purity |
| 24 | 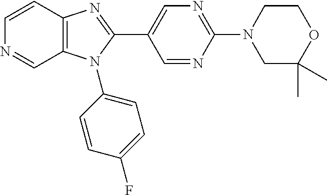 [SMILES: CC1(C)CN(c2ncc(-c3nc4ccncc4n3-c3ccc(F)cc3)cn2)CCO1] | 4-{5-[3-(4-Fluorophenyl)- 3H-imidazo[4,5-c]pyridin- 2-yl]pyrimidin-2-yl}-2,2- dimethylmorpholine | White solid Yield 17.1 mg, 5.28% HRMS (ESI⁺) calcd for [MH]⁺ of C₂₂H₂₁FN₆O 405.1839 found 405.1852. HPLC: Rt 5.14 min, 97.4% purity |
| 25 | 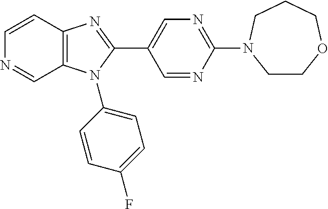 [SMILES: Fc1ccc(-n2c(-c3cnc(N4CCCOCC4)nc3)nc3ccncc32)cc1] | 4-{5-[3-(4-Fluorophenyl)- 3H-imidazo[4,5-c]pyridin- 2-yl]pyrimidin-2-yl}-1,4- oxazepane | White solid Yield 415 mg, 43.5% HRMS (ESI⁺) calcd for [MH]⁺ of C₂₁H₁₉FN₆O 391.1682 found 391.1682. HPLC: Rt 4.80 min, 98.7% purity |
| 26 | 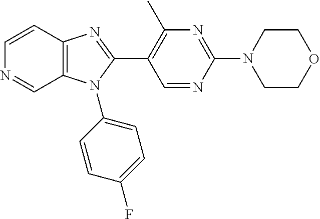 [SMILES: Cc1nc(N2CCOCC2)ncc1-c1nc2ccncc2n1-c1ccc(F)cc1] | 4-{5-[3-(4-Fluorophenyl)- 3H-imidazo[4,5-c]pyridin- 2-yl]-4-methylpyrimidin-2- yl}morpholine | White solid Yield 8.50 mg, 1.76% HRMS (ESI⁺) calcd for [MH]⁺ of C₂₁H₁₉FN₆O 391.1682 found 391.1676. HPLC: Rt 4.54 min, 97.8% purity |
| 27 | 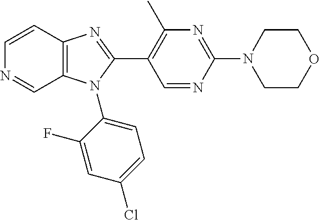 [SMILES: Cc1nc(N2CCOCC2)ncc1-c1nc2ccncc2n1-c1ccc(Cl)cc1F] | 4-{5-[3-(4-Chloro-2- fluorophenyl)-3H- imidazo[4,5-c]pyridin-2- yl]-4-methylpyrimidin-2- yl}morpholine | White solid Yield 37.0 mg, 8.28% HRMS (ESI⁺) calcd for [MH]⁺ of C₂₁H₁₈ClFN₆O 425.1293 found 425.1296. HPLC: Rt 4.92 min, 99.8% purity |
| 28 | 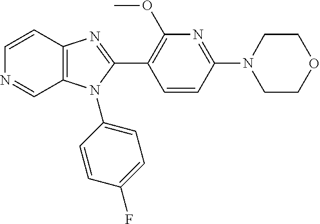 [SMILES: COc1nc(N2CCOCC2)ccc1-c1nc2ccncc2n1-c1ccc(F)cc1] | 4-{5-[3-(4-Fluorophenyl)- 3H-imidao[4,5-c]pyridin- 2-yl]-6-methoxypyridin-2- yl}morpholine | Pale yellow solid Yield 47.4 mg, 41.6% HRMS (ESI⁺) calcd for [MH]⁺ of C₂₂H₂₀FN₅O₂ 406.1679 found 406.1683. HPLC: Rt 5.02 min, 99.5% purity |
| 29 | 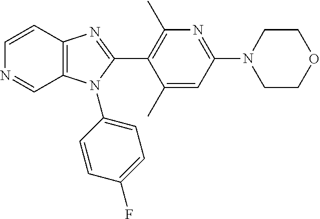 [SMILES: Cc1cc(N2CCOCC2)nc(C)c1-c1nc2ccncc2n1-c1ccc(F)cc1] | 4-{5-[3-(4-Fluorophenyl)- 3H-imidazo[4,5-c]pyridin- 2-yl]-4,5-dimethylpyridin- 2-yl}morpholine | Off white solid Yield 15.0 mg, 2.00% HRMS (ESI⁺) calcd for [MH]⁺ of C₂₃H₂₂FN₅O 404.1887 found 404.1889. HPLC: Rt 3.74 min, 99.5% purity |
| 30 | 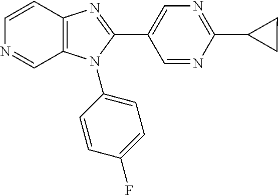 [SMILES: Fc1ccc(-n2c(-c3cnc(C4CC4)nc3)nc3ccncc32)cc1] | 2-Cyclopropyl-5-[3-(4- fluorophenyl)-3H- imidazo[4,5-c]pyridin-2- yl]pyrimidine | Off white solid Yield 50.2 mg, 17.3% HRMS (ESI⁺) calcd for [MH]⁺ of C₁₉H₁₄FN₅ 332.1311 found 332.1313. HPLC: Rt 4.59 min, 99.7% purity |
| 31 | 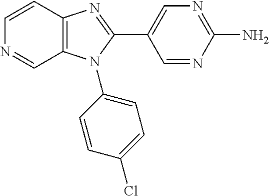 [SMILES: Nc1ncc(-c2nc3ccncc3n2-c2ccc(Cl)cc2)cn1] | 5-[3-(4-Chlorophenyl)-3H- imidazo[4,5-c]pyridin-2- yl]pyrimidin-2-amine | Off white solid Yield 14.0 mg, 3.16% HRMS (ESI⁺) calcd for [MH]⁺ of C₁₆H₁₁ClN₆ 323.0812 found 323.0815. HPLC: Rt 3.79 min, 100% purity |
| 32 | 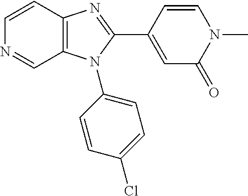 [SMILES: Cn1ccc(-c2nc3ccncc3n2-c2ccc(Cl)cc2)cc1=O] | 4-[3-(4-Chlorophenyl)-3H- imidazo[4,5-c]pyridin-2- yl]-1-methyl-1,2- dihydropyridin-2-one | White solid Yield 109 mg, 17.7% HRMS (ESI⁺) calcd for [MH]⁺ of C₁₈H₁₃ClN₄O 337.0856 found 337.0859. HPLC: Rt 4.07 min, 98.8% purity |
| 33 | 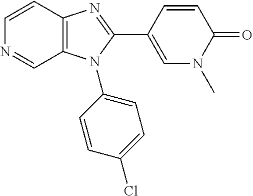 [SMILES: Cn1cc(-c2nc3ccncc3n2-c2ccc(Cl)cc2)ccc1=O] | 5-[3-(4-Chlorophenyl)-3H- imidazo[4,5-c]pyridin-2- yl]-1-methyl-1,2- dihydropyridin-2-one | Off white solid Yield 24.0 mg, 7.83% HRMS (ESI⁺) calcd for [MH]⁺ of C₁₈H₁₃ClN₄O 337.0856 found 337.0857. HPLC: Rt 4.14 min, 99.6% purity |
| 34 | 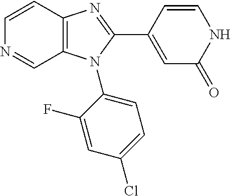 [SMILES: O=c1cc(-c2nc3ccncc3n2-c2ccc(Cl)cc2F)cc[nH]1] | 4-[3-(4-Chloro-2- fluorophenyl)-3H- imidazo[4,5-c]pyridin-2- yl]-1,2-dihydropyridin-2- one | White solid Yield 15.0 mg, 1.05% HRMS (ESI⁺) calcd for [MH]⁺ of C₁₇H₁₀ClFN₄O 341.0605 found 341.0607. HPLC: Rt 3.55 min, 100% purity |
| 35 | 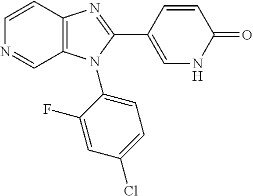 [SMILES: O=c1ccc(-c2nc3ccncc3n2-c2ccc(Cl)cc2F)c[nH]1] | 5-[3-(4-Chloro-2- fluorophenyl)-3H- imidazo[4,5-c]pyridin-2- yl]-1,2-dihydropyridin-2- one | White solid Yield 41.0 mg, 55.72% HRMS (ESI⁺) calcd for [MH]⁺ of C₁₇H₁₀ClFN₄O 341.0605 found 341.0613. HPLC: Rt 3.65 min, 100% purity |
| 36 | 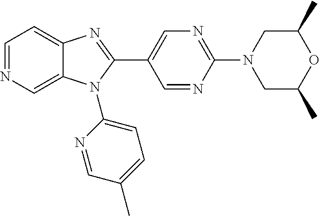 [SMILES: Cc1ccc(-n2c(-c3cnc(N4C[C@@H](C)O[C@@H](C)C4)nc3)nc3ccncc32)nc1] | (2R,6S)-2,6-Dimethyl-4- {5-[3-(5-methylpyridin-2- yl)-3H-imidazo[4,5- c]pyridin-2-yl)pyrimidin-2- yl}morpholine | White solid Yield 26.0 mg, 3.71% HRMS (ESI⁺) calcd for [MH]⁺ of C₂₂H₂₃N₇O 402.2042 found 402.2047. HPLC: Rt 4.96 min, 98.8% purity |
| 37 | 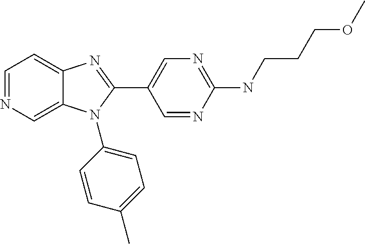 [SMILES: COCCCNc1ncc(-c2nc3ccncc3n2-c2ccc(C)cc2)cn1] | N-(3-Methoxypropyl)-5-[3- (4-methylphenyl)-3H- imidazo[4,5-c]pyridin-2- yl]pyrimidin-2-amine | White solid Yield 65.0 mg, 8.65% HRMS (ESI⁺) calcd for [MH]⁺ of C₂₁H₂₂N₆O 375.1933 found 375.1935. HPLC: Rt 4.66 min, 99.3% purity |
| 38 | 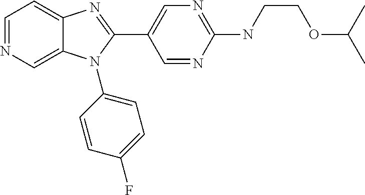 [SMILES: CC(C)OCCNc1ncc(-c2nc3ccncc3n2-c2ccc(F)cc2)cn1] | 5-[3-(4-Fluorophenyl)-3H- imidazo[4,5-c]pyridin-2- yl]-N-[2-(propan-2- yloxy)ethyl]pyrimidin-2- amine | White solid Yield 181 mg, 23.4% HRMS (ESI⁺) calcd for [MH]⁺ of C₂₁H₂₁FN₆O 393.1839 found 393.1823. HPLC: Rt 4.80 min, 99.4% purity |
| 39 | 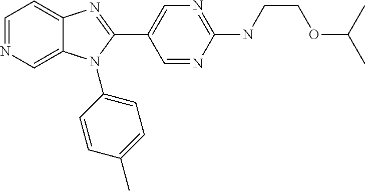 [SMILES: Cc1ccc(-n2c(-c3cnc(NCCOC(C)C)nc3)nc3ccncc32)cc1] | 5-[3-(4-Methylphenyl)-3H- imidazo[4,5-c]pyridin-2- yl]-N-[2-(propan-2- yloxy)ethyl]pyrimidin-2- amine | White solid Yield 55.7 mg, 7.14% HRMS (ESI⁺) calcd for [MH]⁺ of C₂₂H₂₄N₆O 389.2090 found 389.2083. HPLC: Rt 5.01 min, 100% purity |
| 40 | 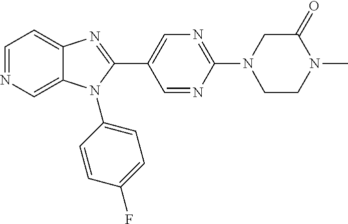 [SMILES: CN1CCN(c2ncc(-c3nc4ccncc4n3-c3ccc(F)cc3)cn2)CC1=O] | 4-{5-[3-(4-Fluorophenyl)- 3H-imidazo[4,5-c]pyridin- 2-yl]pyrimidin-2-yl}-1- methylpiperazin-2-one | White solid Yield 128 mg, 21.5% HRMS (ESI⁺) calcd for [MH]⁺ of C₂₁H₁₈FN₇O 404.1635 found 404.1620. HPLC: Rt 4.22 min, 100% purity |
| 41 | 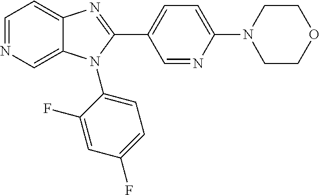 [SMILES: Fc1ccc(-n2c(-c3ccc(N4CCOCC4)nc3)nc3ccncc32)c(F)c1] | 4-{5-[3-(2,4- Difluorophenyl)-3H- imidazo[4,5-c]pyridin-2- yl]pyridin-2-yl}morpholine | White solid Yield 41.1 mg, 6.60% HRMS (ESI⁺) calcd for [MH]⁺ of C₂₁H₁₇F₂N₅O 394.1479 found 394.1469. HPLC: Rt 4.47 min, 99.2% purity |
| 42 | 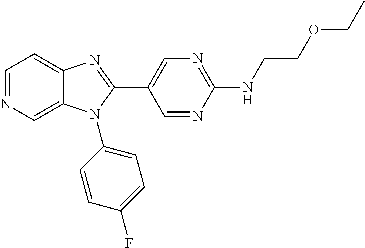 [SMILES: CCOCCNc1ncc(-c2nc3ccncc3n2-c2ccc(F)cc2)cn1] | N-(2-Ethoxyethyl)-5-[3-(4- fluorophenyl)-3H- imidazo[4,5-c]pyridin-2- yl]pyrimidin-2-amine | Off white solid Yield 154 mg, 27.6% LCMS (ES⁺): 379.0 [MH]⁺ HPLC: Rt 4.70 min, 99.1% purity |
| 43 | 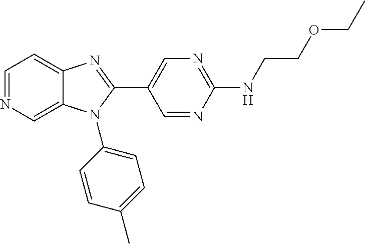 [SMILES: CCOCCNc1ncc(-c2nc3ccncc3n2-c2ccc(C)cc2)cn1] | N-(2-Ethoxyethyl)-5-[3-(4- methylphenyl)-3H- imidazo[4,5-c]pyridin-2- yl]pyrimidin-2-amine | White solid Yield 119 mg, 21.1% LCMS (ES⁺): 375.1 [MH]⁺ HPLC: Rt 4.94 min, 99.1% purity |
| 44 | 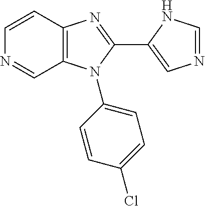 [SMILES: Clc1ccc(-n2c(-c3cnc[nH]3)nc3ccncc32)cc1] | 5-[3-(4-Chlorophenyl)-3H- imidazo[4,5-c]pyridin-2- yl]-1H-imidazole | Yellow solid 51.2 mg, 23.0% HRMS (ESI⁺) calcd for [MH]⁺ of C₁₅H₁₀ClN₅ 296.0703 found 296.0709. HPLC: Rt 3.24 min, 100% purity |
| 45 | 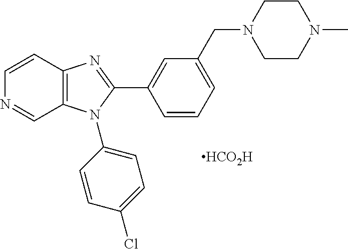 [SMILES: CN1CCN(Cc2cccc(-c3nc4ccncc4n3-c3ccc(Cl)cc3)c2)CC1 ; "·HCO2H"] | 1-({3-[3-(4-Chlorophenyl)- 3H-imidazo[4,5-c]pyridin- 2-yl]phenyl}methyl)-4- methylpiperazine; formic acid | White solid Yield 34.6 mg, 9.91% HRMS (ESI+) calcd for [MH]+ of C24H24ClN5 418.1798 found 418.1794. HPLC: Rt 3.51 min, 99.0% purity |
| 46 | 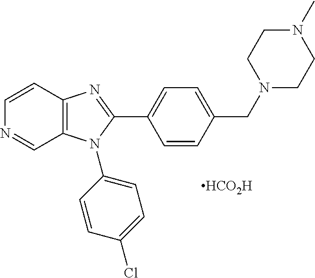 [SMILES: CN1CCN(Cc2ccc(-c3nc4ccncc4n3-c3ccc(Cl)cc3)cc2)CC1 ; "·HCO2H"] | 1-({4-[3-(4-Chlorophenyl)- 3H-imidazo[4,5-c]pyridin- 2-yl]phenyl}methyl)-4- methylpiperazine; formic acid | Light yellow solid Yield 42.0 mg, 12.0% HRMS (ESI+) cacd for [MH]+ of C24H24ClN5 418.1798 found 418.1813. HPLC: Rt 3.43 min, 99.1% purity |
| 47 | 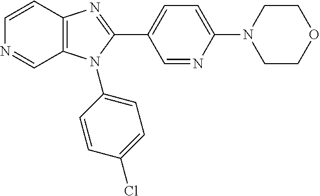 [SMILES: Clc1ccc(-n2c(-c3ccc(N4CCOCC4)nc3)nc3ccncc32)cc1] | 4-{5-[3-(4-Chlorophenyl)- 3H-imidazo[4,5-c]pyridin- 2-yl]pyridin-2- yl}morpholine | White solid Yield 70.9 mg, 20.9% HRMS (ESI+) calcd for [MH]+ of C21H18ClN5O 392.1278 found 392.1282. HPLC: Rt 4.49 min, 100% purity |
| 48 | 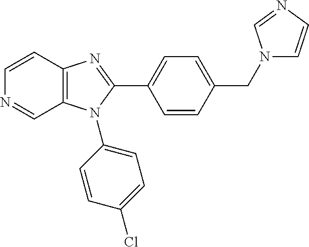 [SMILES: Clc1ccc(-n2c(-c3ccc(Cn4ccnc4)cc3)nc3ccncc32)cc1] | 1-({4-[3-(4-Chlorophenyl)- 3H-imidazo[4,5-c]pyridin- 2-yl]phenyl}methyl)-1H- imidazole | Orange gum Yield 39.4 mg, 11.4% HRMS (ESI+) clacd for [MH]+ of C22H16ClN5 386.1172 found 386.1174. HPLC: Rt 3.90 min, 100% purity |
| 49 | 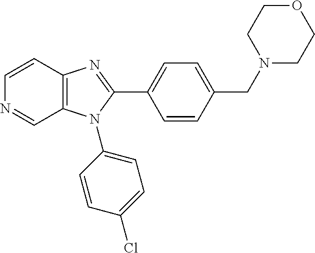 [SMILES: Clc1ccc(-n2c(-c3ccc(CN4CCOCC4)cc3)nc3ccncc32)cc1] | 4-({4-[3-(4-Chlorophenyl)- 3H-imidazo[4,5-c]pyridin- 2-yl]phenyl}methyl) morpholine | Yellow solid Yield 9.81 mg, 2.84% HRMS (ESI+) calcd for [MH]+ of C23H21ClN4O 405.1482 found 405.1478. HPLC: Rt 3.85 min, 100% purity |
| 50 | 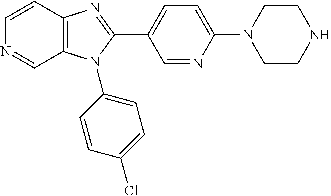 [SMILES: Clc1ccc(-n2c(-c3ccc(N4CCNCC4)nc3)nc3ccncc32)cc1] | 1-{5-[3-(4-Chlorophenyl)- 3H-imidazo[4,5-c]pyridin- 2-yl]pyridin-2- yl}piperazine | Yellow solid Yield 3.10 mg, 1.39% * HRMS (ESI+) calcd for [MH]+ of C21H19ClN6 391.1438 found 391.1427. HPLC: Rt 3.51 min, 100% purity |
| 51 | 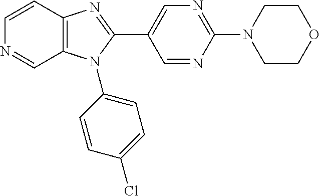 [SMILES: Clc1ccc(-n2c(-c3cnc(N4CCOCC4)nc3)nc3ccncc32)cc1] | 4-{5-[3-(4-Chlorophenyl)- 3H-imidazo[4,5-c]pyridin- 2-yl]pyrimidin-2- yl}morpholine | White solid 2.75 mg, 0.73% HRMS (ESI+) calcd for [MH]+ of C20H17ClN6O 393.1230 found 393.1234. HPLC: Rt 5.06 min, 97.9% purity |
| 52 | 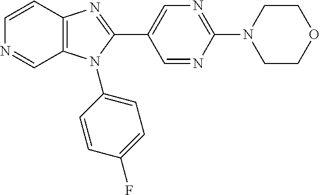 [SMILES: Fc1ccc(-n2c(-c3cnc(N4CCOCC4)nc3)nc3ccncc32)cc1] | 4-{5-[3-(4-Fluorophenyl)- 3H-imidazo[4,5-c]pyridin- 2-yl]pyrimidin-2- yl}morpholine | White solid Yield 166 mg, 36.6% HRMS (ESI+) calcd for [MH]+ of C20H17FN6O 377.1526 found 377.1514. HPLC: Rt 4.68 min, 98.1% purity |
| 53 | 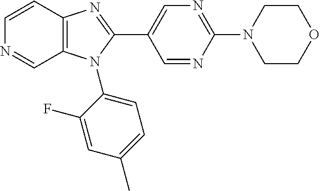 [SMILES: Cc1ccc(-n2c(-c3cnc(N4CCOCC4)nc3)nc3ccncc32)c(F)c1] | 4-{5-[3-(2-Fluoro-4- methylphenyl)-3H- imidazo[4,5-c]pyridin-2- yl]pyrimidin-2- yl}morpholine | White solid Yield 40.1 mg, 13.5% HRMS (ESI+) calcd for [MH]+ of C21H19FN6O 391.1682 found 391.1674. HPLC: Rt 4.77 min, 99.3% purity |
| 54 | 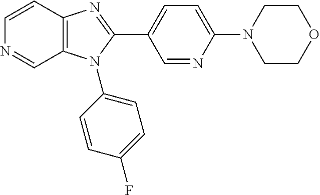 [SMILES: Fc1ccc(-n2c(-c3ccc(N4CCOCC4)nc3)nc3ccncc32)cc1] | 4-{5-[3-(4-Fluorophenyl)- 3H-imidazo[4,5-c]pyridin- 2-yl]pyridin-2- yl}morpholine (Compound 4) | White solid Yield 42.1 mg, 14.0% HRMS (ESI+) calcd for [MH]+ of C21H18FN5O 376.1573 found 376.1560. HPLC: Rt 4.20 min, 98.2% purity |
| 55 | 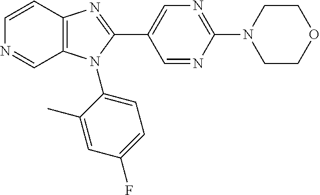 [SMILES: Cc1cc(F)ccc1-n1c(-c2cnc(N3CCOCC3)nc2)nc2ccncc21] | 4-{5-[3-(4-Fluoro-2- methylphenyl)-3H- imidazo[4,5-c]pyridin-2- yl]pyrimidin-2- yl}morpholine | Pale yellow solid Yield 22.2 mg, 3.74% HRMS (ESI+) calcd for [MH]+ of C21H19FN6O 391.1682 found 391.1679. HPLC: Rt 4.67 min, 98.2% purity |
| 56 | 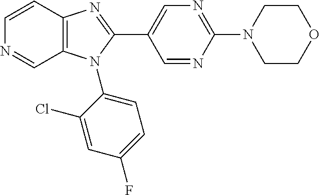 [SMILES: Fc1ccc(-n2c(-c3cnc(N4CCOCC4)nc3)nc3ccncc32)c(Cl)c1] | 4-{5-[3-(2-Chloro-4- fluorophenyl)-3H- imidazo[4,5-c]pyridin-2- yl]pyrimidin-2- yl}morpholine | Off white solid Yield 32.4 mg, 4.97% HRMS (ESI+) calcd for [MH]+ of C20H16ClFN6O 411.1136 found 411.1127. HPLC: Rt 4.72 min, 100% purity |
| 57 | 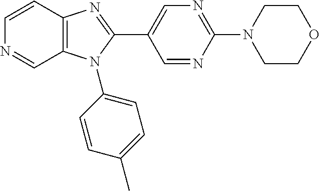 [SMILES: Cc1ccc(-n2c(-c3cnc(N4CCOCC4)nc3)nc3ccncc32)cc1] | 4-{5-[3-(4-Methylphenyl)- 3H-imidazo[4,5-c]pyridin- 2-yl]pyrimidin-2- yl}morpholine | White solid Yield 35.7 mg, 5.22% HRMS (ESI+) calcd for [MH]+ of C21H20N6O 373.1777 found 373.1763. HPLC: Rt 4.80 min, 99.4% purity |
| 58 | 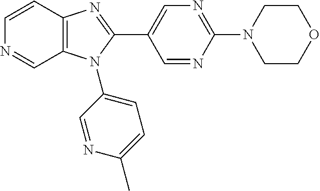 [SMILES: Cc1ccc(-n2c(-c3cnc(N4CCOCC4)nc3)nc3ccncc32)cn1] | 4-{5-[3-(6-Methylpyridin-3- yl)-3H-imidazo[4,5-c] pyridin-2-yl]pyrimidin-2- yl}morpholine | Off white solid Yield 39.0 mg, 5.14% HRMS (ESI+) calcd for [MH]+ of C20H19N7O 374.1729 found 374.1736. HPLC: Rt 3.90 min, 99.1% purity |
| 59 | 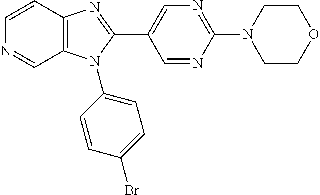 [SMILES: Brc1ccc(-n2c(-c3cnc(N4CCOCC4)nc3)nc3ccncc32)cc1] | 4-{5-[3-(4-Bromophenyl)- 3H-imidazo[4,5-c]pyridin- 2-yl]pyrimidin-2- yl}morpholine | White solid Yield 55.0 mg, 7.80% HRMS (ESI+) calcd for [MH]+ of C20H17BrN6O 437.0725 found 437.0717. HPLC: Rt 5.15 min, 99.0% purity |
| 60 | 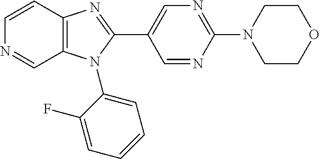 [SMILES: Fc1ccccc1-n1c(-c2cnc(N3CCOCC3)nc2)nc2ccncc21] | 4-{5-[3-(2-Fluorophenyl)- 3H-imidazo[4,5-c]pyridin- 2-yl]pyrimidin-2- yl}morpholine | White solid Yield 18.0 mg, 2.38% HRMS (ESI+) calcd for [MH]+ of C20H17FN6O 377.1526 found 377.1515. HPLC: Rt 4.54 min, 99.4% purity |
| 61 | 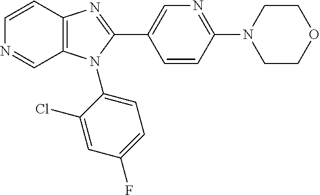 [SMILES: Fc1ccc(-n2c(-c3ccc(N4CCOCC4)nc3)nc3ccncc32)c(Cl)c1] | 4-{5-[3-(2-Chloro-4- fluorophenyl)-3H- imidazo[4,5-c]pyridin-2- yl]pyridin-2-yl}morpholine | Pale yellow solid Yield 23.0 mg, 3.54% HRMS (ESI+) calcd for [MH]+ of C21H17ClFN5O 410.1184 found 410.1189. HPLC: Rt 4.54 min, 99.2% purity |
| 62 | 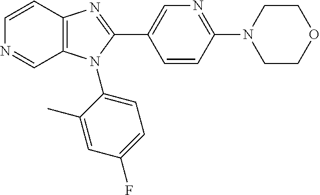 [SMILES: Cc1cc(F)ccc1-n1c(-c2ccc(N3CCOCC3)nc2)nc2ccncc21] | 4-{5-[3-(4-Fluoro-2- methylphenyl)-3H- imidazo[4,5-c]pyridin-2- yl]pyridin-2-yl}morpholine | Pale yellow solid Yield 21.1 mg, 3.57% HRMS (ESI+) calcd for [MH]+ of C22H20FN5O 390.1730 found 390.1723. HPLC: Rt 4.50 min, 99.3% purity |
| 63 | 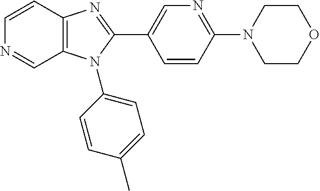 [SMILES: Cc1ccc(-n2c(-c3ccc(N4CCOCC4)nc3)nc3ccncc32)cc1] | 4-{5-[3-(4-Methylphenyl)- 3H-imidazo[4,5-c]pyridin- 2-yl]pyridin-2- yl}morpholine | White solid Yield 72.0 mg, 9.51% HRMS (ESI+) calcd for [MH]+ of C22H21N5O 372.1824 found 372.1820 HPLC: Rt 4.75 min, 100% purity |
| 64 | 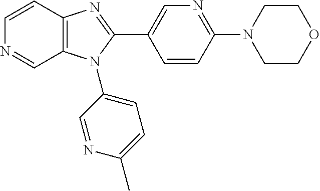 [SMILES: Cc1ccc(-n2c(-c3ccc(N4CCOCC4)nc3)nc3ccncc32)cn1] | 4-{5-[3-(6-Methylpyridin-3- yl)-3H-imidazo[4,5- c]pyridin-2-yl]pyridin-2- yl}morpholine | White solid Yield 52.0 mg, 7.24% HRMS (ESI+) calcd for [MH]+ of C21H20N6O 373.1777 found 373.1768. HPLC: Rt 3.69 min, 100% purity |
| 65 | 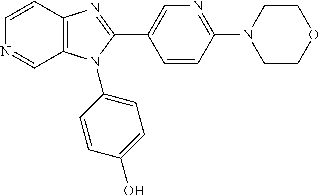 [SMILES: Oc1ccc(-n2c(-c3ccc(N4CCOCC4)nc3)nc3ccncc32)cc1] | 4-{2-[6-Morpholin-4- yl)pyridin-3-yl]-3H- imidazo[4,5-c]pyridin-3- yl}phenol | Off white solid Yield 106 mg, 14.0% HRMS (ESI+) calcd for [MH]+ of C21H19N5O2 374.1617 found 374.1618. HPLC: Rt 3.88 min, 99.5 purity |
| 66 | 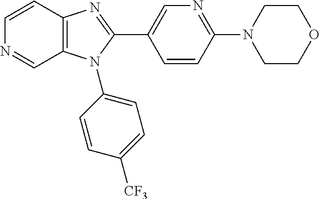 [SMILES: FC(F)(F)c1ccc(-n2c(-c3ccc(N4CCOCC4)nc3)nc3ccncc32)cc1] | 4-(5-{3-[4- (Trifluoromethyl)phenyl]- 3H-imidazo[4,5-c]pyridin- 2-yl}pyridin-2- yl)morpholine | Off white solid Yield 22.0 mg, 3.09% HRMS (ESI+) calcd for [MH]+ of C22H18F3N5O 426.1541 found 426.1549. HPLC: Rt 4.88 min, 99.5% purity |
| 67 | 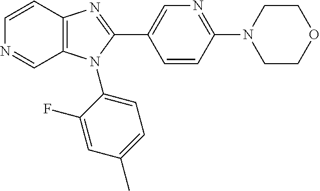 [SMILES: Cc1ccc(-n2c(-c3ccc(N4CCOCC4)nc3)nc3ccncc32)c(F)c1] | 4-{5-[3-(2-Fluoro-4- methylphenyl)-3H- imidazo[4,5-c]pyridin-2- yl]pyridin-2-yl}morpholine | Pale yellow solid Yield 78.3 mg, 10.6% HRMS (ESI+) calcd for [MH]+ of C22H20FN5O 390.1730 found 390.1729. HPLC: Rt 4.66 min, 99.7% purity |
| 68 | 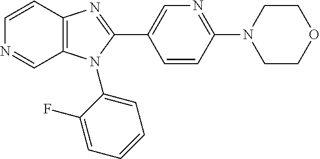 [SMILES: Fc1ccccc1-n1c(-c2ccc(N3CCOCC3)nc2)nc2ccncc21] | 4-{5-[3-(2-Fluorophenyl)- 3H-imidazo[4,5-c]pyridin- 2-yl]pyridin-2- yl}morpholine | White solid Yield 73.0 mg, 9.69% HRMS (ESI+) calcd for [MH]+ of C21H18FN5O 376.1573 found 376.1584. HPLC: Rt 4.33 min, 99.5% purity |
| 69 | 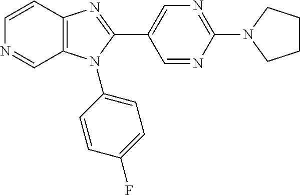 [SMILES: Fc1ccc(-n2c(-c3cnc(N4CCCC4)nc3)nc3ccncc32)cc1] | 5-[3-(4-Fluorophenyl)-3H- imidazo[4,5-c]pyridin-2- yl]-2-(pyrrolidin-1- yl)pyrimidine | White solid Yield 42.2 mg, 7.29% HRMS (ESI+) calcd for [MH]+ of C20H17FN6 361.1577 found 361.1584. HPLC: Rt 4.83 min, 98.5% purity |
| 70 | 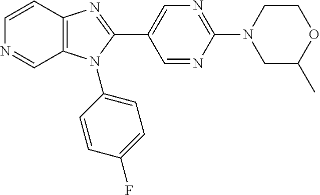 [SMILES: CC1CN(c2ncc(-c3nc4ccncc4n3-c3ccc(F)cc3)cn2)CCO1] | 4-{5-[3-(4-Fluorophenyl)- 3H-imidazo[4,5-c]pyridin- 2-yl]pyridin-2-yl}-2- methylmorpholine | Orange solid Yield 42.7 mg, 9.11% HRMS (ESI+) calcd for [MH]+ of C22H20FN5O 390.1730 found 390.1726. HPLC: Rt 4.54 min, 99.4% purity |
| 71 | 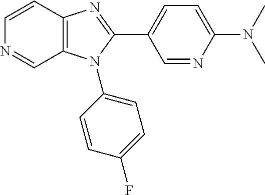 [SMILES: CN(C)c1ccc(-c2nc3ccncc3n2-c2ccc(F)cc2)cn1] | 5-[3-(4-Fluorophenyl)-3H- imidazo[4,5-c]pyridin-2- yl]-N,N-dimethylpyridin-2- amine | White solid Yield 84.0 mg, 12.6% HRMS (ESI+) calcd for [MH]+ of C19H16FN5 334.1468 found 334.1475. HPLC: Rt 3.72 min, 100% purity |
| 72 | 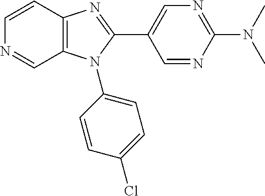 [SMILES: CN(C)c1ncc(-c2nc3ccncc3n2-c2ccc(Cl)cc2)cn1] | 5-[3-(4-Chlorophenyl)-3H- imidazo[4,5-c]pyridin-2- yl]-N,N-dimethylpyrimidin- 2-amine | White solid Yield 12.6 mg, 1.91% HRMS (ESI+) calcd for [MH]+ of C18H15ClN6 351.1125 found 351.1125. HPLC: Rt 4.90 min, 100% purity |
| 74 | 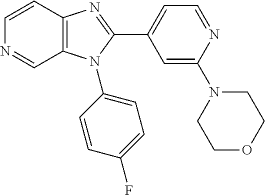 [SMILES: Fc1ccc(-n2c(-c3ccnc(N4CCOCC4)c3)nc3ccncc32)cc1] | 4-{4-[3-(4-Fluorophenyl)- 3H-imidazo[4,5-c]pyridin- 2-yl]pyridin-2- yl}morpholine | white solid Yield 101 mg, 21.7% HRMS (ESI⁺) calcd for [MH]⁺ of C₂₁H₁₈FN₅O 376.1573 found 376.1573. HPLC: Rt 3.89 min, 97.8% purity |
| 75 | 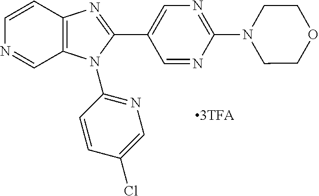 [SMILES: Clc1ccc(-n2c(-c3cnc(N4CCOCC4)nc3)nc3ccncc32)nc1 ; "·3TFA"] | 4-{5-[3-(5-Chloropyridin-2- yl)-3H-imidazo[4,5- c]pyridin-2-yl]pyrimidin-2- yl}morpholine; tris(trifluoroacetic acid) | White solid Yield 29.0 mg, 4.47% HRMS (ESI+) calcd for [MH]+ of C19H16ClN7O 394.1183 found 394.1168. HPLC: Rt 4.68 min, 97.8% purity |
| 76 | 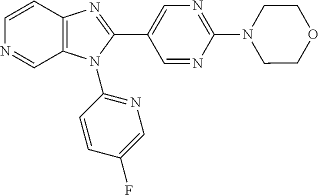 [SMILES: Fc1ccc(-n2c(-c3cnc(N4CCOCC4)nc3)nc3ccncc32)nc1] | 4-{5-[3-(5-Fluoropyridin-2- yl)-3H-imidazo[4,5- c]pyridin-2-yl]pyrimidin-2- yl}morpholine | White solid Yield 53.2 mg, 7.49% HRMS (ESI+) calcd for [MH]+ of C19H16FN7O 378.1479 found 378.1473. HPLC: Rt 4.36 min, 98.4% purity |
| 77 | 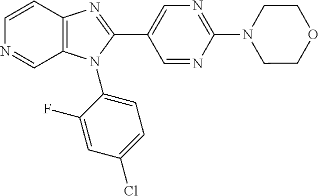 [SMILES: Fc1cc(Cl)ccc1-n1c(-c2cnc(N3CCOCC3)nc2)nc2ccncc21] | 4-{5-[3-(4-Chloro-2- fluorophenyl)-3H- imidazo[4,5-c]pyridin-2- yl]pyrimidin-2- yl}morpholine | Yellow solid Yield 186 mg, 15.4% HRMS (ESI+) calcd for [MH]+ of C20H16ClFN6O 411.1136 found 411.1142. HPLC: Rt 5.09 min, 97.6% purity |
| 78 | 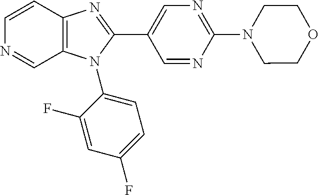 [SMILES: Fc1ccc(-n2c(-c3cnc(N4CCOCC4)nc3)nc3ccncc32)c(F)c1] | 4-{5-[3-(2,4- Difluorophenyl)-3H- imidazo[4,5-c]pyridin-2- yl]pyrimidin-2- yl}morpholine | Pale yellow solid Yield 39.2 mg, 4.54% HRMS (ESI+) calcd for [MH]+ of C20H16F2N6O 395.1432 found 395.1436. HPLC: Rt 4.76 min, 99.6% purity |
| 79 | 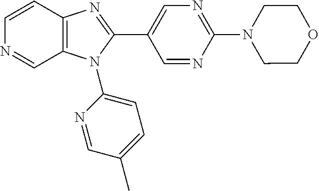 [SMILES: Cc1ccc(-n2c(-c3cnc(N4CCOCC4)nc3)nc3ccncc32)nc1] | 4-{5-[3-(5-Methylpyridin-2- yl)-3H-imidazo[4,5- c]pyridin-2-yl]pyrimidin-2- yl}morpholine | Off white solid Yield 46.1 mg, 9.47% HRMS (ESI+) calcd for [MH]+ of C20H19N7O 374.1729 found 374.1736. HPLC: Rt 4.30 min, 99.6% purity |
| 80 | 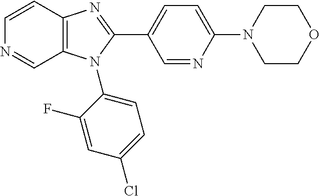 [SMILES: Fc1cc(Cl)ccc1-n1c(-c2ccc(N3CCOCC3)nc2)nc2ccncc21] | 4-{5-[3-(4-Chloro-2- fluorophenyl)-3H- imidazo[4,5-c]pyridin-2- yl]pyridin-2-yl}morpholine | White solid Yield 41.0 mg, 12.8% HRMS (ESI+) calcd for [MH]+ of C21H17ClFN5O 410.1184 found 410.1187. HPLC: Rt 4.58 min, 99.6% purity |
| 81 | 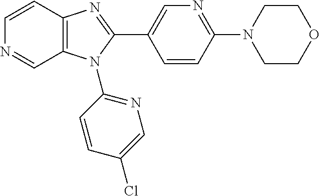 [SMILES: Clc1ccc(-n2c(-c3ccc(N4CCOCC4)nc3)nc3ccncc32)nc1] | 4-{5-[3-(5-Chloropyridin-2- yl)-3H-imidazo[4,5- c]pyridin-2-yl]pyridin-2- yl}morpholine | White solid Yield 7.05 mg, 2.57% HRMS (ESI+) calcd for [MH]+ of C20H17ClN6O 393.1230 found 393.1226. HPLC: Rt 4.45 min, 97.9% purity |
| 82 | 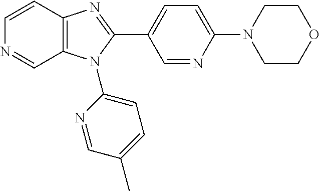 [SMILES: Cc1ccc(-n2c(-c3ccc(N4CCOCC4)nc3)nc3ccncc32)nc1] | 4-{5-[3-(5-Methylpyridin-2- yl)-3H-imidazo[4,5- c]pyridin-2-yl]pyridin-2- yl}morpholine | White solid Yield 38.0 mg, 7.83% HRMS (ESI+) calcd for [MH]+ of C21H20N6O 373.1777 found 373.1787. HPLC: Rt 4.03 min, 99.4% purity |
| 83 | 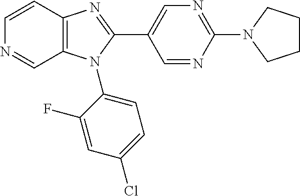 [SMILES: Fc1cc(Cl)ccc1-n1c(-c2cnc(N3CCCC3)nc2)nc2ccncc21] | 5-[3-(4-Chloro-2- fluorophenyl)-3H- imidazo[4,5-c]pyridin-2- yl]-2-(pyrrolidin-1- yl)pyrimidine | White solid Yield 8.20 mg, 2.22% HRMS (ESI+) calcd for [MH]+ of C20H16ClFN6 395.1187 found 395.1190. HPLC: Rt 5.09 min, 100% purity |
| 84 | 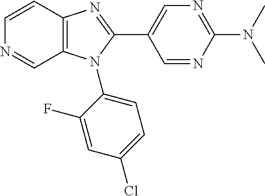 [SMILES: CN(C)c1ncc(-c2nc3ccncc3n2-c2ccc(Cl)cc2F)cn1] | 5-[3-(4-Chloro-2- fluorophenyl)-3H- imidazo[4,5-c]pyridin-2- yl]-N,N-dimethylpyrimidin- 2-amine | Light yellow solid Yield 63.7 mg, 13.6% HRMS (ESI+) calcd for [MH]+ of C18H14ClFN6 369.1031 found 369.1031. HPLC: Rt 4.93 min, 100% purity |
| 85 | 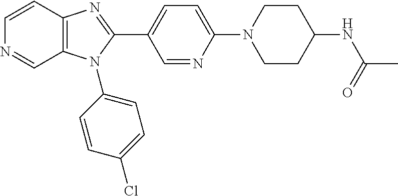 [SMILES: CC(=O)NC1CCN(c2ccc(-c3nc4ccncc4n3-c3ccc(Cl)cc3)cn2)CC1] | N-(1-{5-[3-(4- Chlorophenyl)-3H- imidazo[4,5-c]pyridin-2- yl]pyridin-2-yl}piperidin-4- yl)acetamide | light yellow solid Yield 61.6 mg, 55.8% HRMS (ESI⁺) calcd for [MH]⁺ of C₂₄H₂₃ClN₆O 447.1700 found 447.1701. HPLC: Rt 3.98 min, 99.7% purity |
| 86 | 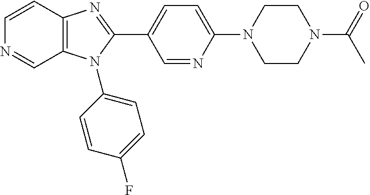 [SMILES: CC(=O)N1CCN(c2ccc(-c3nc4ccncc4n3-c3ccc(F)cc3)cn2)CC1] | 1-(4-{5-[3-(4- Fluorophenyl)-3H- imidazo[4,5-c]pyridin-2- yl]pyridin-2-yl}piperazin-1- yl)ethan-1-one (Compound 2) | white solid Yield 227 mg, 38.7% HRMS (ESI⁺) calcd for [MH]⁺ of C₂₃H₂₁FN₆O 417.1839 found 417.1851. HPLC: Rt 4.26 min, 100% purity. |
| 87 | 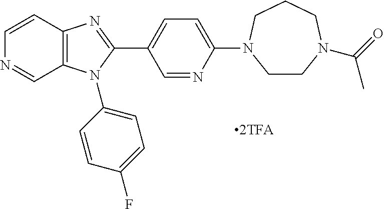 [SMILES: CC(=O)N1CCCN(c2ccc(-c3nc4ccncc4n3-c3ccc(F)cc3)cn2)CC1 ; "·2TFA"] | 1-(4-{5-[3-(4- Fluorophenyl)-3H- imidazo[4,5-c]pyridin-2- yl]pyridin-2-yl}-1,4- diazepan-1-yl)ethan-1- one; bis(trifluoroacetic acid) | pink gum Yield 143 mg, 41.2% HRMS (ESI⁺) calcd for [MH]⁺ of C₂₄H₂₃FN₆O 431.1996 found 431.1997. HPLC: Rt 4.41 min, 99.7% purity |
| 88 | 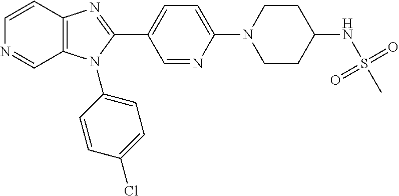 [SMILES: CS(=O)(=O)NC1CCN(c2ccc(-c3nc4ccncc4n3-c3ccc(Cl)cc3)cn2)CC1] | N-(1-{5-[3-(4- Chlorophenyl)-3H- imidazo[4,5-c]pyridin-2- yl]pyridin-2-yl}piperidin-4- yl)methanesulfonamide | yellow solid Yield 30.6 mg, 25.7% HRMS (ESI⁺) calcd for [MH]⁺ of C₂₃H₂₃ClN₆O₂S 483.1370 found 483.1375. HPLC: Rt 4.18 min, 99.4% purity. |
| 89 | 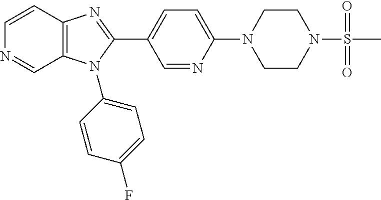 [SMILES: CS(=O)(=O)N1CCN(c2ccc(-c3nc4ccncc4n3-c3ccc(F)cc3)cn2)CC1] | 1-{5-[3-(4-Fluorophenyl)- 3H-imidazo[4,5-c]pyridin- 2-yl]pyridin-2-yl}-4- methanesulfonylpiperazine (compound 3) | yellow solid Yield 44.5 mg, 10.3% HRMS (ESI⁺) calcd for [MH]⁺ of C₂₂H₂₁FN₆O₂S 453.1509 found 453.1522. HPLC: Rt 4.59 min, 98.2% purity |
| 90 | 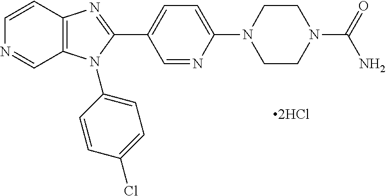 [SMILES: NC(=O)N1CCN(c2ccc(-c3nc4ccncc4n3-c3ccc(Cl)cc3)cn2)CC1 ; "·2HCl"] | 4-{5-[3-(4-Chlorophenyl)- 3H-imidazo[4,5-c]pyridin- 2-yl]pyridin-2- yl}piperazine-1- carboxamide dihydrochloride | yellow solid Yield 21.4 mg, 22.3% HRMS (ESI⁺) calcd for [MH]⁺ of C₂₂H₂₀ClN₇O 434.1496 found 434.1497. HPLC: Rt 4.19 min, 98.5% purity. |
| 91 | 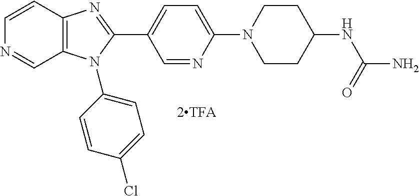 [SMILES: NC(=O)NC1CCN(c2ccc(-c3nc4ccncc4n3-c3ccc(Cl)cc3)cn2)CC1 ; "2·TFA"] | (1-{5-[3-(4-Chlorophenyl)- 3H-imidazo[4,5-c]pyridin- 2-yl]pyridin-2-yl}piperidin- 4-yl)urea; bis(trifluoroacetic acid) | Yellow solid Yield 36.3 mg, 21.7% HRMS (ESI+) calcd for [MH]+ of C23H22ClN7O 448.1653 found 448.1656. HPLC: Rt 3.75 min, 98.7% purity |
| 92 | 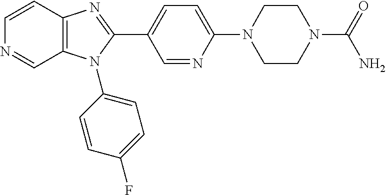 [SMILES: NC(=O)N1CCN(c2ccc(-c3nc4ccncc4n3-c3ccc(F)cc3)cn2)CC1] | 4-{5-[3-(4-Fluorophenyl)- 3H-imidazo[4,5-c]pyridin- 2-yl]pyridin-2- yl}piperazine-1- carboxamide | White solid Yield 44.0 mg, 11.1% HRMS (ESI+) calcd for [MH]+ of C22H20FN7O 418.1791 found 418.1795. HPLC: Rt 3.88 min, 100% purity |
| 93 | 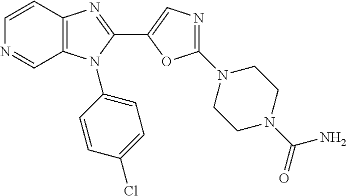 [SMILES: NC(=O)N1CCN(c2ncc(-c3nc4ccncc4n3-c3ccc(Cl)cc3)o2)CC1] | 4-{5-[3-(4-Chlorophenyl)- 3H-imidazo[4,5-c]pyridin- 2-yl]-1,3-oxazol-2- yl}piperazine-1- carboxamide | Pale yellow solid Yield 7.20 mg, 7.71% HRMS (ESI+) calcd for [MH]+ of C20H18ClN7O2 424.1289 found 424.1288. HPLC: Rt 4.20 min, 99.7% purity |
| 94 | 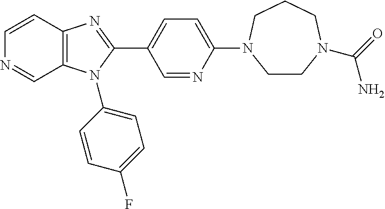 [SMILES: NC(=O)N1CCCN(c2ccc(-c3nc4ccncc4n3-c3ccc(F)cc3)cn2)CC1] | 4-{5-[3-(4-Fluorophenyl)- 3H-imidazo[4,5-c]pyridin- 2-y]pyridin-2-yl}-1,4- diazepane-1-carboxamide | Pink solid Yield 56.7 mg, 24.9% HRMS (ESI+) calcd for [MH]+ of C23H22FN7O 432.1948 found 432.1955. HPLC: Rt 3.83 min, 99.0% purity |
| 95 | 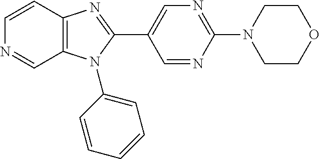 [SMILES: c1ccc(-n2c(-c3cnc(N4CCOCC4)nc3)nc3ccncc32)cc1] | 4-(5-{3-Phenyl-3H- imidazo[4,5-c]pyridin-2- yl}pyrimidin-2- yl)morpholine | white solid Yield 40.2 mg, 38.3% HRMS (ESI⁺) calcd for [MH]⁺ of C₂₀H₁₈N₆O 359.1620 found 359.1613. HPLC: Rt 4.65 min, 99.8% purity |
| 96 | 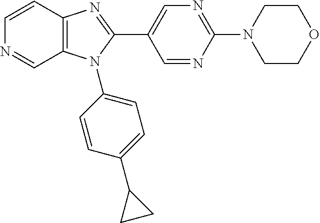 [SMILES: c1cc2nc(-c3cnc(N4CCOCC4)nc3)n(-c3ccc(C4CC4)cc3)c2cn1] | 4-{5-[3-(4- Cyclopropylphenyl)-3H- imidazo[4,5-c]pyridin-2- yl]pyrimidin-2- yl}morpholine | white solid Yield 17.1 mg, 6.74% HRMS (ESI⁺) calcd for [MH]⁺ of C₂₃H₂₂N₆O 399.1933 found 399.1938. HPLC: Rt 5.16 min, 99.4% purity. |
| 97 | 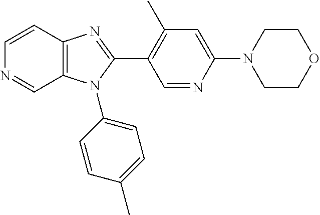 [SMILES: Cc1ccc(-n2c(-c3cnc(N4CCOCC4)cc3C)nc3ccncc32)cc1] | 4-{4-Methyl-5-[3-(4- methylphenyl)-3H- imidazo[4,5-c]pyridin-2- yl]pyridin-2-yl}morpholine | colourless gum Yield 16.4 mg, 2.81% HRMS (ESI⁺) calcd for [MH]⁺ of C₂₃H₂₃N₅O 386.1981 found 386.1982. HPLC: Rt 4.17 min, 99.3% purity |
| 98 | 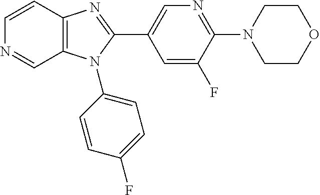 [SMILES: Fc1ccc(-n2c(-c3cnc(N4CCOCC4)c(F)c3)nc3ccncc32)cc1] | 4-{3-Fluoro-5-[3-(4- fluorophenyl)-3H- imidazo[4,5-c]pyridin-2- yl]pyridin-2-yl}morpholine | White solid Yield 134 mg, 27.7% HRMS (ESI+) calcd for [MH]+ of C21H17F2N5O 394.1479 found 394.1478. HPLC: Rt 4.80 min, 99.6% purity |
| 99 | 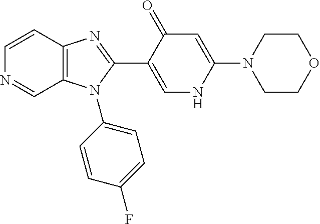 [SMILES: O=c1cc(N2CCOCC2)[nH]cc1-c1nc2ccncc2n1-c1ccc(F)cc1] | 5-[3-(4-Fluorophenyl)-3H- imidazo[4,5-c]pyridin-2- yl]-2-(morpholin-4-yl)-1,4- dihydropyridin-4-one | White solid Yield 15.0 mg, 1.11% HRMS (ESI+) calcd for [MH]+ of C21H18FN5O2 392.1523 found 392.1520. HPLC: Rt 3.73 min, 100% purity |
| 100 | 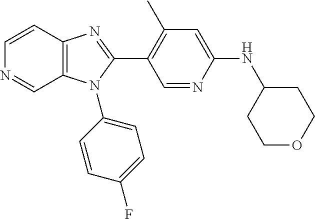 [SMILES: Cc1cc(NC2CCOCC2)ncc1-c1nc2ccncc2n1-c1ccc(F)cc1] | 5-[3-(4-Fluorophenyl)-3H- imidazo[4,5-c]pyridin-2- yl]-4-methyl-N-(oxan-4- yl)pyridin-2-amine | White solid Yield 26.0 mg, 8.77% HRMS (ESI+) calcd for [MH]+ of C23H22FN5O 404.1887 found 404.1892. HPLC: Rt 3.56 min, 100% purity |
| 101 | 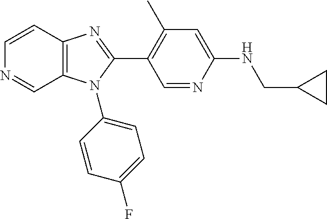 [SMILES: Cc1cc(NCC2CC2)ncc1-c1nc2ccncc2n1-c1ccc(F)cc1] | N-(Cyclopropylmethyl)-5- [3-(4-fluorophenyl)-3H- imidazo[4,5-c]pyridin-2- yl]-4-methylpyridin-2- amine | White solid Yield 43.0 mg, 15.7% HRMS (ESI+) calcd for [MH]+ of C22H20FN5 374.1781 found 374.1787. HPLC: Rt 3.90 min, 100% purity |
| 102 | 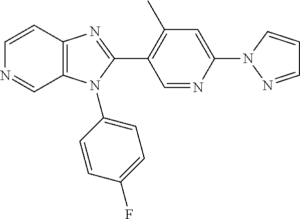 [SMILES: Cc1cc(-n2cccn2)ncc1-c1nc2ccncc2n1-c1ccc(F)cc1] | 5-[3-(4-Fluorophenyl)-3H- imidazo[4,5-c]pyridin-2- yl]-4-methyl-2-(1H- pyrazol-1-yl)pyridine | Off white solid Yield 28.1 mg, 12.9% HRMS (ESI+) calcd for [MH]+ of C21H15FN6 371.1420 found 371.1419. HPLC: Rt 4.98 min, 99.8% purity |
| 103 | 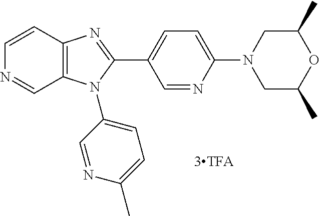 [SMILES: Cc1ccc(-n2c(-c3ccc(N4C[C@@H](C)O[C@@H](C)C4)nc3)nc3ccncc32)cn1 ; "3·TFA"] | (2R,6S)-2,6-Dimethyl-4- {5-[3-(6-methylpyridin-3- yl)-3H-imidazo[4,5- c]pyridin-2-yl]pyridin-2- yl}morpholine; tris(trifluoroacetic acid) | Yellow gum Yield 26.0 mg, 1.75% HRMS (ESI+) calcd for [MH]+ of C23H24N6O 401.2090 found 401.2084. HPLC: Rt 4.27 min, 99.1% purity |
| 104 | 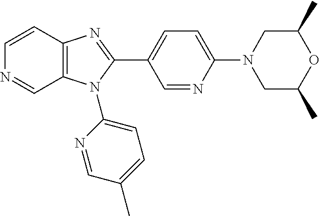 [SMILES: Cc1ccc(-n2c(-c3ccc(N4C[C@@H](C)O[C@@H](C)C4)nc3)nc3ccncc32)nc1] | (2R,6S)-2,6-Dimethyl-4- {5-[3-(5-methylpyridin-2- yl)-3H-imidazo[4,5- c]pyridin-2-yl]pyridin-2- yl}morpholine | White solid Yield 26.0 mg, 3.71% HRMS (ESI+) calcd for [MH]+ of C23H24N6O 401.2090 found 401.2098. HPLC: Rt 4.66 min, 98.9% purity |
| 105 | 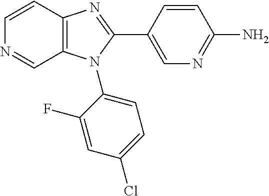 [SMILES: Nc1ccc(-c2nc3ccncc3n2-c2ccc(Cl)cc2F)cn1] | 5-[3-(4-Chloro-2- fluorophenyl)-3H- imidazo[4,5-c]pyridin-2- yl]pyridin-2-amine | Pale pink solid Yield 24.0 mg, 8.49% HRMS (ESI+) calcd for [MH]+ of C17H11ClFN5 340.0765 found 340.0773. HPLC: Rt 3.30 min, 100% purity |
| 106 | 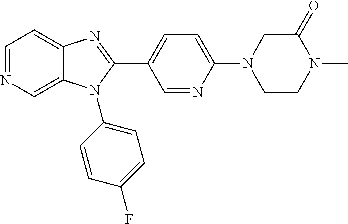 [SMILES: CN1CCN(c2ccc(-c3nc4ccncc4n3-c3ccc(F)cc3)cn2)CC1=O] | 4-{5-[3-(4-Fluorophenyl)- 3H-imidazo[4,5-c]pyridin- 2-yl]pyridin-2-yl}-1- methylpiperazin-2-one | Pale yellow solid Yield 38.5 mg, 13.1% HRMS (ESI+) calcd for [MH]+ of C22H19FN6O 403.1682 found 403.1684. HPLC: Rt 4.07 min, 99.4% purity |
| 107 | 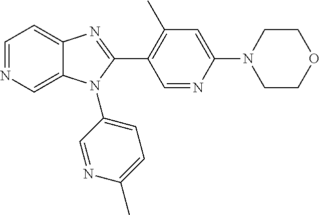 [SMILES: Cc1ccc(-n2c(-c3cnc(N4CCOCC4)cc3C)nc3ccncc32)cn1] | 4-{4-Methyl-5-[3-(6- methylpyridin-3-yl)-3H- imidazo[4,5-c]pyridin-2- yl]pyridin-2-yl}morpholine | Pale yellow gum Yield 24.0 mg, 3.11% LCMS (ES+): 387.0 [MH]+ HPLC: Rt 3.74 min, 98.7% purity |
| 108 | 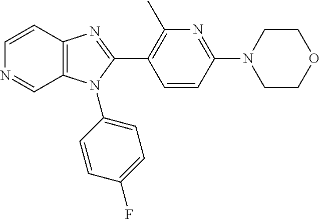 [SMILES: Cc1nc(N2CCOCC2)ccc1-c1nc2ccncc2n1-c1ccc(F)cc1] | 4-{5-[3-(4-Fluorophenyl)- 3H-imidazo[4,5-c]pyridin- 2-yl]-6-methylpyridin-2- yl}morpholine | white solid Yield 47.0 mg, 43.5% HRMS (ESI⁺) calcd for [MH]⁺ of C₂₂H₂₀FN₅O 390.1730 found 390.1737. HPLC: Rt 3.96 min, 99.7% purity |
| 109 | 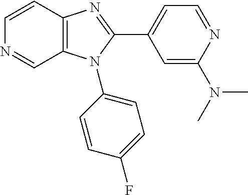 [SMILES: CN(C)c1cc(-c2nc3ccncc3n2-c2ccc(F)cc2)ccn1] | 4-[3-(4-Fluorophenyl)-3H- imidazo[4,5-c]pyridin-2- yl]-N,N-dimethylpyridin-2- amine | From Intermediate 131 Yellow solid Yield 110 mg, 53.4% HRMS (ESI+) calcd for [MH]+ of C19H16FN5 334.1468 found 334.1476. HPLC: Rt 3.19 min, 99.7% purity |
| 110 | 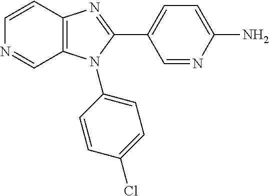 [SMILES: Nc1ccc(-c2nc3ccncc3n2-c2ccc(Cl)cc2)cn1] | 5-[3-(4-Chlorophenyl)-3H- imidazo[4,5-c]pyridin-2- yl]pyridin-2-amine | From Intermediate 128 White solid Yield 20.0 mg, 26.2% HRMS (ESI+) calcd for [MH]+ of C17H12ClN5 322.0859 found 322.0849. HPLC: Rt 3.45 min, 99.7% purity |
| 111 | 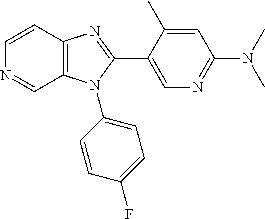 [SMILES: Cc1cc(N(C)C)ncc1-c1nc2ccncc2n1-c1ccc(F)cc1] | 5-[3-(4-Fluorophenyl)-3H- imidazo[4,5-c]pyridin-2- yl]-N,N,4-trimethylpyridin- 2-amine | From Intermediate 133 Light yellow solid Yield 24.8 mg, 29.2% HRMS (ESI+) calcd for [MH]+ of C20H18FN5 348.1624 found 348.1631. HPLC: Rt 3.44 min, 99.1% purity |
| 112 | 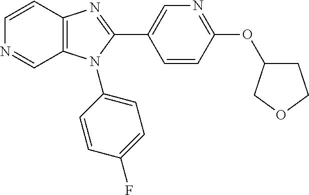 [SMILES: Fc1ccc(-n2c(-c3ccc(OC4CCOC4)nc3)nc3ccncc32)cc1] | 5-[3-(4-Fluorophenyl)-3H- imidazo[4,5-c]pyridin-2- yl]-2-(oxolan-3- yloxy)pyridine | white solid Yield 19.2 mg, 20.7% HRMS (ESI⁺) calcd for [MH]⁺ of C₂₁H₁₇FN₄O₂ 377.1414 found 377.1419. HPLC: Rt: 4.78 min, 98.9% purity |
| 113 | 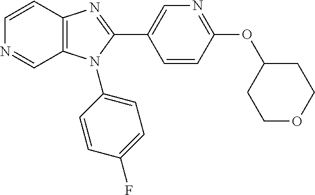 [SMILES: Fc1ccc(-n2c(-c3ccc(OC4CCOCC4)nc3)nc3ccncc32)cc1] | 5-[3-(4-Fluorophenyl)-3H- imidazo[4,5-c]pyridin-2- yl]-2-(oxan-4- yloxy)pyridine | whtie solid Yield 20.0 mg, 20.8% HRMS (ESI⁺) calcd for [MH]⁺ of C₂₂H₁₉FN₄O₂ 391.1570 found 391.1566. HPLC: Rt 5.04 min, 99.7% purity. |
| 114 | 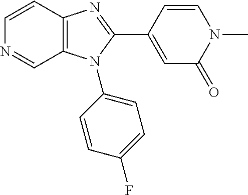 [SMILES: Cn1ccc(-c2nc3ccncc3n2-c2ccc(F)cc2)cc1=O] | 4-[3-(4-Fluorophenyl)-3H- imidazo[4,5-c]pyridin-2- yl]-1-methyl-1,2- dihydropridin-2-one | white solid Yield 1.41 mg, 5.19% HRMS (ESI⁺) calcd for [MH]⁺ of C₁₈H₁₃FN₄O 321.1151 found 321.1156. HPLC: Rt 3.45 min, 98.9% purity |
| 115 | 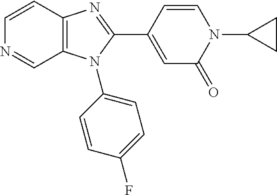 [SMILES: O=c1cc(-c2nc3ccncc3n2-c2ccc(F)cc2)ccn1C1CC1] | 1-Cyclopropyl-4-[3-(4- fluorophenyl)-3H- imidazo[4,5-c]pyridin-2- yl]-1,2-dihydropyridin-2- one | white solid Yield 22.4 mg, 16.5% HRMS (ESI⁺) calcd for [MH]⁺ of C₂₀H₁₅FN₄O 347.1308 found 347.1309. HPLC: Rt 3.88 min, 100% purity |
| 116 | 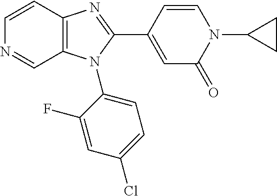 [SMILES: O=c1cc(-c2nc3ccncc3n2-c2ccc(Cl)cc2F)ccn1C1CC1] | 4-[3-(4-Chloro-2- fluorophenyl)-3H- imidazo[4,5-c]pyridin-2- yl]-1-cyclopropyl-1,2- dihdyropyridin-2-one | white solid Yield 31.0 mg, 34.7% HRMS (ESI⁺) calcd for [MH]⁺ of C₂₀H₁₄ClFN₄O 381.0918 found 381.0922. HPLC: Rt 4.13 min, 100% purity |
| 117 | 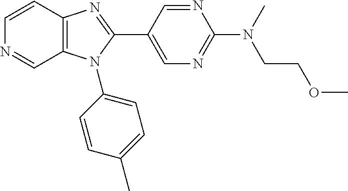 [SMILES: COCCN(C)c1ncc(-c2nc3ccncc3n2-c2ccc(C)cc2)cn1] | N-(2-Methoxyethyl)-N- methyl-5-[3-(4- methylphenyl)-3H- imidazo[4,5-c]pyridin-2- yl]pyrimidin-2-amine | white solid Yield 60.3 mg, 10.6% HRMS (ESI⁺) calcd for [MH]⁺ of C₂₁H₂₂N₆O 375.1933 found 375.1942. HPLC: Rt: 4.99 min, 99.3% |
| 118 | 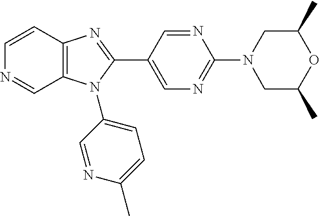 [SMILES: Cc1ccc(-n2c(-c3cnc(N4C[C@@H](C)O[C@@H](C)C4)nc3)nc3ccncc32)cn1] | (2R,6S)-2,6-Dimethyl-4- {5-[3-(6-methylpyridin-3- yl)-3H-imidazo[4,5- c]pyridin-2-yl]pyrimidin-2- yl}morpholine | White solid Yield 58.0 mg, 7.23% HRMS (ESI+) calcd for [MH]+ of C22H23N7O 402.2042 found 402.2046. HPLC: Rt 4.52 min, 97.6% purity |
| 119 | 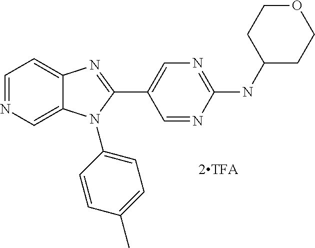 [SMILES: Cc1ccc(-n2c(-c3cnc(NC4CCOCC4)nc3)nc3ccncc32)cc1 ; "2·TFA"] | 5-[3-(4-Methylphenyl)-3H- imidazo[4,5-c]pyridin-2- yl]-N-(oxan-4-yl)pyrimidin- 2-amine; bis(trifluoroacetic acid) | White solid Yield 12.0 mg, 1.55% HRMS (ESI+) calcd for [MH]+ of C22H22N6O 387.1933 found 387.1936. HPLC: Rt 4.42 min, 99.4% purity |
| 120 | 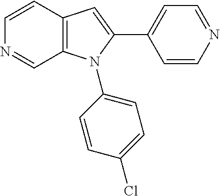 [SMILES: Clc1ccc(-n2c(-c3ccncc3)cc3ccncc32)cc1] | 4-[1-(4-Chlorophenyl)-1H- pyrrrolo[2,3-c]pyridin-2- yl]pyridine | light yellow solid yeild 21.8 mg, 16.3% HRMS (ESI⁺ calcd for [MH]⁺ of C₁₈H₁₂ClN₃ 306.0798 found 306.0809. HPLC: Rt 3.52 min, 99.9% purity |
| 121 | 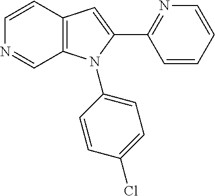 [SMILES: Clc1ccc(-n2c(-c3ccccn3)cc3ccncc32)cc1] | 2-[1-(4-Chlorophenyl)-1H- pyrrolo[2,3-c]pyridin-2- yl]pyridine | Yellow gum Yield 14.0 mg, 6.98% HRMS (ESI+) calcd for [MH]+ of C18H12ClN3 306.0798 found 306.0811. HPLC: Rt 4.82 min, 99.1% purity |
| 122 | 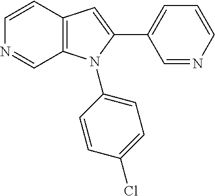 [SMILES: Clc1ccc(-n2c(-c3cccnc3)cc3ccncc32)cc1] | 3-[1-(4-Chlorophenyl)-1H- pyrrolo[2,3-c]pyridin-2- yl]pyridine | Yellow gum Yield 13.1 mg, 9.80% HRMS (ESI+) calcd for [MH]+ of C18H12ClN3 306.0798 found 306.0810. HPLC: Rt 3.95 min, 99.1% purity |
| 123 | 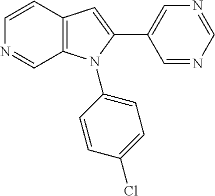 [SMILES: Clc1ccc(-n2c(-c3cncnc3)cc3ccncc32)cc1] | 5-[1-(4-Chlorophenyl)-1H- pyrrolo[2,3-c]pyridin-2- yl]pyrimidine | White solid Yield 64.9 mg, 32.2% HRMS (ESI+) calcd for [MH]+ of C17H11ClN4 307.0750 found 307.0753. HPLC: Rt 4.24 min, 99.1% purity |
| 124 | 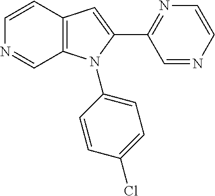 [SMILES: Clc1ccc(-n2c(-c3cnccn3)cc3ccncc32)cc1] | 2-[1-(4-Chlorophenyl)-1H- pyrrolo[2,3-c]pyridin-2- yl]pyrazine | Yellow gum Yield 28.0 mg, 13.9% HRMS (ESI+) calcd for [MH]+ of C17H11ClN4 307.0750 found 307.0764. HPLC: Rt 4.53 min, 99.7% purity |
| 125 | 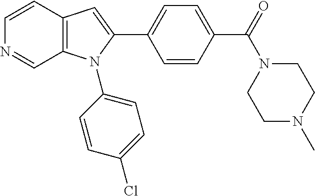 [SMILES: CN1CCN(C(=O)c2ccc(-c3cc4ccncc4n3-c3ccc(Cl)cc3)cc2)CC1] | 1-({4-[1-(4-Chlorophenyl)- 1H-pyrrolo[2,3-c]pyridin-2- yl]phenyl}carbonyl)-4- methylpiperazine | Yellow solid Yield 23.0 mg, 8.14% HRMS (ESI+) calcd for [MH]+ of C25H23ClN4O 431.1638 found 431.1638. HPLC: Rt 3.77 min, 97.9% purity |
| 126 | 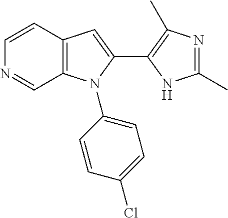 [SMILES: Cc1nc(C)c(-c2cc3ccncc3n2-c2ccc(Cl)cc2)[nH]1] | 5-[1-(4-Chlorophenyl)-1H- pyrrolo[2,3-c]pyridin-2-yl]- 2,4-dimethyl-1H-imidazole | Off white solid Yield 14.6 mg, 6.90% HRMS (ESI+) calcd for [MH]+ of C18H15ClN4 323.1063 found 323.1067. HPLC: Rt 3.45 min, 100% purity |
| 127 | 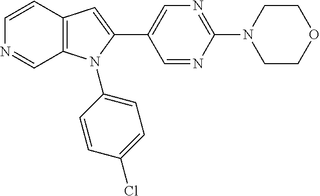 [SMILES: Clc1ccc(-n2c(-c3cnc(N4CCOCC4)nc3)cc3ccncc32)cc1] | 4-{5-[1-(4-Chlorophenyl)- 1H-pyrrolo[2,3-c]pyridin-2- yl]pyrimidin-2- yl}morpholine | Yellow solid Yield 67.2 mg, 19.6% HRMS (ESI+) calcd for [MH]+ of C21H18ClN5O 392.1278 found 392.1286. HPLC: Rt 5.12 min, 100% purity |
| 128 | 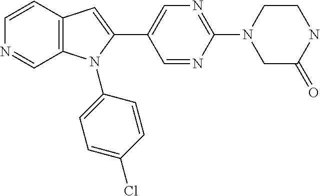 [SMILES: O=C1CN(c2ncc(-c3cc4ccncc4n3-c3ccc(Cl)cc3)cn2)CCN1] | 4-{5-[1-(4-Chlorophenyl)- 1H-pyrrolo[2,3-c]pyridin-2- yl]pyrimidin-2-yl}piperazin- 2-one | White solid Yield 71.5 mg, 16.1% HRMS (ESI+) calcd for [MH]+ of C21H17ClN6O 405.1230 found 405.1226. HPLC: Rt 4.23 min, 98.6% purity. |
| 129 | 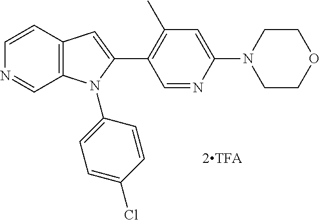 [SMILES: Cc1cc(N2CCOCC2)ncc1-c1cc2ccncc2n1-c1ccc(Cl)cc1 ; "2·TFA"] | 4-{5-[1-(4-Chlorophenyl)- 1H-pyrrolo[2,3-c]pyridin-2- yl]-4-methylpyridin-2- yl}morpholine; bis(trifluoroacetic acid) | Colourless gum Yield 6.55 mg, 0.95% HRMS (ESI+) calcd for [MH]+ of C23H21ClN4O 405.1482 found 405.1494. HPLC: Rt 4.06 min, 98.6% purity. |
| 130 | 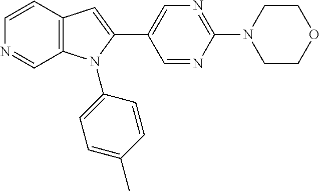 [SMILES: Cc1ccc(-n2c(-c3cnc(N4CCOCC4)nc3)cc3ccncc32)cc1] | 4-{5-[1-(4-Methylphenyl)- 1H-pyrrolo[2,3-c]pyridin-2- yl]pyrimidin-2- yl}morpholine | White solid Yield 80.0 mg, 17.9% HRMS (ESI+) calcd for [MH]+ of C22H21N5O 372.1824 found 372.1828. HPLC: Rt 5.21 min, 100% purity. |
| 131 | 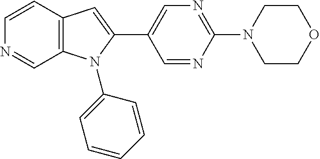 [SMILES: c1ccc(-n2c(-c3cnc(N4CCOCC4)nc3)cc3ccncc32)cc1] | 4-(5-{1-Phenyl-1H- pyrrolo[2,3-c]pyridin-2- yl}pyrimidin-2- yl)morpholine | White solid Yield 58.5 mg, 12.7% HRMS (ESI+) calcd for [MH]+ of C21H19N5O 358.1668 found 358.1685. HPLC: Rt 4.90 min, 97.4% purity. |
| 132 | 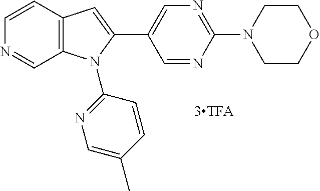 [SMILES: Cc1ccc(-n2c(-c3cnc(N4CCOCC4)nc3)cc3ccncc32)nc1 ; "3·TFA"] | 4-{5-[1-(5-Methylpyridin-2- yl)-1H-pyrrolo[2,3- c]pyridin-2-yl]pyrimidin-2- yl}morpholine; tris(trifluoroacetic acid) | Yellow solid Yield 3.35 mg, 0.87% HRMS (ESI+) calcd for [MH]+ of C21H20N6O 373.1777 found 373.1794. HPLC: Rt 4.68 min, 99.6% purity. |
| 133 | 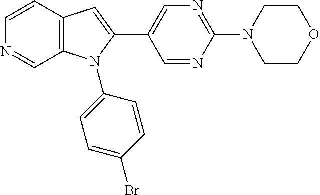 [SMILES: Brc1ccc(-n2c(-c3cnc(N4CCOCC4)nc3)cc3ccncc32)cc1] | 4-{5-[1-(4-Bromophenyl)- 1H-pyrrolo[2,3-c]pyridin-2- yl]pyrimidin-2- yl}morpholine | Light yellow solid Yield 38.0 mg, 9.52% HRMS (ESI+) calcd for [MH]+ of C21H18BrN5O 436.0773 found 436.0773. HPLC: Rt 5.32 min, 96.2% purity. |
| 134 | 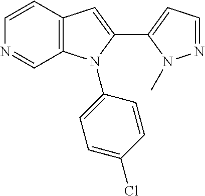 [SMILES: Cn1nccc1-c1cc2ccncc2n1-c1ccc(Cl)cc1] | 5-[1-(4-Chlorophenyl)-1H- pyrrolo[2,3-c]pyridin-2-yl]- 1-methyl-1H-pyrazole | Orange solid Yield 133 mg, 32.8% HRMS (ESI+) calcd for [MH]+ of C17H13ClN4 309.0907 found 309.0918. HPLC: Rt 4.64 min, 98.4% purity. |
| 135 | 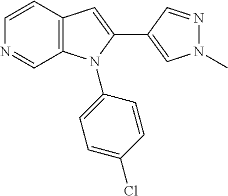 [SMILES: Cn1cc(-c2cc3ccncc3n2-c2ccc(Cl)cc2)cn1] | 4-[1-(4-Chlorophenyl)-1H- pyrrolo[2,3-c]pyridin-2-yl]- 1-methyl-1H-pyrazole | Orange solid Yield 153 mg, 37.9% HRMS (ESI+) calcd for [MH]+ of C17H13ClN4 309.0907 found 309.0910. HPLC: Rt 4.87 min, 99.5% purity. |
| 136 | 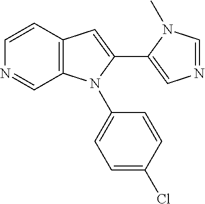 [SMILES: Cn1cncc1-c1cc2ccncc2n1-c1ccc(Cl)cc1] | 5-[1-(4-Chlorophenyl)-1H- pyrrolo[2,3-c]pyridin-2-yl]- 1-methyl-1H-imidazole | White solid Yield 63.8 mg, 15.8% HRMS (ESI+) calcd for [MH]+ of C17H13ClN4 309.0907 found 309.0914. HPLC: Rt 3.43 min, 100% purity. |
| 137 | 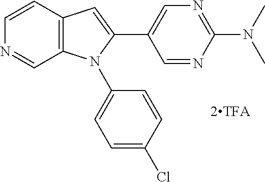 [SMILES: CN(C)c1ncc(-c2cc3ccncc3n2-c2ccc(Cl)cc2)cn1 ; "2·TFA"] | 5-[1-(4-Chlorophenyl)-1H- pyrrolo[2,3-c]pyridin-2-yl]- N,N-dimethylpyrimidin-2- amine; bis(trifluoroacetic acid) | Yellow gum Yield 142 mg, 18.7% HRMS (ESI+) calcd for [MH]+ of C19H16ClN5 350.1172 found 350.1180. HPLC: Rt 5.46 min, 100% purity. |
| 138 | 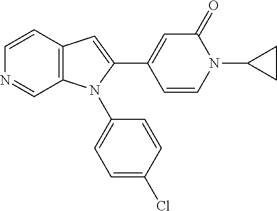 [SMILES: O=c1cc(-c2cc3ccncc3n2-c2ccc(Cl)cc2)ccn1C1CC1] | 4-[1-(4-Chlorophenyl)-1H- pyrrolo[2,3-c]pyridin-2-yl]- 1-cyclopropyl-1,2- dihydropyridin-2-one | Yellow solid Yield 234 mg, 36.9% HRMS (ESI+) calcd for [MH]+ of C21H16ClN3O 362.1060 found 362.1063. HPLC: Rt 4.76 min, 99.0% purity. |
| 139 | 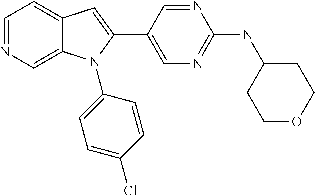 [SMILES: Clc1ccc(-n2c(-c3cnc(NC4CCOCC4)nc3)cc3ccncc32)cc1] | 5-[1-(4-Chlorophenyl)-1H- pyrrolo[2,3-c]pyridin-2-yl]- N-(oxan-4-yl)pyrimidin-2- amine | White solid Yield 122 mg, 22.8% HRMS (ESI+) calcd for [MH]+ of C22H20ClN5O 406.1435 found 406.1435. HPLC: Rt 4.78 min, 99.3% purity. |
| 140 | 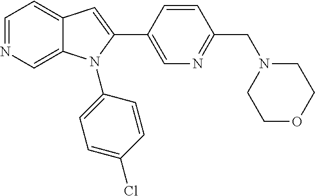 [SMILES: Clc1ccc(-n2c(-c3ccc(CN4CCOCC4)nc3)cc3ccncc32)cc1] | 4-({5-[1-(4-Chlorophenyl)- 1H-pyrrolo[2,3-c]pyridin-2- yl]pyridin-2- yl}methyl)morpholine | Beige solid Yield 29.7 mg, 4.20% HRMS (ESI+) calcd for [MH]+ of C23H21ClN4O 405.1482 found 405.1485. HPLC: Rt 3.91 min, 98.0% purity. |
| 141 | 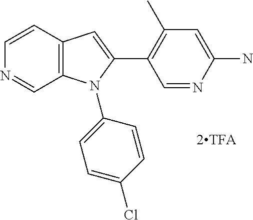 [SMILES: Cc1cc(N)ncc1-c1cc2ccncc2n1-c1ccc(Cl)cc1 ; "2·TFA"] | 5-[1-(4-Chlorophenyl)-1H- pyrrolo[2,3-c]pyridin-2-yl]- 4-methylpyridin-2-amine; bis(trifluoroacetic acid) | Colourless gum Yield 1.68 mg, 0.34% LCMS (ES+): 335.1 [MH]+ HPLC: Rt 3.63 min, 96.8% purity. |
| 142 | 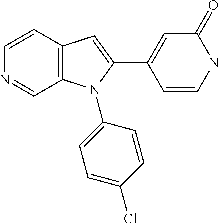 [SMILES: O=c1cc(-c2cc3ccncc3n2-c2ccc(Cl)cc2)cc[nH]1] | 4-[1-(4-Chlorophenyl)-1H- pyrrolo[2,3-c]pyridin-2-yl]- 1,2-dihydropyridin-2-one | Off white solid Yield 52.0 mg, 14.8% HRMS (ESI+) calcd for [MH]+ of C18H12ClN3O 322.0747 found 322.0753. HPLC: Rt 3.75 min, 97.6% purity. |
| 143 | 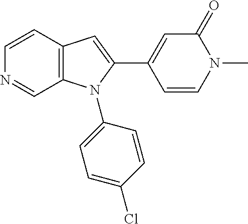 [SMILES: Cn1ccc(-c2cc3ccncc3n2-c2ccc(Cl)cc2)cc1=O] | 4-[1-(4-Chlorophenyl)-1H- pyrrolo[2,3-c]pyridin-2-yl]- 1-methyl-1,2- dihydropyridin-2-one | Off white solid Yield 56.9 mg, 15.5% HRMS (ESI+) calcd for [MH]+ of C19H14ClN3O 336.0904 found 336.0909. HPLC: Rt 4.07 min, 98.6% purity. |
| 144 | 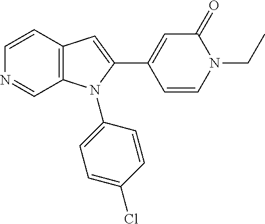 [SMILES: CCn1ccc(-c2cc3ccncc3n2-c2ccc(Cl)cc2)cc1=O] | 4-[1-(4-Chlorophenyl)-1H- pyrrolo[2,3-c]pyridin-2-yl]- 1-ethyl-1,2-dihydropyridin- 2-one | Off white solid Yield 59.5 mg, 15.6% HRMS (ESI+) calcd for [MH]+ of C20H16ClN3O 350.1060 found 350.1065. HPLC: Rt 4.39 min, 100% purity. |
| 145 | 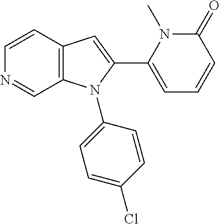 [SMILES: Cn1c(-c2cc3ccncc3n2-c2ccc(Cl)cc2)cccc1=O] | 6-[1-(4-Chlorophenyl)-1H- pyrrolo[2,3-c]pyridin-2-yl]- 1-methyl-1,2- dihydropyridin-2-one | White solid Yield 82.2 mg, 16.0% HRMS (ESI+) calcd for [MH]+ of C19H14ClN3O 336.0904 found 336.0914. HPLC: Rt 4.20 min, 99.5% purity. |
| 146 | 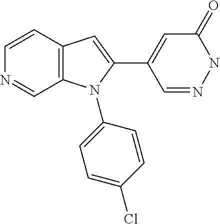 [SMILES: O=c1cc(-c2cc3ccncc3n2-c2ccc(Cl)cc2)cn[nH]1] | 5-[1-(4-Chlorophenyl)-1H- pyrrolo[2,3-c]pyridin-2-yl]- 2,3-dihydropyridazin-3- one | Yellow solid Yield 35.5 mg, 10.1% HRMS (ESI+) calcd for [MH]+ of C17H11ClN4O 323.0699 found 323.0700. HPLC: Rt 3.86 min, 98.4% purity. |
| 147 | 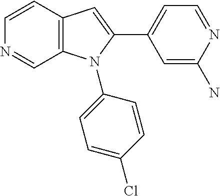 [SMILES: Nc1cc(-c2cc3ccncc3n2-c2ccc(Cl)cc2)ccn1] | 4-[1-(4-Chlorophenyl)-1H- pyrrolo[2,3-c]pyridin-2- yl]pyridin-2-amine | Off white solid Yield 103 mg, 29.4% HRMS (ESI+) calcd for [MH]+ of C18H13ClN4 321.0907 found 321.0901. HPLC: Rt 3.56 min, 100% purity. |
| 148 | 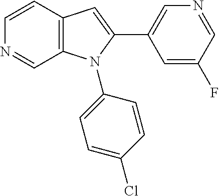 [SMILES: Fc1cncc(-c2cc3ccncc3n2-c2ccc(Cl)cc2)c1] | 3-[1-(4-Chlorophenyl)-1H- pyrrolo[2,3-c]pyridin-2-yl]- 5-fluoropyridine | Yellow solid Yield 21.2 mg, 6.00% HRMS (ESI+) calcd for [MH]+ of C18H11ClFN3 324.0704 found 324.0714. HPLC: Rt 4.87 min, 100% purity. |
| 149 | 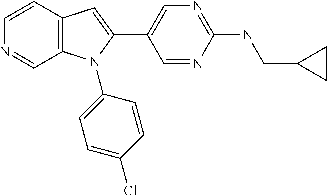 [SMILES: Clc1ccc(-n2c(-c3cnc(NCC4CC4)nc3)cc3ccncc32)cc1] | 5-[1-(4-Chlorophenyl)-1H- pyrrolo[2,3-c]pyridin-2-yl]- N- (cyclopropylmethyl) pyrimidin-2-amine | Off white solid Yield 32.0 mg, 7.79% HRMS (ESI+) calcd for [MH]+ of C21H18ClN5 376.1329 found 376.1326. HPLC: Rt 5.38 min, 99.1% purity. |
| 150 | 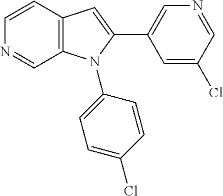 [SMILES: Clc1ccc(-n2c(-c3cncc(Cl)c3)cc3ccncc32)cc1] | 3-Chloro-5-[1-(4- chlorophenyl)-1H- pyrrolo[2,3-c]pyridin-2- yl]pyridine | Off white solid Yield 19.2 mg, 5.16% HRMS (ESI+) calcd for [MH]+ of C18H11Cl2N3 340.0408 found 340.0418. HPLC: Rt 5.17 min, 100% purity. |
| 151 | 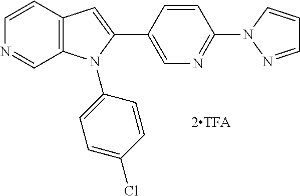 [SMILES: Clc1ccc(-n2c(-c3ccc(-n4cccn4)nc3)cc3ccncc32)cc1 ; "2·TFA"] | 5-[1-(4-Chlorophenyl)-1H- pyrrolo[2,3-c]pyridin-2-yl]- 2-(1H-pyrazol-1- yl)pyridine; bis(trifluoroacetic acid) | Yellow solid Yield 4.51 mg, 0.69% HRMS (ESI+) calcd for [MH]+ of C21H14ClN5 372.1016 found 372.1025. HPLC: Rt 5.57 min, 100% purity. |
| 152 | 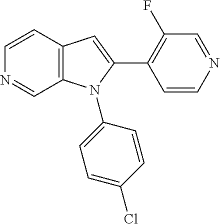 [SMILES: Fc1cnccc1-c1cc2ccncc2n1-c1ccc(Cl)cc1] | 4-[1-(4-Chlorophenyl)-1H- pyrrolo[2,3-c]pyridin-2-yl]- 3-fluoropyridine | White solid Yield 9.24 mg, 2.61% HRMS (ESI+) calcd for [MH]+ of C18H11ClFN3 324.0704 found 324.0710. HPLC: Rt 4.74 min, 100% purity. |
| 153 | 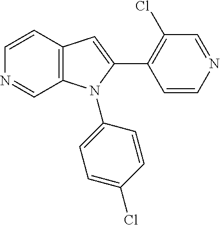 [SMILES: Clc1ccc(-n2c(-c3ccncc3Cl)cc3ccncc32)cc1] | 3-Chloro-4-[1-(4- chlorophenyl)-1H- pyrrolo[2,3-c]pyridin-2- yl]pyridine | White solid Yield 40.1 mg, 9.29% HRMS (ESI+) calcd for [MH]+ of C18H11Cl2N3 340.0408 found 340.0421. HPLC: Rt 4.98 min, 100% purity |
| 154 | 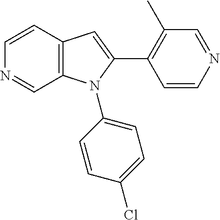 [SMILES: Cc1cnccc1-c1cc2ccncc2n1-c1ccc(Cl)cc1] | 4-[1-(4-Chlorophenyl)-1H- pyrrolo[2,3-c]pyridin-2-yl]- 3-methylpyridine | White solid Yield 29.5 mg, 8.45% HRMS (ESI+) calcd for [MH]+ of C19H14ClN3 320.0954 found 320.0958. HPLC: Rt 3.71 min, 100% purity. |

or a pharmaceutically acceptable salt, or N-oxide thereof.

WO2014/140592 discloses methods for the production of the above-mentioned compounds.

In addition to the surprising activity of the compounds of formula (I) at the SSAO receptor, it has been surprisingly found that the claimed compounds have surprisingly low activity at the hERG ion channel. The person skilled in the art, for example a medicinal chemist, understands that low hERG activity is an important property for a pharmaceutical drug compound. Without wishing to be bound by theory, it is believed that the —WVR³ group as defined in the claims is especially advantageous in relation to reduced hERG activity.

It is expected that compounds of the invention may be prepared in the form of hydrates, and solvates. Any reference herein, including the claims herein, to “compounds with which the invention is concerned” or “compounds of the invention” or “the present compounds”, and the like, includes reference to salts, hydrates, and solvates of such compounds. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Individual compounds of the invention may exist in an amorphous form and/or several polymorphic forms and may be obtained in different crystal habits. Any reference herein, including the claims herein, to “compounds with which the invention is concerned” or “compounds of the invention” or “the present compounds”, and the like, includes reference to the compounds irrespective of amorphous or polymorphic form.

Since compounds of the invention have a nitrogen atom in an aromatic ring they may form N-oxides, and the invention includes compounds of the invention in their N-oxide form.

### Definitions

The following definitions shall apply throughout the specification and the appended claims, unless otherwise stated or indicated.

The term “C₁₋₄-alkyl” denotes a straight or branched alkyl group having from 1 to 4 carbon atoms. For parts of the range C₁₋₄-alkyl all subgroups thereof are contemplated such as C₁₋₃-alkyl, C₁₋₂-alkyl, C₂₋₄-alkyl, C₂₋₃-alkyl and C₃₋₄-alkyl. Examples of said C₁₋₄-alkyl include methyl, ethyl, n-propyl, isopropyl, n-butyl, isobutyl, sec-butyl and tert-butyl.

Unless otherwise specified, the term “C₃₋₇-cycloalkyl” refers to a monocyclic saturated or partially unsaturated hydrocarbon ring system having from 3 to 7 carbon atoms. Examples of said C₃₋₇-cycloalkyl include cyclopropyl, cyclobutyl, cyclopentyl, cyclohexyl, cyclohexenyl, cycloheptyl, and cycloheptenyl. For parts of the range “C₃₋₇-cycloalkyl” all subgroups thereof are contemplated such as C₃₋₇-cycloalkyl, C₃₋₆-cycloalkyl, C₃₋₅-cycloalkyl, C₃₋₄-cycloalkyl, C₄₋₇-cycloalkyl, C₄₋₆-cycloalkyl, C₄₋₅-cycloalkyl, C₅₋₇-cycloalkyl, C₅₋₆-cycloalkyl, and C₆₋₇-cycloalkyl.

The term “C₁₋₄-alkoxy” refers to a straight or branched C₁₋₄-alkyl group which is attached to the remainder of the molecule through an oxygen atom. For parts of the range C₁₋₄-alkoxy, all subgroups thereof are contemplated such as C₁₋₃-alkoxy, C₁₋₂-alkoxy, C₂₋₄-alkoxy, C₂₋₃-alkoxy and C₃₋₄-alkoxy. Examples of said C₁₋₄-alkoxy include methoxy, ethoxy, n-propoxy, isopropoxy, n-butoxy, isobutoxy, sec-butoxy and tert-butoxy.

The term “haloC₁₋₄-alkoxy” refers to a straight or branched C₁₋₄-alkyl group which is attached to the remainder of the molecule through an oxygen atom and has one or more hydrogen atoms thereof replaced with halogen such as fluoro or chloro. For parts of the range C₁₋₄-alkoxy, all subgroups thereof are contemplated. Examples of said C₁₋₄-alkoxy include trifluoromethoxy.

The term “hydroxy-C₁₋₄-alkyl” denotes a straight or branched C₁₋₄-alkyl group that has one or more hydrogen atoms thereof replaced with OH. Examples of said hydroxy-C₁₋₄-alkyl include hydroxymethyl, 2-hydroxyethyl and 2,3-dihydroxypropyl.

The term “halo-C₁₋₄-alkyl” denotes a straight or branched C₁₋₄-alkyl group that has one or more hydrogen atoms thereof replaced with halogen. Examples of said halo-C₁₋₄-alkyl include fluoromethyl, trifluoromethyl, trichloromethyl and 2-fluoroethyl.

The term “cyano-C₁₋₄-alkyl” denotes a straight or branched C₁₋₄-alkyl group that has one or more hydrogen atoms thereof replaced with cyano. Examples of said cyano-C₁₋₄-alkyl include cyanomethyl, 2-cyanoethyl and 3-cyanopropyl.

The term “amino-C₁₋₄-alkyl” denotes a straight or branched C₁₋₄-alkyl group substituted with an amino group. Examples of said amino-C₁₋₄-alkyl group include aminomethyl and 2-aminoethyl.

The term “C₁₋₄-alkylamino-C₁₋₄-alkyl” denotes an amino-C₁₋₄-alkyl group as defined above, wherein the amino group is substituted with a straight or branched C₁₋₄-alkyl group.

Examples of said C₁₋₄-alkylamino-C₁₋₄-alkyl include methylaminoethyl and ethylaminopropyl.

The term “di(C₁₋₄-alkyl)amino-C₁₋₄-alkyl” denotes an amino-C₁₋₄-alkyl group as defined above, wherein the amino group is disubstituted with straight or branched C₁₋₄-alkyl groups, which can be the same or different. Examples of said di(C₁₋₄-alkyl)amino-C₁₋₄-alkyl include N,N-dimethylaminomethyl, N-ethyl-N-methylaminoethyl and N,N-diethylaminomethyl.

The terms “heteroaryl” and “heteroaromatic ring” denote a monocyclic heteroaromatic ring comprising 5 to 6 ring atoms in which one or more of the ring atoms are other than carbon, such as nitrogen, sulphur or oxygen. Examples of heteroaryl groups include furyl, pyrrolyl, thienyl, oxazolyl, isoxazolyl, imidazolyl, thiazolyl, isothiazolyl, pyridinyl, pyrimidinyl, tetrazolyl, pyrazolyl, pyridazinyl, pyrazinyl and thiadiazolyl.

The terms “heterocyclyl” and “heterocyclic ring” denote a non-aromatic, fully saturated or partially unsaturated, preferably fully saturated, monocyclic ring system having from 3 to 7 ring atoms, especially 5 or 6 ring atoms, in which one or more of the ring atoms are other than carbon, such as nitrogen, sulphur or oxygen. Examples of heterocyclic groups include piperidinyl, morpholinyl, homomorpholinyl, azepanyl, piperazinyl, oxo-piperazinyl, diazepinyl, tertahydropyridinyl, tetrahydropyranyl, pyrrolidinyl, tertrahydrofuranyl, and dihydropyrrolyl, groups.

The term “heterocyclic-C₁₋₄-alkyl” refers to a heterocyclic ring that is directly linked to a straight or branched C₁₋₄-alkyl group via a carbon or nitrogen atom of said ring. Examples of said heterocyclic-C₁₋₄-alkyl include piperidin-4-ylmethyl, piperidin-1-ylmethyl, morpholin-4-yl-methyl and piperazin-4-ylmethyl. The C₁₋₄-alkyl part, which includes methylene, ethylene, propylene or butylene, is optionally substituted by one or more substituents selected from halogen, amino, methoxy, or hydroxyl.

The term “C₁₋₄-alkylene” denotes a straight or branched divalent saturated hydrocarbon chain having from 1 to 4 carbon atoms. The C₁₋₄-alkylene chain may be attached to the rest of the molecule and to the radical group through one carbon within the chain or through any two carbons within the chain. Examples of C₁₋₄-alkylene radicals include methylene [—CH₂—], 1,2-ethylene [—CH₂—CH₂—], 1,1-ethylene [—CH(CH₃)—], 1,2-propylene [—CH₂—CH(CH₃)—] and 1,3-propylene [—CH₂—CH₂—CH₂—]. When referring to a “C₁₋₄-alkylene” radical, all subgroups thereof are contemplated, such as C₁₋₂-alkylene, C₂₋₃-alkylene, or C₃₋₄-alkylene.

“Halogen” refers to fluorine, chlorine, bromine or iodine, preferably fluorine and chlorine, most preferably fluorine.

“Hydroxy” refers to the —OH radical.

“Cyano” refers to the —CN radical.

“Oxo” refers to the carbonyl group ═O.

“Optional” or “optionally” means that the subsequently described event or circumstance may but need not occur, and that the description includes instances where the event or circumstance occurs and instances in which it does not.

Throughout the specification and the appended claims, a given chemical formula or name shall also encompass all salts, hydrates, solvates, and N-oxide forms thereof. Further, a given chemical formula or name shall encompass all tautomeric and stereoisomeric forms thereof. Tautomers include enol and keto forms. Stereoisomers include enantiomers and diastereomers. Enantiomers can be present in their pure forms, or as racemic (equal) or unequal mixtures of two enantiomers. Diastereomers can be present in their pure forms, or as mixtures of diastereomers. Diastereomers also include geometrical isomers, which can be present in their pure cis or trans forms or as mixtures of those.

The compounds of the formulae disclosed herein may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

### The Group Y

In an embodiment Y is from hydrogen, hydroxyl, —NH₂, —NH—C₁₋₄-alkyl such as —NH-Methyl, —NH-ethyl, or —NH-isopropyl, —NH-halo-C₁₋₄-alkyl such as —NHtrifluoromethyl, or —C₁₋₄-alkoxy such as methoxy. In an embodiment Y is hydrogen.

### The Group Z

In an embodiment Z is hydrogen, halogen such as fluoro or chloro, hydroxyl, cyano, C₁₋₄-alkyl such as methyl or isopropyl, halo-C₁₋₄-alkyl such as triflouromethyl, C₁₋₄-alkoxy such as methoxy, halo-C₁₋₄-alkoxy such as trifluoromethoxy, —CONH₂, —SO₂NH₂, —NH₂, —NHC₁₋₄-alkyl such as —NH-Methyl, —NH-ethyl, or —NH-isopropyl, or —NHhalo-C₁₋₄-alkyl. In an embodiment Z is hydrogen.

### The Group R¹

In an R¹ embodiment is a phenyl ring, or a 5 or 6-membered heteroaryl ring either ring being optionally substituted with one or more substituents selected from halogen such as fluoro or chloro, cyano, C₁₋₄-alkyl such as methyl or isopropyl, halo-C₁₋₄-alkyl such as trifluoromethyl, cyano-C₁₋₄-alkyl such as methylcyano, —OR⁵ such as methoxy or trifluoromethoxy, —NR^{4A}R^{4B} such as —NH₂, —NHMethyl, —NHisopropyl, —NR⁶C(O)OR⁵, —NR⁶C(O)R⁵, —NR⁶C(O)NR^{4A}R^{4B}, —C(O)NR^{4A}R^{4B}, —C(O)R⁵ such as —COCH₃, —C(O)OR⁵, and —NR⁶S(O)₂R⁵. In an embodiment R¹ is optionally substituted phenyl, pyridyl, pyrrole, furan, imidazole, or thiophene.

In an embodiment R¹ is a phenyl ring, or a 5 or 6-membered heteroaryl ring substituted with a 3-7 membered cycloalkyl group such as cyclopropyl, cyclobutyl, cyclopentyl, cyclohexyl; preferably cyclopropyl.

R^{4A}, R^{4B} R⁵ and R⁶ are each independently selected from hydrogen, C₁₋₄-alkyl such as methyl, ethyl or isopropyl, or halo-C₁₋₄-alkyl such as trifluoromethyl, or

R^{4A} and R^{4B} together with the nitrogen to which they are attached form a 3-7 membered cyclic amino group such as aziridine, azetidine, oxetane, pyrrolidine, piperidine, piperazine, homopiperidine, homopiperazine, morpholine, or tetrahydrofuran, optionally substituted by one or more substituents selected from: halogen such as fluoro or chloro, hydroxyl, cyano, C₁₋₄-alkyl such as methyl or isopropyl, halo-C₁₋₄-alkyl such as triflouromethyl, C₁₋₄-alkoxy such as methoxy, halo-C₁₋₄-alkoxy such as trifluoromethoxy, —CONH₂, —SO₂NH₂, —NH₂, —NHC₁₋₄-alkyl, —NHhalo-C₁₋₄-alkyl;

### The Group X

In an embodiment X is selected from —N═ or —C(R²)

### The Group R²

In an embodiment R² is hydrogen, halogen such as fluoro or chloro, cyano, C₁₋₄-alkyl such as methyl or ethyl or isopropyl, halo-C₁₋₄-alkyl such as trifluoromethyl. In an embodiment R² is hydrogen.

### The Group W

In an embodiment W is a phenyl ring. In an alternative embodiment W a 6-membered heterocyclic ring selected from pyridine, pyridazine, pyrazine, or pyrimidine. In an alternative embodiment W is a 5-membered ring selected from oxazole, thiazole or imidazole. In an embodiment W is imidazolyl and the imidazolyl ring is connected to the pyrrolopyridine core (i.e. the rest of the molecule) via an imidazolyl ring carbon atom. In an embodiment W is a pyrazole ring.

Any of the aforementioned rings are optionally substituted with one or more substituents as defined in claim 1. In an embodiment W is substituted with one or more groups selected from fluoro, chloro, cyano, methyl or trifluoromethyl.

In an embodiment W is a divalent group selected from any one of the following rings, any of which rings is optionally substituted with one or more substituents as defined in relation to formula (I).

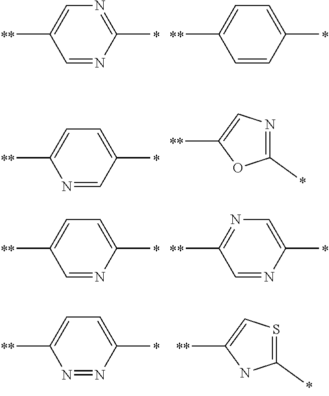

wherein the bond marked ** is directly connected to the rest of the molecule and the atom marked * is directly connected to V.

### The Group V

In an embodiment V is selected from a bond, —O—, —N(R⁶)— such as —NH— or —N(CH₃)—, —(C═O)—, —CONR⁶— such as —CONH— or —CON(CH₃)—, —NR⁶C(O)— such as —NHC(O)— or —N(CH₃)C(O)—, or —C₁₋₄-alkylene-, wherein the C₁₋₄-alkylene group is optionally substituted by halogen such as fluoro or chloro, and wherein any one of the carbon atoms of the C₁₋₄-alkylene group may be replaced by —O— or —N(R⁶)— such as —CH₂O—in either direction or —CH₂—NH—; —CH₂—N(CH₃)— in either direction.

### The Group R³

In an embodiment R³ is hydrogen. In an alternative embodiment R³ an optionally substituted 3-7 membered heterocyclic ring such as aziridine, azetidine, oxetane, pyrrolidine, piperidine, piperazine, homopiperidine, homopiperazine, morpholine, or tetrahydrofuran. In an embodiment R³ is an optionally substituted 3-7 membered cycloalkyl ring such as cyclopropyl, cyclobutyl, cyclopentyl or cyclohexyl. In an alternative embodiment R³ is an optionally substituted 5 or 6-membered heteroaryl ring such as imidazole, phenyl, pyridine, thophene. The optional substituents are defined in formula (I). In an embodiment any one of the rings is optionally substituted with one or more substituents selected from halogen such as fluoro or chloro, oxo, hydroxyl, cyano, C₁₋₄-alkyl such as methyl, ethyl, propyl, t-butyl, or isopropyl, halo-C₁₋₄-alkyl such as trifluoromethyl, cyano-C₁₋₄-alkyl, —OR⁵ such as methocy or trifluoromethoxy, —NR^{4A}R^{4B} such as —NH₂, NHmethyl, or morpholine or piperidine, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵.

In an embodiment R³ is selected from the following ring systems:

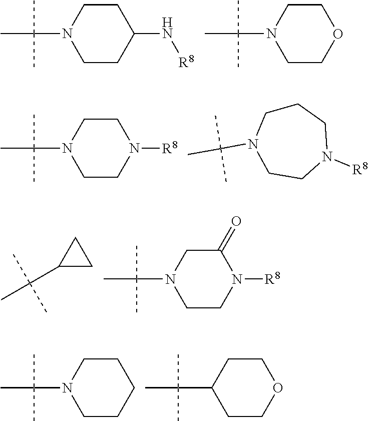

Wherein R⁸ is selected from hydrogen, CH₃, —CONH₂, —NHCONH₂, —S(O)₂CH₃, —COCH₃.

In an embodiment R³ is selected from the following ring systems:

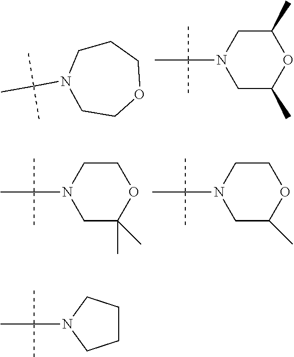

In an embodiment R³ is selected from hydrogen, —C₁₋₄-alkyl such as methyl, ethyl, propyl and isopropyl, and —C₁₋₄-alkyl-C₁₋₄-alkoxy such as —(CH₂)₂OCH₃.

In an embodiment the group —VR³ is selected from:

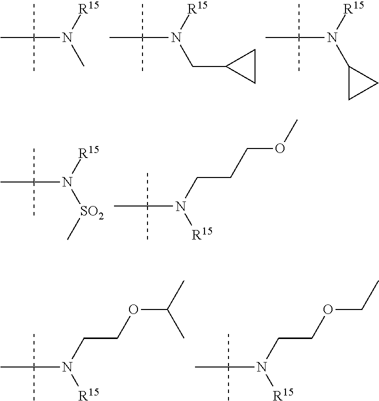

wherein R¹⁵ is hydrogen or methyl.

In an embodiment, the invention includes a compound of formula (Xa)

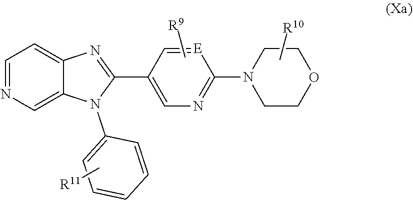

wherein E is —C═ or —N═,

R⁹ and R¹⁰ are each independently one or more substituents selected from hydrogen, halogen, cyano, oxo, C₁₋₄-alkyl such as methyl, —OC₁₋₄-alkyl such as OCH₃, and halo-C₁₋₄-alkyl; and

R¹¹ is one or more substituents selected from hydrogen, halogen such as fluoro and/or chloro, cyano, cyclopropyl, C₁₋₄-alkyl such as methyl, and halo-C₁₋₄-alkyl.

In a third aspect of the invention, the VAP-1 inhibitor is a compound of formula (II)

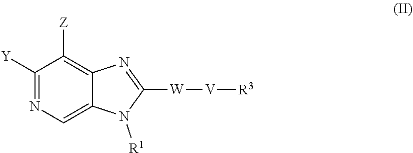

wherein:

Y is selected from hydrogen, hydroxyl, —NH₂, —NH—C₁₋₄-alkyl, —NH-halo-C₁₋₄-alkyl, or —C₁₋₄-alkoxy;

Z is selected from hydrogen, halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₂, —NHC₁₋₄-alkyl, or —NHhalo-C₁₋₄-alkyl;

R¹ is a phenyl ring, or a 5 or 6-membered heteroaryl ring, either ring optionally substituted with one or more substituents selected from halogen, cyano, C₁₋₄-alkyl, halo-C₁₋₄-alkyl, cyano-C₁₋₄-alkyl, —OR⁵, NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, and —NR⁶S(O)₂R⁵; wherein

R^{4A}, R^{4B} R⁵ and R⁶ are each independently selected from hydrogen, C₁₋₄-alkyl or halo-C₁₋₄-alkyl, or

R^{4A} and R^{4B} together with the nitrogen to which they are attached form a 3-7 membered cyclic amino group, optionally substituted by one or more substituents selected from: halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₂, —NHC₁₋₄-alkyl, —NHhalo-C₁₋₄-alkyl;

R^{7A} and R^{7B} are independently hydrogen, C₁₋₄-alkyl or halo-C₁₋₄-alkyl; and wherein

the group —WVR³ is selected from any one of groups (i)-(iv):

(i) W is a [6,5], [5,6], or [6,6] heteroaryl ring system comprising a phenyl ring or a 6-membered heteroaryl ring fused to a 5 or 6-membered heteroaryl or heterocyclic ring, the fused ring system being optionally substituted on either or both rings with one or more groups selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵, and

V is a direct bond, and

R³ is hydrogen;

(ii) W is a phenyl ring or a 5 or 6-membered heteroaryl ring, either ring optionally substituted with one or more groups selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵, and

V is —NR⁶—, and

R³ is a C₁₋₆-alkyl group substituted with one or more substituents selected from the group consisting of: halogen, hydroxyl, cyano, oxo, and NR^{7A}R^{7B};

(iii) W is a 5 or 6-membered heterocyclic ring optionally substituted with one or more substituents selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵,

V is a direct bond, and

R³ is a phenyl ring or a 5 or 6-membered heteroaryl ring optionally substituted with one or more substituents selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵;

(iv) W is a direct bond, V is a group selected from **—(C═O)—(CH₂)ₙ—, —CONR⁶—(CH₂)ₙ—, **—NR⁶C(O)—(CH₂)ₙ—, **—NR⁶C(O)O—(CH₂)ₙ— wherein the bond marked ** is connected to the rest of the molecule, or —C₁₋₄-alkylene-, wherein any one of the the —(CH₂)— groups, including the C₁₋₄-alkylene group, group is optionally substituted by halogen, and wherein any one of the carbon atoms of the C₁₋₄-alkylene group may be replaced by —O— or —N(R⁶)—, and

n is 0, 1, 2, 3, or 4

R³ is selected from:

a C₁₋₆-alkyl group optionally substituted with one or more substituents selected from the group consisting of: halogen, hydroxyl, cyano, oxo, C₁₋₄ alkoxy, C₁₋₄alkoxy and NR^{7A}R^{7B}; or a 3-7 membered heterocyclic or cycloalkyl ring, a phenyl ring, or a 5 or 6-membered heteroaryl ring, any of which rings is optionally substituted with a group selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵.

In a feature of the third aspect of the invention, W may be a [6,5] heteroaryl ring system formed by fusing together phenyl and pyrrolidinyl or imidazolyl and wherein either ring is optionally substituted as set out above for the third aspect of the invention, preferably wherein W has the formula A1 or A2:

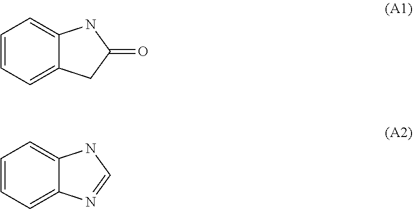

wherein W is optionally substituted on either ring as set out above for the third aspect of the invention, and wherein W is directly connected to the rest of the molecule via a carbon atom on the phenyl ring;

(ii) —WVR³ may be as defined in group (ii), and R³ may be C₁₋₆-alkyl substituted with one or more groups selected from fluoro, chloro, hydroxyl and C₁₋₄alkyl;

(iii) —WVR³ is as defined in group (ii), and R³ may be —CH₂C(CH₃)₂OH;

(iv) —WVR³ is as defined in group (iii), and W may be a ring selected from piperidine, morpholine, pyrrolidine, and piperazine, any of which is optionally substituted as set out above for the third aspect of the invention, preferably wherein —WVR³ is

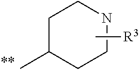

wherein the bond marked ** is directly connected to the rest of the molecule; or

(v) —WVR³ is as defined in group (iv), wherein V is selected from any one of —CONR⁶—, —CONR⁶—(CH₂)—, NR⁶C(O)—, —NR⁶C(O)—(CH₂)—, —NR⁶C(O)O—, —NR⁶C(O)O—(CH₂)—, —(CH₂)—, —(CH₂)₂—, and —(CH₂)₃—, and/or wherein R³ is a group selected from phenyl, imidazolyl, tetrahydropyranyl, piperidinyl, and piperazinyl, and one of which rings is optionally substituted according to the third aspect of the invention.

In a further feature of the third aspect of the invention,

(A) Y is hydrogen;

(B) Z is hydrogen; and/or

(C) R⁶ is hydrogen.

Specific exemplary VAP-1 inhibitors of the third aspect of the innovation include those disclosed in WO2016/042332, which is incorporated herein by reference. Those VAP-1 inhibitors include

### Example 155—5-[3-(4-Fluorophenyl)-3H-imidazo[4,5-c]pyridin-2-yl]-1H-1,3-benzodiazole

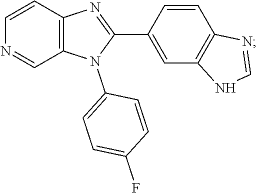 [SMILES: Fc1ccc(-n2c(-c3ccc4nc[nH]c4c3)nc3ccncc32)cc1]

### Example 156—1-({5-[3-(4-Fluorophenyl)-3H-imidazo[4,5-c]pyridin-2-yl]pyrimidin-2-yl}amino)-2-methylpropan-2-ol

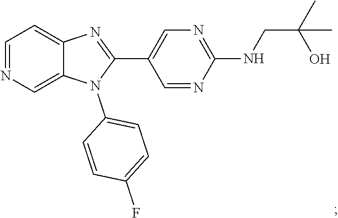 [SMILES: CC(C)(O)CNc1ncc(-c2nc3ccncc3n2-c2ccc(F)cc2)cn1]

### Example 157—2-Methyl-1-({5-[3-(4-methylphenyl)-3H-imidazo[4,5-c]pyridin-2-yl]pyrimidin-2-yl}amino)propan-2-ol

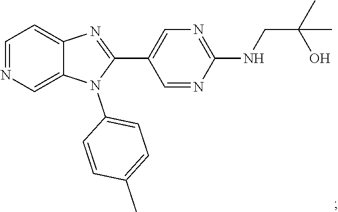 [SMILES: Cc1ccc(-n2c(-c3cnc(NCC(C)(C)O)nc3)nc3ccncc32)cc1]

### Example 158—4-{4-[3-(4-Chlorophenyl)-3H-imidazo[4,5-c]pyridin-2-yl]piperidin-1-yl}pyridine; bis(formic acid)

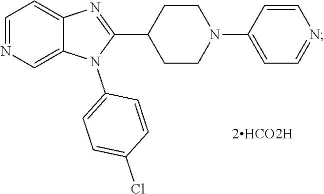 [SMILES: Clc1ccc(-n2c(C3CCN(c4ccncc4)CC3)nc3ccncc32)cc1]

### Example 159—6-{4-[3-(4-Chlorophenyl)-3H-imidazo[4,5-c]pyridin-2-yl]piperidin-1-yl}-3,4-dihydropyrimidin-4-one

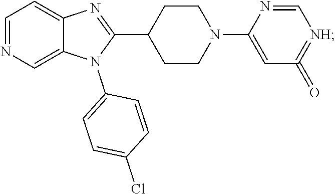

### Example 160—3-{[3-(4-Chlorophenyl)-3H-imidazo[4,5-c]pyridin-2-yl]methyl}pyridine

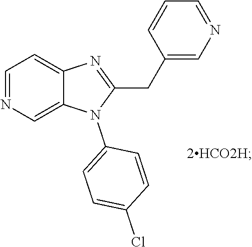 [SMILES: Clc1ccc(-n2c(Cc3cccnc3)nc3ccncc32)cc1]

### Example 161—1-{3-[3-(4-chlorophenyl)-3H-imidazo[4,5-c]pyridin-2-yl]propyl}-1H-imidazole

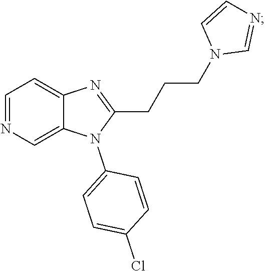 [SMILES: Clc1ccc(-n2c(CCCn3ccnc3)nc3ccncc32)cc1]

### Example 162—3-(4-Fluorophenyl)-N-(oxan-4-ylmethyl)-3H-imidazo[4,5-c]pyridine-2-carboxamide

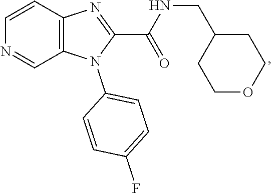 [SMILES: O=C(NCC1CCOCC1)c1nc2ccncc2n1-c1ccc(F)cc1]

or a pharmaceutically acceptable salt, or N-oxide thereof.

WO2016/042332 discloses methods for the production of the above-mentioned compounds.

Further specific exemplary VAP-1 inhibitors of the third aspect of the innovation include those disclosed in WO2016/042331, which is incorporated herein by reference. Those VAP-1 inhibitors include

| | | | Intermediate(s) used, |
|---|---|---|---|
| Ex | Structure | Name | Form, Yield, LCMS, HPLC |
| 163 | 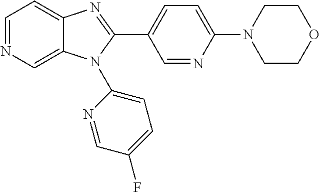 [SMILES: Fc1ccc(-n2c(-c3ccc(N4CCOCC4)nc3)nc3ccncc32)nc1] | 4-{5-[3-(5-Fluoropyridin-2- yl)-3H-imidazo[4,5- c]pyridin-2-yl]pyridin-2- yl}morpholine | pink solid Yield 25.0 mg, 4.17% HRMS (ES⁺) calculated for [M + H] of C₂₀H₁₇FN₆O: 377.1526, found 377.1524. HPLC: Rt: 4.13 min, 98.4% purity. |
| 164 | 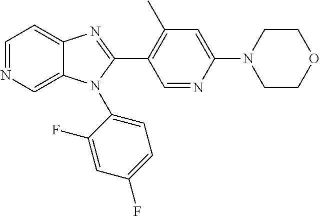 [SMILES: Cc1cc(N2CCOCC2)ncc1-c1nc2ccncc2n1-c1ccc(F)cc1F] | 4-{5-[3-(2,4- Difluorophenyl)-3H- imidazo[4,5-c]pyridin-2-yl]- 4-methylpyridin-2- yl}morpholine | Yellow solid Yield 17.0 mg, 2.64% HRMS (ES⁺) calculated for [M + H] of C₂₂H₁₉F₂N₅O: 408.1636, found 408.1635. HPLC: Rt 4.25 min, 99.4% purity |
| 165 | 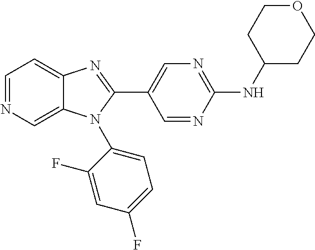 [SMILES: Fc1ccc(-n2c(-c3cnc(NC4CCOCC4)nc3)nc3ccncc32)c(F)c1] | 5-[3-(2,4-Difluorophenyl)- 3H-imidazo[4,5-c]pyridin-2- yl]-N-(oxan-4-yl)pyrimidin- 2-amine | White solid Yield 44.2 mg, 9.58% HRMS (ES⁺) calculated for [M + H] of C₂₁H₁₈F₂N₆O: 409.1588, found 409.1587. UPLC: Rt 1.99 min, 99.2% purity |
| 166 | 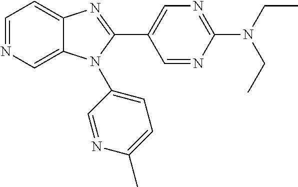 [SMILES: CCN(CC)c1ncc(-c2nc3ccncc3n2-c2ccc(C)nc2)cn1] | N,N-Diethyl-5-[3-(6- methylpyridin-3-yl)-3H- imidazo[4,5-c]pyridin-2- yl]pyrimidin-2-amine | White solid Yield 42.0 mg, 9.36% HRMS (ES⁺) calculated for [M + H] of C₂₀H₂₁N₇: 360.1937, found 360.1935. UPLC: Rt 2.12 min, 98.7% purity |
| 167 | 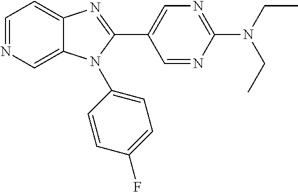 [SMILES: CCN(CC)c1ncc(-c2nc3ccncc3n2-c2ccc(F)cc2)cn1] | N,N-Diethyl-5-[3-(4- fluorophenyl)-3H- imidazo[4,5-c]pyridin-2- yl]pyrimidin-2-amine | Orange solid Yield 35.1 mg, 7.87% HRMS (ES⁺) calculated for [M + H] of C₂₀H₁₉FN₆: 363.1733, found 363.1737. UPLC: Rt 2.38 min, 98.7% purity |
| 168 | 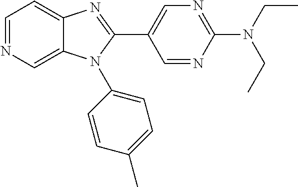 [SMILES: CCN(CC)c1ncc(-c2nc3ccncc3n2-c2ccc(C)cc2)cn1] | N,N-Diethyl-5-[3-(4- methylphenyl)-3H- imidazo[4,5-c]pyridin-2- yl]pyrimidin-2-amine | Off white solid Yield 27.0 mg, 6.00% HRMS (ES⁺) calculated for [M + H] of C₂₁H₂₂N₆: 359.1984, found 359.1973. UPLC: Rt 2.52 min, 99.3% purity |
| 169 | 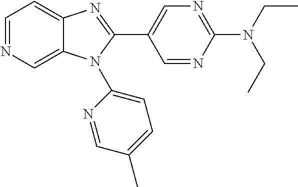 [SMILES: CCN(CC)c1ncc(-c2nc3ccncc3n2-c2ccc(C)cn2)cn1] | N,N-Diethyl-5-[3-(5- methylpyridin-2-yl)-3H- imidazo[4,5-c]pyridin-2- yl]pyrimidin-2-amine | White solid Yield 34.2 mg, 7.62% HRMS (ES⁺) calculated for [M + H] of C₂₀H₂₁N₇: 360.1937, found 360.1938. UPLC: Rt 2.31 min, 99.7% purity |
| 170 | 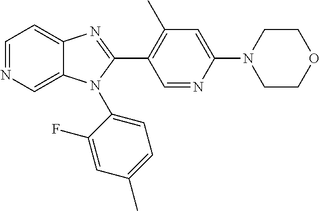 [SMILES: Cc1ccc(-n2c(-c3cnc(N4CCOCC4)cc3C)nc3ccncc32)c(F)c1] | 4-{5-[3-(2-Fluoro-4- methylphenyl)-3H- imidazo[4,5-c]pyridin-2-yl]- 4-methylpyridin-2- yl}morpholine | yellow solid Yield 22.0 mg, 9.1% HRMS (ES⁺) calculated for [M + H] of C₂₃H₂₂FN₅O: 404.1887, found 404.1888. HPLC: Rt 4.45 min, 99.3% purity. |
| 171 | 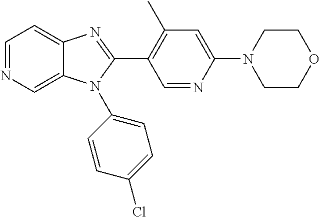 [SMILES: Cc1cc(N2CCOCC2)ncc1-c1nc2ccncc2n1-c1ccc(Cl)cc1] | 4-{5-[3-(4-Chlorophenyl)- 3H-imidazo[4,5-c]pyridin-2- yl]-4-methylpyridin-2- yl}morpholine | Off white solid Yield 7.20 mg, 1.30% HRMS (ES+) calculated for [M + H] of C22H20ClN5O: 406.1435, found 406.1433. UPLC: Rt 1.99 min, 100% purity |
| 172 | 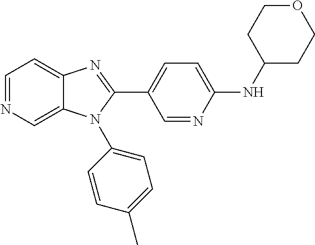 [SMILES: Cc1ccc(-n2c(-c3ccc(NC4CCOCC4)nc3)nc3ccncc32)cc1] | 5-[3-(4-Methylphenyl)-3H- imidazo[4,5-c]pyridin-2-yl]- N-(oxan-4-yl)pyridin-2- amine | Off white solid Yield 25.1 mg, 14.0% HRMS (ES+) calculated for [M + H] of C23H23N5O: 386.1981, found 386.1979. UPLC: Rt 1.83 min, 98.8% purity |
| 173 | 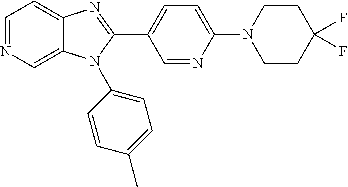 [SMILES: Cc1ccc(-n2c(-c3ccc(N4CCC(F)(F)CC4)nc3)nc3ccncc32)cc1] | 2-(4,4-Difluoropiperidin-1- yl)-5-[3-(4-methylphenyl)- 3H-imidazo[4,5-c]pyridin-2- yl]pyridine | White solid Yield 47.0 mg, 24.9% HRMS (ES+) calculated for [M + H] of C23H21F2N5: 406.1843, found 406.1842. UPLC: Rt 2.44 min, 98.6% purity |
| 174 | 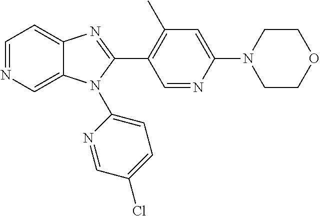 [SMILES: Cc1cc(N2CCOCC2)ncc1-c1nc2ccncc2n1-c1ccc(Cl)cn1] | 4-{5-[3-(5-Chloropyridin-2- yl)-3H-imidazo[4,5- c]pyridin-2-yl]-4- methylpyridin-2- yl}morpholine | off-white solid Yield 80.0 mg, 12.2% HRMS (ES⁺) calculated for [M + H] of C₂₁H₁₉ClN₆O: 407.1387, found 407.1385. UPLC: Rt: 1.90 min, 100% purity. |
| 175 | 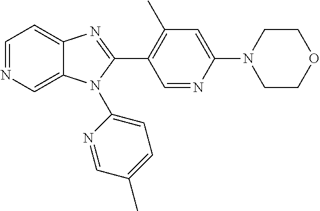 [SMILES: Cc1ccc(-n2c(-c3cnc(N4CCOCC4)cc3C)nc3ccncc32)nc1] | 4-{4-Methyl-5-[3-(5- methylpyridin-2-yl)-3H- imidazo[4,5-c]pyridin-2- yl]pyridin-2-yl}morpholine | White solid Yield 124 mg, 19.9% HRMS (ES+) calculated for [M + H] of C22H22N6O: 387.1933, found 387.1938. UPLC: Rt: 1.82 min, 99.0% purity |
| 176 | 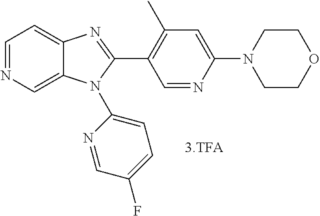 [SMILES: Cc1cc(N2CCOCC2)ncc1-c1nc2ccncc2n1-c1ccc(F)cn1] | 4-{5-[3-(5-Fluoropyridin-2- yl)-3H-imidazo[4,5- c]pyridin-2-yl]-4- methylpyridin-2- yl}morpholine; tris(trifluoroacetic acid) | Yellow solid Yield 18.0 mg, 2.12% HRMS (ES+) calculated for [M + H] of C21H19FN6O: 391.1682, found 391.1687. UPLC: Rt: 1.78 min, 97.3% purity |
| 177 | 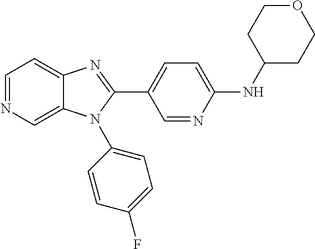 [SMILES: Fc1ccc(-n2c(-c3ccc(NC4CCOCC4)nc3)nc3ccncc32)cc1] | 5-[3-(4-Fluorophenyl)-3H- imidazo[4,5-c]pyridin-2-yl]- N-(oxan-4-yl)pyridin-2- amine | Off white solid Yield 40.0 mg, 22.3% HRMS (ES+) calculated for [M + H] of C22H20FN5O: 390.1730, found 390.1720. UPLC: Rt: 1.74 min, 97.7% purity |
| 178 | 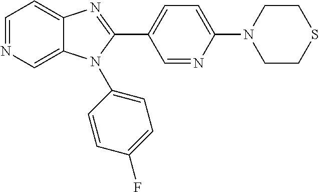 [SMILES: Fc1ccc(-n2c(-c3ccc(N4CCSCC4)nc3)nc3ccncc32)cc1] | 4-{5-[3-(4-Fluorophenyl)- 3H-imidazo[4,5-c]pyridin-2- yl]pyridin-2- yl}thiomorpholine | Off white solid Yield 49.0 mg, 27.2% HRMS (ES+) calculated for [M + H] of C21H18FN5S: 392.1345, found 392.1335. UPLC: Rt: 2.22 min, 99.2% purity |
| 179 | 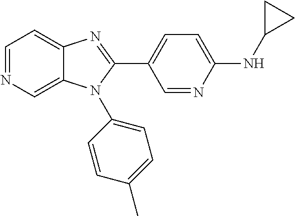 [SMILES: Cc1ccc(-n2c(-c3ccc(NC4CC4)nc3)nc3ccncc32)cc1] | N-Cyclopropyl-5-[3-(4- methylphenyl)-3H- imidazo[4,5-c]pyridin-2- yl]pyridin-2-amine | Off white solid Yield 24.1 mg, 15.2% HRMS (ES+) calculated for [M + H] of C21H19N5: 342.1718, found 342.1719. UPLC: Rt: 1.60 min, 98.7% purity |
| 180 | 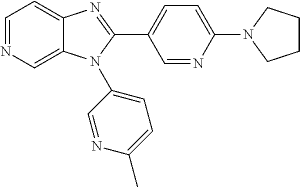 [SMILES: Cc1ccc(-n2c(-c3ccc(N4CCCC4)nc3)nc3ccncc32)cn1] | 5-[3-(6-Methylpyridin-3-yl)- 3H-imidazo[4,5-c]pyridin-2- yl]-2-(pyrrolidin-1- yl)pyridine | Off white solid Yield 49.2 mg, 29.8% HRMS (ES+) calculated for [M + H] of C21H20N6: 357.1828, found 357.1826. UPLC: Rt: 1.61 min, 99.7% purity |
| 181 | 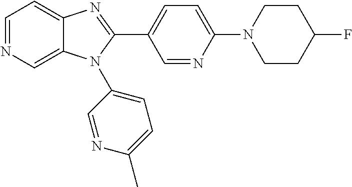 [SMILES: Cc1ccc(-n2c(-c3ccc(N4CCC(F)CC4)nc3)nc3ccncc32)cn1] | 2-(4-Fluoropiperidin-1-yl)-5- [3-(6-methylpyridin-3-yl)- 3H-imidazo[4,5-c]pyridin-2- yl]pyridine | Pale yellow solid Yield 64.1 mg, 35.6% HRMS (ES+) calculated for [M + H] of C22H21FN6: 389.1890, found 389.1886. UPLC: Rt: 1.91 min, 98.3% purity |
| 182 | 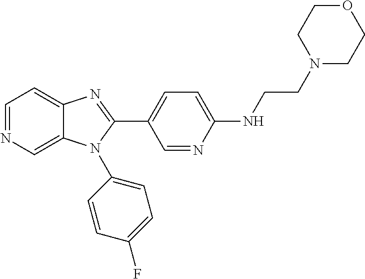 [SMILES: Fc1ccc(-n2c(-c3ccc(NCCN4CCOCC4)nc3)nc3ccncc32)cc1] | 5-[3-(4-Fluorophenyl)-3H- imidazo[4,5-c]pyridin-2-yl]- N-[2-(morpholin-4- yl)ethyl]pyridin-2-amine | Off white solid Yield 56.0 mg, 29.1% HRMS (ES+) calculated for [M + H] of C23H23FN6O: 419.1996, found 419.1984. UPLC: Rt: 1.64 min, 98.6% purity |
| 183 | 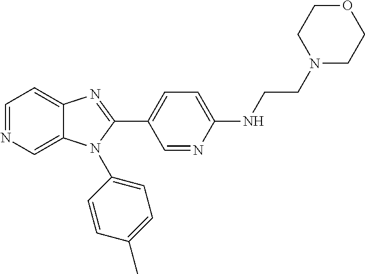 [SMILES: Cc1ccc(-n2c(-c3ccc(NCCN4CCOCC4)nc3)nc3ccncc32)cc1] | 5-[3-(4-Methylphenyl)-3H- imidazo[4,5-c]pyridin-2-yl]- N-[2-(morpholin-4- yl)ethyl]pyridin-2-amine | Off white solid Yield 35.1 mg, 18.2% HRMS (ES+) calculated for [M + H] of C24H26N6O: 415.2246, found 415.2245. UPLC: Rt: 1.73 min, 98.2% purity |
| 184 | 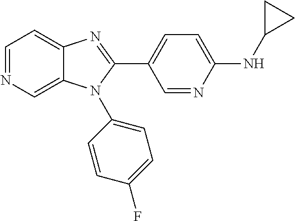 [SMILES: Fc1ccc(-n2c(-c3ccc(NC4CC4)nc3)nc3ccncc32)cc1] | N-Cyclopropyl-5-[3-(4- fluorophenyl)-3H- imidazo[4,5-c]pyridin-2- yl]pyridin-2-amine | Pale yellow solid Yield 75.5 mg, 35.6% HRMS (ES+) calculated for [M + H] of C20H16FN5: 346.1468, found 346.1470. UPLC: Rt: 1.75 min, 97.7% purity |
| 185 | 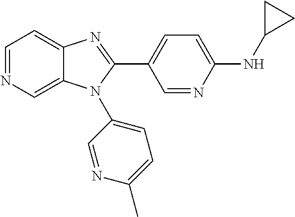 [SMILES: Cc1ccc(-n2c(-c3ccc(NC4CC4)nc3)nc3ccncc32)cn1] | N-Cyclopropyl-5-[3-(6- methylpyridin-3-yl)-3H- imidazo[4,5-c]pyridin-2- yl]pyridin-2-amine | White solid Yield 10.1 mg, 7.95% HRMS (ES+) calculated for [M + H] of C20H18N6: 343.1671, found 343.1675. UPLC: Rt: 1.54 min, 98.9% purity |
| 186 | 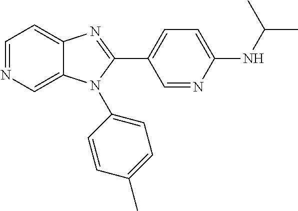 [SMILES: Cc1ccc(-n2c(-c3ccc(NC(C)C)nc3)nc3ccncc32)cc1] | 5-[3-(4-Methylphenyl)-3H- imidazo[4,5-c]pyridin-2-yl]- N-(propan-2-yl)pyridin-2- amine | Pale yellow solid Yield 28.0 mg, 13.1% HRMS (ES+) calculated for [M + H] of C21H21N5: 344.1875, found 344.1876. UPLC: Rt: 1.91 min, 100% purity |
| 187 | 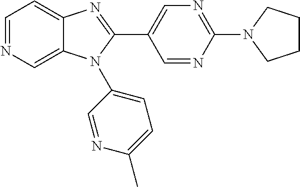 [SMILES: Cc1ccc(-n2c(-c3cnc(N4CCCC4)nc3)nc3ccncc32)cn1] | 5-[3-(6-Methylpyridin-3-yl)- 3H-imidazo[4,5-c]pyridin-2- yl]-2-(pyrrolidin-1- yl)pyrimidine | white solid Yield 127 mg, 28.5% HRMS (ES⁺) calculated for [M + H] of C₂₀H₁₉N₇: 358.1780, found 358.1779. UPLC: Rt 1.99 min, 99.2% purity |
| 188 | 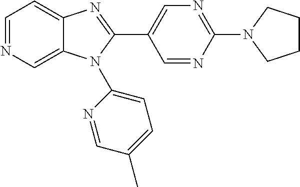 [SMILES: Cc1ccc(-n2c(-c3cnc(N4CCCC4)nc3)nc3ccncc32)nc1] | 5-[3-(5-Methylpyridin-2-yl)- 3H-imidazo[4,5-c]pyridin-2- yl]-2-(pyrrolidin-1- yl)pyrimidine | White solid Yield 48.2 mg, 13.5% HRMS (ES+) calculated for [M + H] of C20H19N7: 358.1780, found 358.1772. UPLC: Rt: 2.12 min, 99.2% purity |
| 189 | 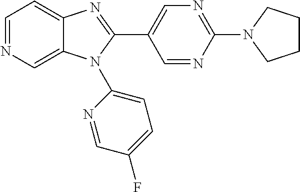 [SMILES: Fc1ccc(-n2c(-c3cnc(N4CCCC4)nc3)nc3ccncc32)nc1] | 5-[3-(5-Fluoropyridin-2-yl)- 3H-imidazo[4,5-c]pyridin-2- yl]-2-(pyrrolidin-1- yl)pyrimidine | White solid Yield 17.1 mg, 4.83% HRMS (ES+) calculated for [M + H] of C19H16FN7: 362.1529, found 362.1530. UPLC: Rt: 2.12 min, 98.8% purity |
| 190 | 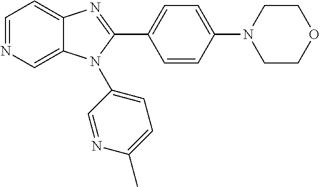 [SMILES: Cc1ccc(-n2c(-c3ccc(N4CCOCC4)cc3)nc3ccncc32)cn1] | 4-{4-[3-(6-Methylpyridin-3- yl)-3H-imidazo[4,5- c]pyridin-2- yl]phenyl}morpholine | White solid Yield 32.1 mg, 8.65% HRMS (ES+) calculated for [M + H] of C22H21N5O: 372.1824, found 372.1812. UPLC: Rt: 1.95 min, 99.1% purity |
| 191 | 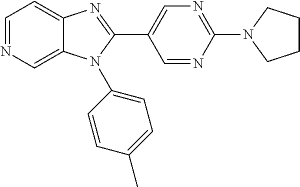 [SMILES: Cc1ccc(-n2c(-c3cnc(N4CCCC4)nc3)nc3ccncc32)cc1] | 5-[3-(4-Methylphenyl)-3H- imidazo[4,5-c]pyridin-2-yl]- 2-(pyrrolidin-1-yl)pyrimidine | White solid Yield 96.5 mg, 27.0% HRMS (ES+) calculated for [M + H] of C21H20N6: 357.1828, found 357.1828. UPLC: Rt: 2.32 min, 99.4% purity |
| 192 | 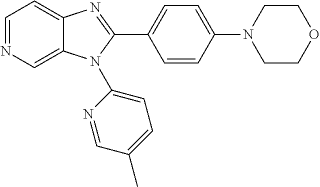 [SMILES: Cc1ccc(-n2c(-c3ccc(N4CCOCC4)cc3)nc3ccncc32)nc1] | 4-{4-[3-(5-Methylpyridin-2- yl)-3H-imidazo[4,5- c]pyridin-2- yl]phenyl}morpholine | White solid Yield 36.0 mg, 12.9% HRMS (ES+) calculated for [M + H] of C22H21N5O: 372.1824, found 372.1818. UPLC: Rt: 2.13 min, 100% purity |
| 193 | 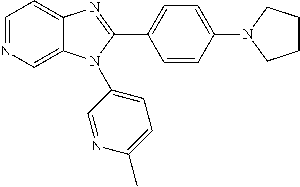 [SMILES: Cc1ccc(-n2c(-c3ccc(N4CCCC4)cc3)nc3ccncc32)cn1] | 2-Methyl-5-{2-[4-(pyrrolidin- 1-yl)phenyl]-3H- imidazo[4,5-c]pyridin-3- yl}pyridine | beige solid Yield 18.1 mg, 9.30% HRMS (ES⁺) calculated for [M + H] of C₂₂H₂₁N₅: 356.1875, found 356.1877. UPLC: Rt 2.30 min, 98.7% purity |
| 194 | 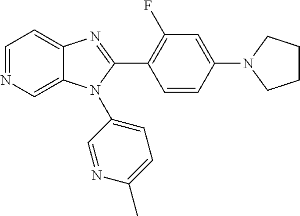 [SMILES: Cc1ccc(-n2c(-c3ccc(N4CCCC4)cc3F)nc3ccncc32)cn1] | 5-{2-[2-Fluoro-4-(pyrrolidin- 1-yl)phenyl]-3H- imidazo[4,5-c]pyridin-3-yl}- 2-methylpyridine | Yellow gum Yield 7.10 mg, 4.86% HRMS (ES+) calculated for [M + H] of C22H20FN5: 374.1781, found 374.1790. UPLC: Rt: 2.30 min, 98.1% purity |
| 195 | 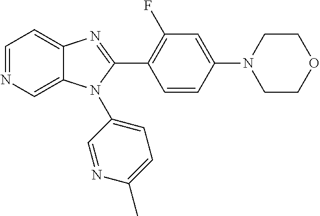 [SMILES: Cc1ccc(-n2c(-c3ccc(N4CCOCC4)cc3F)nc3ccncc32)cn1] | 4-{3-Fluoro-4-[3-(6- methylpyridin-3-yl)-3H- imidazo[4,5-c]pyridin-2- yl]phenyl}morpholine | White solid Yield 48.4 mg, 31.8% HRMS (ES+) calculated for [M + H] of C22H20FN5O: 390.1730, found 390.1725. UPLC: Rt: 1.98 min, 99.1% purity |
| 196 | 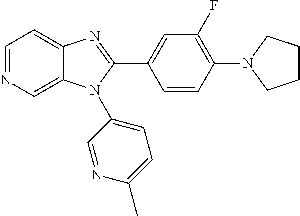 [SMILES: Cc1ccc(-n2c(-c3ccc(N4CCCC4)c(F)c3)nc3ccncc32)cn1] | 5-{2-[3-Fluoro-4-(pyrrolidin- 1-yl)phenyl]-3H- imidazo[4,5-c]pyridin-3-yl}- 2-methylpyridine | Yellow solid Yield 11.0 mg, 5.64% HRMS (ES+) calculated for [M + H] of C22H20FN5: 374.1781, found 374.1782. UPLC: Rt: 2.38 min, 99.1% purity |
| 197 | 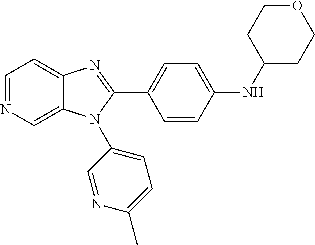 [SMILES: Cc1ccc(-n2c(-c3ccc(NC4CCOCC4)cc3)nc3ccncc32)cn1] | N-{4-[3-(6-Methylpyridin-3- yl)-3H-imidazo[4,5- c]pyridin-2-yl]phenyl}oxan- 4-amine | White solid Yield 55.2 mg, 26.2% HRMS (ES+) calculated for [M + H] of C23H23N5O: 386.1981, found 386.1987. UPLC: Rt: 1.97 min, 99.0% purity |
| 198 | 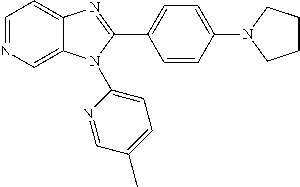 [SMILES: Cc1ccc(-n2c(-c3ccc(N4CCCC4)cc3)nc3ccncc32)nc1] | 5-Methyl-2-{2-[4-(pyrrolidin- 1-yl)phenyl]-3H- imidazo[4,5-c]pyridin-3- yl}pyridine | Off white solid Yield 50.2 mg, 41.3% HRMS (ES+) calculated for [M + H] of C22H21N5: 356.1875, found 356.1879. UPLC: Rt: 2.48 min, 98.7% purity |
| 199 | 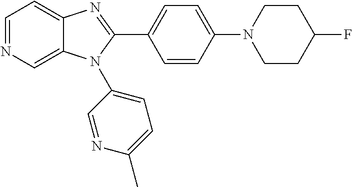 [SMILES: Cc1ccc(-n2c(-c3ccc(N4CCC(F)CC4)cc3)nc3ccncc32)cn1] | 5-{2-[4-(4-Fluoropiperidin-1- yl)phenyl]-3H-imidazo[4,5- c]pyridin-3-yl}-2- methylpyridine | White solid Yield 21.0 mg, 15.8% HRMS (ES+) calculated for [M + H] of C23H22FN5: 388.1937, found 388.1941. UPLC: Rt: 2.19 min, 100% purity |
| 200 | 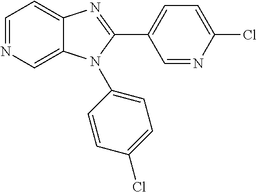 [SMILES: Clc1ccc(-n2c(-c3ccc(Cl)nc3)nc3ccncc32)cc1] | 2-Chloro-5-[3-(4- chlorophenyl)-3H- imidazo[4,5-c]pyridin-2- yl]pyridin | white solid Yield 55.6 mg, 36.0% HRMS (ES⁺) calculated for [M + H] of C₁₇H₁₀Cl₂N₄: 341.0361, found 341.0352. HPLC: Rt 5.13 min, 99.9% purity |
| 201 | 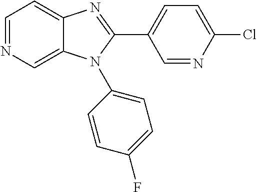 [SMILES: Fc1ccc(-n2c(-c3ccc(Cl)nc3)nc3ccncc32)cc1] | 2-Chloro-5-[3-(4- fluorophenyl)-3H- imidazo[4,5-c]pyridin-2- yl]pyridine | off white solid Yield 39.0 mg, 7.48% HRMS (ES⁺) calculated for [M + H] of C₁₇H₁₀ClFN₄: 325.0656, found 325.0642. HPLC: Rt: 4.76 min, 99.5%. |

or a pharmaceutically acceptable salt, or N-oxide thereof.

WO2016/042331 discloses methods for the production of the above-mentioned compounds.

### The Group Y

In an embodiment Y selected is from hydrogen, hydroxyl, —NH₂, —NH—C₁₋₄-alkyl such as —NH-Methyl, —NH-ethyl, or —NH-isopropyl, —NH-halo-C₁₋₄-alkyl such as —NHtrifluoromethyl, or —C₁₋₄-alkoxy such as methoxy. In an embodiment Y is hydrogen.

### The Group Z

In an embodiment Z is hydrogen, halogen such as fluoro or chloro, hydroxyl, cyano, C₁₋₄-alkyl such as methyl or isopropyl, halo-C₁₋₄-alkyl such as triflouromethyl, C₁₋₄-alkoxy such as methoxy, halo-C₁₋₄-alkoxy such as trifluoromethoxy, —CONH₂, —SO₂NH₂, —NH₂, —NHC₁₋₄-alkyl such as —NH-Methyl, —NH-ethyl, or —NH-isopropyl, or —NHhalo-C₁₋₄-alkyl. In an embodiment Z is hydrogen.

### The Group R¹

In an embodiment R¹ is a phenyl ring, or a 5 or 6-membered heteroaryl ring either ring being optionally substituted with one or more substituents selected from halogen such as fluoro or chloro, cyano, C₁₋₄-alkyl such as methyl or isopropyl, halo-C₁₋₄-alkyl such as trifluoromethyl, cyano-C₁₋₄-alkyl such as methylcyano, —OR⁵ such as methoxy or trifluoromethoxy, —NR^{4A}R^{4B} such as —NH₂, —NHMethyl, —NHisopropyl, —NR⁶C(O)OR⁵, —NR⁶C(O)R⁵, —NR⁶C(O)NR^{4A}R^{4B}, —C(O)NR^{4A}R^{4B}, —C(O)R⁵ such as —COCH₃, —C(O)OR⁵, and —NR⁶S(O)₂R⁵. In an embodiment R¹ is optionally substituted phenyl, pyridyl, pyrrole, furan, imidazole, or thiophene. In an embodiment R¹ is optionally substituted with one or more substituents selected from halogen and C₁₋₄alkyl, preferably the halogen is fluoro or chloro, and the C₁₋₄alkyl group is methyl.

In an embodiment R¹ is a phenyl ring, or a 5 or 6-membered heteroaryl ring substituted with a 3-7 membered cycloalkyl group such as cyclopropyl, cyclobutyl, cyclopentyl, cyclohexyl; preferably cyclopropyl.

R^{4A}, R^{4B} R⁵ and R⁶ are each independently selected from hydrogen, C₁₋₄-alkyl such as methyl, ethyl or isopropyl, or halo-C₁₋₄-alkyl such as trifluoromethyl, or

R^{4A} and R^{4B} together with the nitrogen to which they are attached form a 3-7 membered cyclic amino group such as aziridine, azetidine, oxetane, pyrrolidine, piperidine, piperazine, homopiperidine, homopiperazine, morpholine, or tetrahydrofuran, optionally substituted by one or more substituents selected from: halogen such as fluoro or chloro, hydroxyl, cyano, C₁₋₄-alkyl such as methyl or isopropyl, halo-C₁₋₄-alkyl such as trifluoromethyl, C₁₋₄-alkoxy such as methoxy, halo-C₁₋₄-alkoxy such as trifluoromethoxy, —CONH₂, —SO₂NH₂, —NH₂, —NHC₁₋₄-alkyl, —NHhalo-C₁₋₄-alkyl; R^{7A} and R^{7B} are independently hydrogen, C₁₋₄-alkyl such as methyl or isopropyl, or halo-C₁₋₄-alkyl such as trifluoromethyl.

The group —WVR³ is selected from any one of embodiments (i)-(iv), referred to as the first, second, third and fourth embodiments respectively:

(i) In a first embodiment, W is a [6,5], [5,6], or [6,6] heteroaryl ring system comprising a phenyl ring or a 6-membered heteroaryl ring such as pyridinyl, pyridazinyl, pyrazinyl, or pyrimidinyl fused to a 5 or 6-membered heteroaryl such as pyridinyl, pyridazinyl, pyrazinyl, pyrimidinyl, imidazolyy, oxazolyl, or thiazolyl or a heterocyclic ring such as pyrrolidinyl, the fused ring system being optionally substituted on either or both rings with one or more groups selected from halogen such as chloro and fluoro, oxo, hydroxyl, cyano, C₁₋₄-alkyl such as methyl, ethyl and isopropyl, halo-C₁₋₄-alkyl such as trifluoromethyl, cyano-C₁₋₄-alkyl such as cyanomethyl, —OR⁵ such as methoxy, —NR^{4A}R^{4B} such as —NH₂, NHMe, or —N(Me)₂, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵, and V is a direct bond, and R³ is hydrogen.

In an embodiment W is a [6,5] heteroaryl ring system, wherein the 6 membered ring is phenyl, and the 5-membered ring is pyrrolidinyl or imidazolyl and wherein the [6,5] ring system is connected to the rest of the molecule (i.e. the imidazopyridine core bearing Y, Z, and R¹) via the phenyl ring, and wherein either ring is optionally substituted as set out in claim 1. Preferred optional substituents on the W ring system are halogen, oxo and C₁₋₄-alkyl.

In an embodiment the group —WVR³ is A1 or A2 wherein the —WVR³ group is connected to the rest of the molecule via a phenyl ring carbon atom.

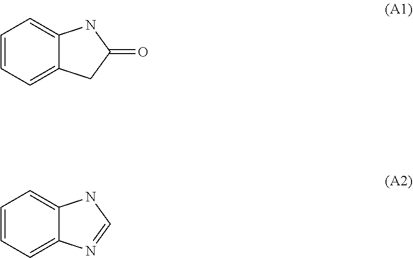 [SMILES: O=C1Cc2ccccc2N1 ; c1ccc2[nH]cnc2c1]

(ii) In a second embodiment W is a phenyl ring or a 5 or 6-membered heteroaryl ring such as pyridinyl, pyridazinyl, pyrazinyl, pyrimidinyl, imidazolyl, oxazolyl, or thiazolyl, either ring optionally substituted with one or more groups selected from halogen such as fluoro or chloro, oxo, hydroxyl, cyano, C₁₋₄-alkyl such as methyl, ethyl and isopropyl, halo-C₁₋₄-alkyl such as trifluoromethyl, cyano-C₁₋₄-alkyl such as cyanomethyl, —OR⁵ such as methoxy, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵, and

V is —NR⁶— such as —NH—, or —N(CH₃)—, and

R³ is a C₁₋₆-alkyl group such as methyl, ethyl, propyl, isopropyl, n-butyl, isobutyl, sec-butyl, or tert-butyl substituted with one or more substituents selected from the group consisting of: halogen, hydroxyl, cyano, oxo, and NR^{7A}R^{7B} such as —NH₂, —NHCH₃, —N(CH₃)₂.

Preferably R³ is substituted with one or more substituents selected from: hydroxyl, fluoro, chloro, and cyano.

In an embodiment W is a phenyl or 6 membered heteroaryl ring substituted in a 1,4 (i.e. para) pattern—in other words so that the atom to which the —VR³ group is connected is separated by two ring atoms from the atom to which the rest of the molecule is connected.

In an embodiment, W is a ring selected from phenyl, pyridinyl or pyrimidinyl. In an embodiment V is —NH— or —N(CH₃)—. In an embodiment R³ is —(CH₂)C(CH₃)₂OH.

In an embodiment W is a divalent group selected from any one of the following rings, any of which rings is optionally substituted as set out in claim 1

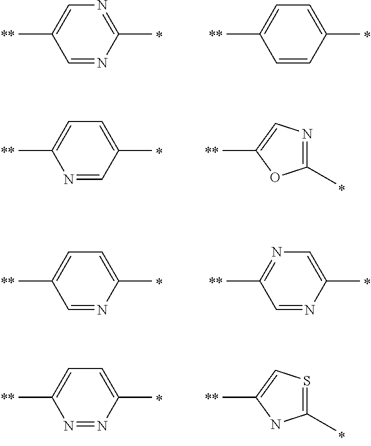

wherein the bond marked ** is directly connected to the rest of the molecule and the atom marked * is directly connected to V.

(iii) In the third embodiment W is a 5 or 6-membered heterocyclic ring such as piperidinyl, morpholinyl, or pyrrolidinyl optionally substituted with one or more substituents selected from halogen such as fluoro or chloro, oxo, hydroxyl, cyano, C₁₋₄-alkyl such as methyl, ethyl and isopropyl, halo-C₁₋₄-alkyl such as trifluoromethyl, cyano-C₁₋₄-alkyl such as cyanomethyl, —OR⁵ such as methoxy, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵,

V is a direct bond, and

R³ is a phenyl ring or a 5 or 6-membered heteroaryl ring such as pyridinyl, pyridazinyl, pyrazinyl, pyrimidinyl, imidazolyl, oxazolyl, or thiazolyl optionally substituted with one or more substituents selected from halogen such as fluoro or chloro, oxo, hydroxyl, cyano, C₁₋₄-alkyl such as methyl, ethyl and isopropyl, halo-C₁₋₄-alkyl such as trifluoromethyl, cyano-C₁₋₄-alkyl such as cyanomethyl, —OR⁵ such as methoxy, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵. In an embodiment W is a 6 membered heteroaryl ring substituted in a 1,4 pattern—in other words so that the atom to which the —R³ group is connected is separated by two ring atoms from the atom to which the rest of the molecule is connected. In an embodiment W is a piperidine ring.

In an embodiment the group —WVR³ is:

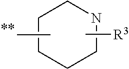

wherein the bond marked ** is directly connected to the rest of the molecule.

In an embodiment the group —WVR³ is:

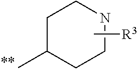

In an embodiment the group —WVR³ is:

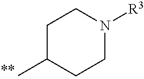

In an embodiment R³ is selected from phenyl, pyridyl and pyrimidinyl, any of which is optionally substituted with one or more groups selected from fluoro, chloro, oxo and C₁₋₄-alkyl. In an embodiment R³ is selected from phenyl, pyridyl and pyrimidinyl, any of which is optionally substituted with oxo.

(iv) In the fourth embodiment W is a direct bond, V is a group selected from **—(C═O)—(CH₂)ₙ— such as —C(O)—, —C(O)CH₂— or —C(O)(CH₂)₂—, **—CONR⁶—(CH₂)ₙ— such as —C(O)NR⁶—, —C(O)NR⁶CH₂— or —C(O)NR⁶(CH₂)₂—, **—NR⁶C(O)(CH₂)ₙ— such as —NR⁶C(O)—, —NR⁶C(O)CH₂— or —NR⁶C(O)(CH₂)₂—, or **—NR⁶C(O)O—(CH₂)ₙ— such as —NR⁶C(O)O—, —NR⁶C(O)OCH₂— or —NR⁶C(O)O(CH₂)₂— wherein the bond marked ** is connected to the rest of the molecule, or a C₁₋₄ alkylene group (i.e. —(CH₂)₁₋₄—) such as —(CH₂)—, —(CH₂)₂—, —(CH₂)₃—, or —(CH₂)₄— wherein one or more of the hydrogen atoms on any one of the aforementioned —(CH₂)— groups is optionally replaced by halogen such as fluoro, and wherein any one of the carbon atoms of the C₁₋₄ alkylene group may be replaced by —O— or —N(R⁶)—, and

n is 0, 1, 2, 3, or 4

R³ is selected from a C₁₋₆-alkyl group optionally substituted with one or more substituents selected from the group consisting of: halogen, hydroxyl, cyano, oxo, C₁₋₄ alkoxy, C₁₋₄haloalkoxy and NR^{7A}R^{7B}; or a 3-7 membered heterocyclic or cycloalkyl ring such as such as piperidinyl, pyrrolidinyl, morpholinyl, tetrahydropyranyl, cyclohexyl, cyclopentyl, or cyclopropyl, a phenyl ring, or a 5 or 6-membered heteroaryl ring such as pyridyl, pyridazinyl, pyrazinyl, pyrimidinyl, imidazolyl, oxazolyl, or thiazolyl, any of which rings is optionally substituted with a group selected from halogen such as fluoro or chloro, oxo, hydroxyl, cyano, C₁₋₄-alkyl such as methyl, ethyl and isopropyl, halo-C₁₋₄-alkyl such as trifluoromethyl, cyano-C₁₋₄-alkyl such as cyanomethyl, —OR⁵ such as methoxy, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵.

In an embodiment V is C₁₋₄ alkylene group optionally substituted with one or more fluoro, and R³ is phenyl, pyridyl or imidazolyl, any of which rings is optionally substituted as set out in claim 1.

In an embodiment V is —(C═O)—(CH₂)ₙ— or —CONR⁶—(CH₂)ₙ— and R³ is a 3-7 membered heterocyclic ring optionally substituted as set out in claim 1. In an embodiment R³ is tetrahydropyran.

In a fourth aspect of the invention, the VAP-1 inhibitor is a compound of Formula (III) or a pharmaceutically acceptable salt, or N-oxide thereof

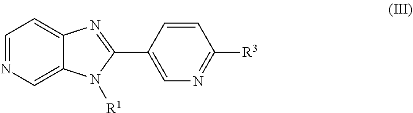

wherein

R¹ is a phenyl ring, or a 5 or 6-membered heteroaryl ring, either ring being optionally substituted with one or more substituents selected from halogen, cyano, C₁₋₄-alkyl, halo-C₁₋₄-alkyl, cyano-C₁₋₄-alkyl, a 3-7 membered cycloalkyl ring, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, and —NR⁶S(O)₂R⁵; wherein

R^{4A}, R^{4B} R⁵ and R⁶ are each independently selected from hydrogen, C₁₋₄-alkyl or halo-C₁₋₄-alkyl, or

R^{4A} and R^{4B} together with the nitrogen to which they are attached form a 3-7-membered cyclic amino group, optionally substituted by one or more substituents selected from: halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₂, —NHC₁₋₄-alkyl, —NHhalo-C₁₋₄-alkyl;

R³ is a 3-7 membered heterocyclic ring, a 3-7 membered cycloalkyl ring, or a 5 or 6-membered heteroaryl ring, any one of the rings being optionally substituted with one or more substituents selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵.

In a feature of the fourth aspect of the invention, R¹ is a phenyl ring optionally substituted with one or more substituents as defined for the fourth aspect of the invention.

In a further feature of the fourth aspect of the invention, the VAP-1 inhibitor is a compound of Formula (IIIa) or a pharmaceutically acceptable salt, or N-oxide thereof

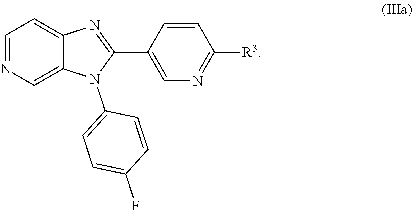

In another feature of the fourth aspect of the invention, R³ is a 3-7 membered heterocyclic ring optionally substituted with one or more substituents as defined for the fourth aspect of the invention. For instance, R³ may be a piperazine or morpholine ring optionally substituted with one or more substituents as defined for the fourth aspect of the invention. The piperazine or morpholine ring of R³ may be joined to the rest of the molecule through a nitrogen atom of that piperazine or morpholine ring. Wherein R³ is a piperazine ring, it may be substituted with at least one substituent as defined for the fourth aspect of the invention on a nitrogen atom in that piperazine ring.

Specific exemplary VAP-1 inhibitors of the fourth aspect of the innovation include those disclosed in WO2014/140592, which is incorporated herein by reference. Those VAP-1 inhibitors include

### 1-(4-{5-[3-(4-Fluorophenyl)-3H-imidazo[4,5-c]pyridin-2-yl]pyridin-2-yl}piperazin-1-yl)ethan-1-one (Compound 2)

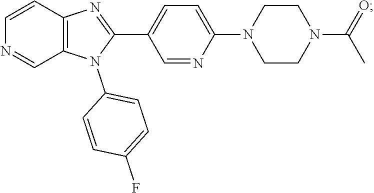 [SMILES: CC(=O)N1CCN(c2ccc(-c3nc4ccncc4n3-c3ccc(F)cc3)cn2)CC1]

### 4-{5-[3-(4-Fluorophenyl)-3H-imidazo[4,5-c]pyridin-2-yl]pyridin-2-yl}morpholine (Compound 4)

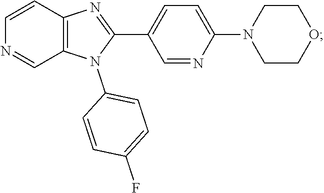 [SMILES: Fc1ccc(-n2c(-c3ccc(N4CCOCC4)nc3)nc3ccncc32)cc1]

and

### 1-{5-[3-(4-Fluorophenyl)-3H-imidazo[4,5-c]pyridin-2-yl]pyridin-2-yl}-4-methanesulfonylpiperazine (Compound 3)

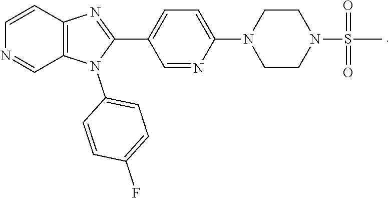 [SMILES: CS(=O)(=O)N1CCN(c2ccc(-c3nc4ccncc4n3-c3ccc(F)cc3)cn2)CC1]

Methods for the production of the above-mentioned compounds ios as described in WO2014/140592.

In a fifth aspect of the invention, the VAP-1 inhibitor is selected from the group consisting of (S)-carbidopa, benserazide, LJP1207, LJP1586, mofegiline, BTT1023, RTU-1096, PXS4728 and ASP8232 or a hydrate or pharmaceutically acceptable salt thereof.

Preferably the VAP-1 inhibitor is (S)-carbidopa.

(S)-Carbidopa has the Formula

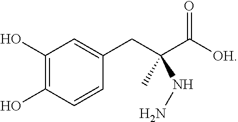 [SMILES: C[C@@](Cc1ccc(O)c(O)c1)(NN)C(=O)O]

Benserazide has the Formula

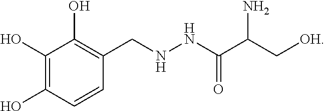 [SMILES: NC(CO)C(=O)NNCc1ccc(O)c(O)c1O]

LJP1207 has the Formula

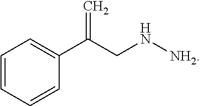

LJP1586 has the Formula

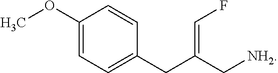

Mofegiline has the Formula

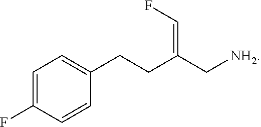 [SMILES: NC/C(=C/F)CCc1ccc(F)cc1]

BTT1023 has the Formula

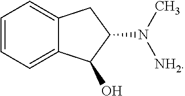

PXS4728 has the Formula

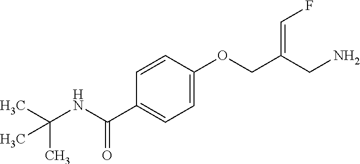 [SMILES: CC(C)(C)NC(=O)c1ccc(OC/C(=C/F)CN)cc1]

The peripheral decarboxylase inhibitors benserazide and (S)-carbidopa, often administered in combination with L-dopa in the treatment of Parkinson's disease, are also known to be very good inhibitors of VAP-1. Racemic Benserazide is preferred for use in the present invention. In an embodiment the Benserazide for use in the present invention is the (R)-enantiomer or the (S)-enantiomer.

Carbidopa exists as (R) and (S) enantiomers. Carbidopa is typically available as a mixture of the (R) and (S) enantiomers. Reference herein to “(S) carbidopa” includes any composition or mixture comprising (S) carbidopa, including for example substantially pure (S) carbidopa, or mixtures of (S) and (R) carbidopa, such as racemic mixtures. In an embodiment, the term “(S) carbidopa” as used herein means substantially pure (S) carbidopa.

VAP-1 Inhibitors for the Prevention and/or Treatment of Migraine

Migraine is an unpleasant condition which may interfere with a person's quality of life. Symptoms of migraine include pain (for instance, felt in the head, face, and/or neck), nausea, vomiting, increased sensitivity to light and sound, sweating, poor concentration, feeling very hot or very cold, abdominal pain, diarrhoea, and auras. Auras may describe visual problems (such as seeing flashing lights, zig-zag patterns or blind spots), numbness or a tingling sensation like pins and needles (this may starts in one hand and moves up a subject's arm before affecting the face, lips and tongue), feeling dizzy or off balance, difficulty speaking, and loss of consciousness. Some subjects may experience aura followed by only a mild headache or no headache at all.

An unmet medical need exists for new or improved prevention and/or treatments for migraine. Improved prevention and/or treatments may provide any or all of the following: superior symptom reduction (including pain relief); faster symptom relief (including pain relief); increased compliance; decreased likelihood of addiction; reduced treatment-related side effects; the ability to reduce exposure to other therapeutic agents that exhibit dose-dependent treatment-related side effects; or any other perceptible therapeutic benefit.

The applicants have discovered that compounds having VAP-1 inhibitory activity are surprisingly effective in the prevention and/or treatment of migraine. In vivo data in well-established models of migraine is provided herein. This data demonstrates the efficacy of a broad range of VAP-1 inhibitors in the prevention and/or treatment of migraine. Thus, the applicant demonstrates a credible link between the inhibition of VAP-1 activity and utility in the prevention and/or treatment migraine. It is therefore expected that substantially all VAP-1 inhibitors will be effective in the prevention and/or treatment of migraine. The following Examples of VAP-1 inhibitors having utility for the prevention and/or treatment of migraine are non-limiting, and should be considered as merely illustrative of the broad scope of the invention. Furthermore, it has been surprisingly found that the effect of a VAP-1 inhibitor, such as (S)-carbidopa, on migraine is independent of an effect (if any) on inflammation.

It has also been found that the VAP-1 inhibitor LJP1207 is surprisingly effective in the prevention and/or treatment of migraine.

It has also been found that the VAP-1 inhibitor (S)-carbidopa is surprisingly effective in the prevention and/or treatment of migraine.

It has also been found that the VAP-1 inhibitor 1-(4-{5-[3-(4-Fluorophenyl)-3H-imidazo[4,5-c]pyridin-2-yl]pyridin-2-yl}piperazin-1-yl)ethan-1-one (referred to as Compound 2) is surprisingly effective in the prevention and/or treatment of migraine.

It has also been found that the VAP-1 inhibitor 1-{5-[3-(4-Fluorophenyl)-3H-imidazo[4,5-c]pyridin-2-yl]pyridin-2-yl}-4-methanesulfonylpiperazine (referred to as Compound 3) is surprisingly effective in the prevention and/or treatment of migraine.

It has also been found that the VAP-1 inhibitor 4-{5-[3-(4-Fluorophenyl)-3H-imidazo[4,5-c]pyridin-2-yl]pyridin-2-yl}morpholine (referred to as Compound 4) is surprisingly effective in the prevention and/or treatment of migraine.

The present invention makes available a VAP-1 inhibitor for, or for use in the manufacture of a medicament for the prevention and/or treatment of migraine

The present invention makes available a method for the prevention and/or treatment of migraine, which comprises administering to a subject suffering from migraine an effective amount of a VAP-1 inhibitor.

The present invention makes available a pharmaceutical composition for use in the prevention and/or treatment of migraine, which comprises a VAP-1 inhibitor and a pharmaceutically acceptable carrier, excipient, or diluent.

Whilst it is understood that the VAP-1 inhibitors may prevention and treat migraine, the present invention may prevent migraine or may treat migraine.

The VAP-1 inhibitor may have the structure of any one of the specific Examples of VAP-1 inhibitor compounds. Preferably, the VAP-1 inhibitor is a compound selected from
1-(4-{5-[3-(4-Fluorophenyl)-3H-imidazo[4,5-c]pyridin-2-yl]pyridin-2-yl}piperazin-1-yl)ethan-1-one (compound 2);
4-{5-[3-(4-Fluorophenyl)-3H-imidazo[4,5-c]pyridin-2-yl]pyridin-2-yl}morpholine (compound 4); and
1-{5-[3-(4-Fluorophenyl)-3H-imidazo[4,5-c]pyridin-2-yl]pyridin-2-yl}-4-methanesulfonylpiperazine (compound 3)
   or an N-oxide, or a hydrate or a pharmaceutically acceptable salt thereof

A typical dosage of the compounds disclosed herein in the prevention and/or treatment of migraine may be in total daily dosage for a human of 1 to 2000 mg/day, preferably from 20 to 1000 mg/day, more preferably from 50 to 200 mg/day, most preferably from 50 to 150 mg/day.

In an embodiment, the compounds are dosed three times per day. For instance, 1-(4-{5-[3-(4-Fluorophenyl)-3H-imidazo[4,5-c]pyridin-2-yl]pyridin-2-yl}piperazin-1-yl)ethan-1-one (compound 2) may be administered at from 50 to 150 mg/day, and 1-{5-[3-(4-Fluorophenyl)-3H-imidazo[4,5-c]pyridin-2-yl]pyridin-2-yl}-4-methanesulfonylpiperazine (compound 3) and 4-{5-[3-(4-Fluorophenyl)-3H-imidazo[4,5-c]pyridin-2-yl]pyridin-2-yl}morpholine (compound 4) may be administered at from 10 to 50 mg/day.

The compounds may be administered in a variety of dosage forms. Thus, they can be administered orally, for example as a tablet, a capsule, a troche, a lozenge, an aqueous or oily suspension, a dispersible powder or granule. The compounds are preferably administered via the oral route. It will be understood, however, that the specific dose level for any particular patient will depend upon a variety of factors including the age, body weight, general health, sex, diet, time of administration, drug combination and the severity of the particular condition undergoing therapy.

### Compositions

A pharmaceutical composition containing the active ingredient, or active ingredients in the case of a combined preparation, may be in any suitable form, for example aqueous or non-aqueous solutions or suspensions, dispersible powders or granules, transdermal or transmucosal patches, creams, ointments or emulsions.

The pharmaceutical composition may be in the form of a sterile injectable aqueous or non-aqueous (e.g. oleaginous) solution or suspension. The sterile injectable preparation may also be in a sterile injectable solution or suspension in a non-toxic parenterally-acceptable diluent or solvent, for example as a solution in 1,3-butanediol. Among the acceptable vehicles and solvents that may be employed are water, phosphate buffer solution, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables. Suspensions may be formulated according to the known art using those suitable dispersing or wetting agents and suspending agents.

Aqueous suspensions contain the active ingredient, or active ingredients in the case of a combined preparation, in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents such as a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Non-aqueous (i.e. oily) suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are known.

The active agent may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical delivery, transdermal and transmucosal patches, creams, ointments, jellies, solutions or suspensions may be employed. For sub-lingual delivery, fast dissolving tablet formulations may be used, as well as a number of the presentations described above. For oral administration, the drug may be administered as tablets, capsules or liquids.

Formulations may conveniently be presented in unit dosage form, e.g., tablets and sustained release capsules, and in liposomes, and may be prepared by any method known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds of the invention may be incorporated into slow release formulations.

It will be appreciated that the optimum time course will depend on factors such as the time taken for the peak plasma concentration of the compound to be reached after administration, and the elimination half-life of each compound. Preferably the time difference is less than the half-life of the first component to be administered.

Suitable pharmaceutical compositions and dosage forms may be prepared using conventional methods known to those in the field of pharmaceutical formulation and described in the relevant texts and literature, for example, in Remington: The Science and Practice of Pharmacy (Easton, Pa.: Mack Publishing Co., 1995).

It is especially advantageous to formulate combined preparations of the invention in unit dosage form for ease of administration and uniformity of dosage. The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, for example, two tablets or capsules taken together may provide a therapeutically effective dosage, such that the unit dosage in each tablet or capsule is approximately 50% of the therapeutically effective amount.

Preparations according to the invention for parenteral administration include sterile aqueous and non-aqueous solutions, suspensions, and emulsions. Injectable aqueous solutions contain the active agent in water-soluble form. Examples of non-aqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations may be rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

In addition to the formulations described previously, the active agent may be formulated as a depot preparation for controlled release of the active agent, preferably sustained release over an extended time period. These sustained release dosage forms are generally administered by implantation (for example, subcutaneously or intramuscularly or by intramuscular injection).

Combined preparations of the invention may be packaged with instructions for administration of the components on the combination. The instructions may be recorded on a suitable recording medium or substrate. For example, the instructions may be printed on a substrate, such as paper or plastic. The instructions may be present as a package insert, in the labeling of the container or components thereof (i.e., associated with the packaging or sub-packaging). In other embodiments, the instructions are present as an electronic storage data file present on a suitable computer readable storage medium, for example, CD-ROM, diskette. Some or all components of the combined preparation may be packaged in suitable packaging to maintain sterility.

### Experimental Methods

The following abbreviations have been used:
Aq Aqueous
DCM Dichloromethane
DIPEA Diisopropylethylamine
Ee Enantiomeric excess
ES+ Electrospray
EtOAc Ethyl acetate
H Hour(s)
HPLC High performance liquid chromatography
HRMS High resolution mass spectrometry
LCMS Liquid chromatography mass spectrometry
M Molar
MeOH Methanol
[MH+] Protonated molecular ion
min Minutes
RP Reverse phase
MS Mass spectrometry
R_{T} Retention time
sat Saturated
THF Tetrahydrofuran
TFA Trifluoroacetic acid

All reagents were commercial grade and were used as received without further purification, unless otherwise specified. Reagent grade solvents were used in all cases. Analytical LCMS was performed on a Waters ZQ mass spectrometer connected to an Agilent 1100 HPLC system. Analytical HPLC was performed on an Agilent 1100 system. High-resolution mass spectra (HRMS) were obtained on an Agilent MSD-TOF connected to an Agilent 1100 HPLC system. During the analyses the calibration was checked by two masses and automatically corrected when needed. Spectra are acquired in positive electrospray mode. The acquired mass range was m/z 100-1100. Profile detection of the mass peaks was used. Flash chromatography was performed on either a CombiFlash Companion system equipped with RediSep silica columns or a Flash Master Personal system equipped with Strata SI-1 silica gigatubes. Reverse Phase HPLC was performed on a Gilson system (Gilson 322 pump with Gilson 321 equilibration pump and Gilson 215 autosampler) equipped with Phenomenex Synergi Hydro RP 150×10 mm, YMC ODS-A 100/150×20 mm or Chirobiotic T 250×10 mm columns. Reverse phase column chromatography was performed on a Gilson system (Gilson 321 pump and Gilson FC204 fraction collector) equipped with Merck LiChroprep® RP-18 (40-63 μm) silica columns. The compounds were automatically named using ACD 6.0. All compounds were dried in a vacuum oven overnight.

Analytical HPLC and LCMS Data were Obtained with:

System A: Phenomenex Synergi Hydro RP (C18, 30×4.6 mm, 4 μm), gradient 5-100% CH₃CN (+0.085% TFA) in water (+0.1% TFA), 1.5 mL/min, with a gradient time of 1.75 min, 200 nm, 30° C.; or

System B: Phenomenex Synergi Hydro RP (C18, 150×4.6 mm, 4 μm), gradient 5-100% CH₃CN (+0.085% TFA) in water (+0.1% TFA), 1.5 mL/min with a gradient time of 7 min, 200 nm, 30° C.

Chiral HPLC Data were Obtained with:

System C: Chirobiotic V polar ionic mode (150×4.6 mm), 70% MeOH in 10 mM aq ammonium formate buffer, 1.0 mL/min, over 10 min, 200 nm, 30° C.

### Preparation of 1-(4-{5-[3-(4-Fluorophenyl)-3H-imidazo[4,5-c]pyridin-2-yl]pyridin-2-yl}piperazin-1-yl)ethan-1-one (Compound 2)

### 1-(4-{5-[3-(4-Fluorophenyl)-3H-imidazo[4,5-c]pyridin-2-yl]pyridin-2-yl}piperazin-1-yl)ethan-1-one has the following structure

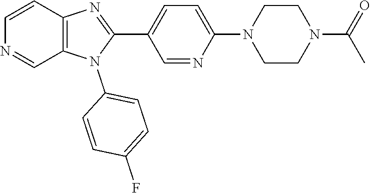 [SMILES: CC(=O)N1CCN(c2ccc(-c3nc4ccncc4n3-c3ccc(F)cc3)cn2)CC1]

This compound is Example 86 of published patent application WO 2014/140592, the synthesis of which compound is described in detail therein.

### Preparation of 1-{5-[3-(4-Fluorophenyl)-3H-imidazo[4,5-c]pyridin-2-yl]pyridin-2-yl}-4-methanesulfonylpiperazine (Compound 3)

### 1-{5-[3-(4-Fluorophenyl)-3H-imidazo[4,5-c]pyridin-2-yl]pyridin-2-yl}-4-methanesulfonylpiperazine has the following structure

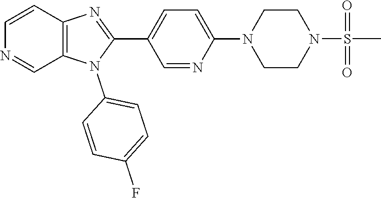 [SMILES: CS(=O)(=O)N1CCN(c2ccc(-c3nc4ccncc4n3-c3ccc(F)cc3)cn2)CC1]

This compound is Example 89 of published patent application WO 2014/140592, the synthesis of which compound is described in detail therein.

### Preparation of 4-{5-[3-(4-Fluorophenyl)-3H-imidazo[4,5-c]pyridin-2-yl]pyridin-2-yl}morpholine (Compound 4)

### 4-{5-[3-(4-Fluorophenyl)-3H-imidazo[4,5-c]pyridin-2-yl]pyridin-2-yl}morpholine has the following structure

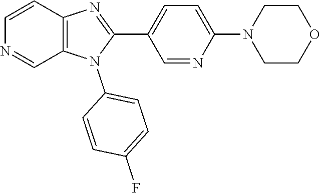 [SMILES: Fc1ccc(-n2c(-c3ccc(N4CCOCC4)nc3)nc3ccncc32)cc1]

This compound is Example 54 of published patent application WO 2014/140592, the synthesis of which compound is described in detail therein.

### Biological Data

The effects of compounds on stress induced allodynia in a rat model of medication overuse headache (MOH) The effects of compounds on stress induced cephalic and extracephalic allodynia in sumatriptan-primed rats was tested.

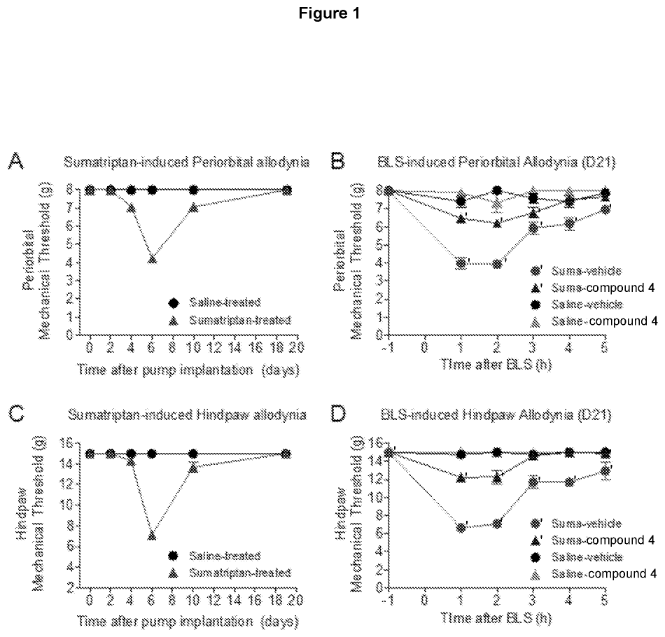

Sprague Dawley rats were implanted with osmotic minipumps (model 2001; Alzet, Cupertino, Calif., USA) providing continuous infusion of sumatriptan (0.6 mg/kg/day, s.c) or vehicle (saline, 0.9% NaCl) for 7 days. Mechanical facial (FIG. 1; A) and hindpaw (FIG. 1; C) allodynia was measured during a 19 day time course.

On day 20, when mechanical thresholds are at basal levels, all rats received either 4-{5-[3-(4-Fluorophenyl)-3H-imidazo[4,5-c]pyridin-2-yl]pyridin-2-yl}morpholine (compound 4) or vehicle orally (33.3 mg/ml, Dose 3 ml/kg) and then they were exposed to bright lights for 1 h (bright light stress; BLS). Rats received a second dose of the assigned treatment in the afternoon.

On day 21 all rats received a third dose of the assigned treatment and then they were again exposed to BLS for 1 h. Mechanical facial (FIG. 1; B) and hindpaw (FIG. 1; D) allodynia was measured over a 5 h time course.

Sumatriptan-treated, but not saline-treated, rats developed generalized allodynia during minipump infusion measured in the periorbital and hindpaw regions (FIG. 1; A, C; day 6). Mechanical thresholds returned to baseline on days 10 and 19. Saline-treated animals do not show any allodynia following the exposure to bright light stress. In contrast, sumatriptan-primed animals treated with vehicle developed time-dependent mechanical allodynia following the exposure to BLS (FIG. 1; B, D).

4-{5-[3-(4-Fluorophenyl)-3H-imidazo[4,5-c]pyridin-2-yl]pyridin-2-yl}morpholine (compound 4) significantly reduced stress-induced periorbital and hindpaw allodynia.

TEV48125, a fully humanised CGRP antibody, is efficacious both in the MOH model and in human clinical trials (Kopruszinski et al, Cephalalgia, 2017, 37(6), 560-70; http://www.tevapharm.com/news/teva_announces_positive_results_for_tev_48125_in_phase_iib_chronic_migraine_study_meeting_primary_and_secondary_endpoints_02_15.aspx)

### CNS Penetration

Central nervous system (CNS) penetration of compounds of the invention can be determined by for example, intravenous dosing in a rat and subsequent quantitative LCMS analysis of the drug concentration in plasma and whole brain homogenate. The total brain:plasma ratio can then be calculated. This total ratio can be adjusted for plasma protein binding (PPB) and brain tissue binding (BTB) determined by standard means to give an unbound (free) brain:plasma ratio. For example, Compound 4 of the invention has an unbound (free) brain:plasma ratio of 0.72, indicating good CNS penetration of the compound.

For the treatment of migraine, it may be particularly advantageous that the compounds penetrate the CNS.

### VAP-1 Inhibition Assay

LJP1207, (S)-carbidopa, 1-(4-{5-[3-(4-Fluorophenyl)-3H-imidazo[4,5-c]pyridin-2-yl]pyridin-2-yl}piperazin-1-yl)ethan-1-one (Compound 2),
1-{5-[3-(4-Fluorophenyl)-3H-imidazo[4,5-c]pyridin-2-yl]pyridin-2-yl}-4-methanesulfonylpiperazine (Compound 3), and
4-{5-[3-(4-Fluorophenyl)-3H-imidazo[4,5-c]pyridin-2-yl]pyridin-2-yl}morpholine (Compound 4) are inhibitors of VAP-1 (see Table 1).

This assay is performed at room temperature with purified recombinantly expressed human VAP-1 (SSAO). Enzyme was prepared essentially as described in Ohman et al. (Protein Expression and Purification 46 (2006) 321-331). The enzyme activity is assayed with benzylamine as substrate by measuring either benzaldehyde production, using 14C-labeled substrate, or by utilizing the production of hydrogen peroxide in a horseradish peroxidise (HRP) coupled reaction. Briefly, test compounds are dissolved in dimethyl sulfoxide (DMSO) to a concentration of 10 mM. Dose-response measurements are assayed by either creating 1:10 serial dilutions in DMSO to produce a 7 point curve or by making 1:3 serial dilutions in DMSO to produce 11 point curves. The top concentrations are adjusted depending on the potency of the compounds and subsequent dilution in reaction buffer yielded a final DMSO concentration ≤2%.

Hydrogen peroxide detection: In a horseradish peroxidise (HRP) coupled reaction, hydrogen peroxide oxidation of 10-acetyl-3,7-dihydroxyphenoxazine produces resorufin, which is a highly fluorescent compound (Zhout and Panchuk-Voloshina. Analytical Biochemistry 253 (1997) 169-174; AmplexR Red Hydrogen Peroxide/peroxidise Assay kit, Invitrogen A22188). Enzyme and compounds in 50 mM sodium phosphate, pH 7.4 are set to pre-incubate in flat-bottomed microtiter plates for approximately 15 minutes before initiating the reaction by addition of a mixture of HRP, benzylamine and Amplex reagent. Benzylamine concentration is fixed at a concentration corresponding to the Michaelis constant, determined using standard procedures. Fluorescence intensity is then measured at several time points during 1-2 hours, exciting at 544 nm and reading the emission at 590 nm. For the human SSAO assay final concentrations of the reagents in the assay wells are: SSAO enzyme 1 mg/ml, benzylamine 100 μM, Amplex reagent 20 μM, HRP 0.1 U/mL and varying concentrations of test compound. The inhibition is measured as % decrease of the signal compared to a control without inhibitor (only diluted DMSO). The background signal from a sample containing no SSAO enzyme is subtracted from all data points. Data is fitted to a four parameter logistic model and IC₅₀ values are calculated, for example by using the GraphPad Prism 4 or XLfit 4 programs.

TABLE 1

| | Human |
|---|---|
| Compound | VAP-1 IC₅₀ |
| LJP1207 | 34 nM |
| (S)-Carbidopa | 142 nM |
| 1-(4-{5-[3-(4-Fluorophenyl)-3H-imidazo[4,5- | 31 nM |
| c]pyridin-2-yl]pyridin-2-yl}piperazin-1-yl)ethan-1- | |
| one (compound 2) | |
| 1-{5-[3-(4-Fluorophenyl)-3H-imidazo[4,5- | 4 nM |
| c]pyridin-2-yl]pyridin-2-yl}-4- | |
| methanesulfonylpiperazine (Compound 3) | |
| 4-{5-[3-(4-Fluorophenyl)-3H-imidazo[4,5- | 13 nm |
| c]pyridin-2-yl]pyridin-2-yl}morpholine (Compound | |
| 4) | |

1. A VAP-1 inhibitor for use in the prevention and/or treatment of migraine.

2. A method for the prevention and/or treatment of migraine, which comprises administering to a subject suffering from migraine an effective amount of a VAP-1 inhibitor.

3. A pharmaceutical composition for use in the prevention and/or treatment of migraine, which comprises a VAP-1 inhibitor; and a pharmaceutically acceptable carrier, excipient, or diluent.

4. The VAP-1 inhibitor for use according to claim 1, wherein the VAP-1 inhibitor is a compound of Formula (III) or a pharmaceutically acceptable salt, or N-oxide thereof 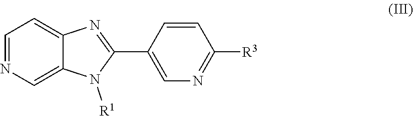
wherein
R¹ is a phenyl ring, or a 5 or 6-membered heteroaryl ring, either ring being optionally substituted with one or more substituents selected from halogen, cyano, C₁₋₄-alkyl, halo-C₁₋₄-alkyl, cyano-C₁₋₄-alkyl, a 3-7 membered cycloalkyl ring, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, and —NR⁶S(O)₂R⁵; wherein
R^{4A}, R^{4B} R⁵ and R⁶ are each independently selected from hydrogen, C₁₋₄-alkyl or halo-C₁₋₄-alkyl, or
R^{4A} and R^{4B} together with the nitrogen to which they are attached form a 3-7-membered cyclic amino group, optionally substituted by one or more substituents selected from: halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₁₋₂, —NHC₁₋₄-alkyl, —NHhalo-C₁₋₄-alkyl;
R³ is a 3-7 membered heterocyclic ring, a 3-7 membered cycloalkyl ring, or a 5 or 6-membered heteroaryl ring, any one of the rings being optionally substituted with one or more substituents selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵.

5. The VAP-1 inhibitor for use according to claim 4, wherein R¹ is a phenyl ring optionally substituted with one or more substituents as defined in claim 4.

6. The VAP-1 inhibitor for use according to claim 4, wherein the VAP-1 inhibitor is a compound of Formula (IIIa) or a pharmaceutically acceptable salt, or N-oxide thereof

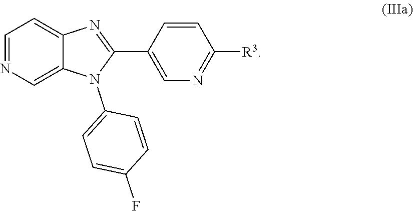

7. The VAP-1 inhibitor for use according to claim 4, wherein R³ is a 3-7 membered heterocyclic ring optionally substituted with one or more substituents as defined in claim 4.

8. The VAP-1 inhibitor for use claim 4, wherein R³ is a piperazine or morpholine ring optionally substituted with one or more substituents as defined in claim 4.

9. The VAP-1 inhibitor for use according to claim 8, wherein piperazine or morpholine ring of R³ is joined to the rest of the molecule through a nitrogen atom of that piperazine or morpholine ring.

10. The VAP-1 inhibitor for use according to claim 4, wherein R³ is a piperazine ring substituted with at least one substituent as defined in claim 4 on a nitrogen atom in that piperazine ring.

11. The VAP-1 inhibitor for use according to claim 4, wherein the VAP-1 inhibitor is selected from the group consisting of
1-(4-{5-[3-(4-Fluorophenyl)-3H-imidazo[4,5-c]pyridin-2-yl]pyridin-2-yl}piperazin-1-yl)ethan-1-one;
4-{5-[3-(4-Fluorophenyl)-3H-imidazo[4,5-c]pyridin-2-yl]pyridin-2-yl}morpholine; and
1-{5-[3-(4-Fluorophenyl)-3H-imidazo[4,5-c]pyridin-2-yl]pyridin-2-yl}-4-methanesulfonylpiperazine.

12. The VAP-1 inhibitor for use according to claim 1, wherein the VAP-1 inhibitor is a compound of Formula (I) or a pharmaceutically acceptable salt, or N-oxide thereof 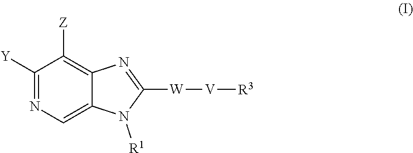
wherein:
Y is selected from hydrogen, hydroxyl, —NH₂, —NH—C₁₋₄-alkyl, —NH-halo-C₁₋₄-alkyl, or —C₁₋₄-alkoxy;
Z is selected from hydrogen, halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₂, —NHC₁₋₄-alkyl, or —NHhalo-C₁₋₄-alkyl;
R¹ is a phenyl ring, or a 5 or 6-membered heteroaryl ring, either ring being optionally substituted with one or more substituents selected from halogen, cyano, C₁₋₄-alkyl, halo-C₁₋₄-alkyl, cyano-C₁₋₄-alkyl, a 3-7 membered cycloalkyl ring, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, and —NR⁶S(O)₂R⁵; wherein
R^{4A}, R^{4B} R⁵ and R⁶ are each independently selected from hydrogen, C₁₋₄-alkyl or halo-C₁₋₄-alkyl, or
R^{4A} and R^{4B} together with the nitrogen to which they are attached form a 3-7 membered cyclic amino group, optionally substituted by one or more substituents selected from: halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₁₋₂, —NHC₁₋₄-alkyl, —NHhalo-C₁₋₄-alkyl;
X is selected from —N═ or —C(R²)═;
R² is selected from hydrogen, halogen, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵;
W is a phenyl ring or a 5 or 6-membered heteroaryl ring, either ring being optionally substituted with one or more substituents selected from halogen, cyano, oxo C₁₋₄-alkyl, halo-C₁₋₄-alkyl, cyano-C₁₋₄-alkyl, —OR⁵, —NR^{7A}R^{7B}, —NR⁶C(O)OR⁵, —NR⁶C(O)R⁵, —NR⁶C(O)NR^{7A}R^{7B}, —C(O)NR^{7A}R^{7B}, —C(O)R⁵, —C(O)OR⁵, —SO₂R⁵, —SO₂NR^{7A}R^{7B} and —NR⁶S(O)₂R⁵;
R^{7A} and R^{7B} are independently hydrogen, C₁₋₄-alkyl or halo-C₁₋₄-alkyl,
V is selected from a bond, —O—, —N(R⁶)—, —(C═O)—, —CONR⁶—, —NR⁶C(O)—, or —C₁₋₄-alkylene-, wherein the C₁₋₄-alkylene group is optionally substituted by halogen, and wherein any one of the carbon atoms of the C₁₋₄-alkylene group may be replaced by —O— or —N(R⁶)—;
R³ is selected from hydrogen, —C₁₋₄-alkyl, —C₁₋₄-alkyl-C₁₋₄-alkoxy or a 3-7 membered heterocyclic ring or 3-7 membered cycloalkyl ring, or a 5 or 6-membered heteroaryl ring, any one of the rings being optionally substituted with one or more substituents selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵;
with the proviso that groups —W—V—R³ and/or R¹ are not 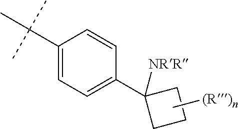
wherein
n is 0, 1, or 2;
R′ and R″ are independently selected from the group consisting of H, —C₁-C₆alkyl, —(C═O)—C₁-C₆ alkyl and —(C═O)OC(CH₃)₃; and
R′″ is H, OH, or C₁-C₆ alkyl.

13. The VAP-1 inhibitor for use according to claim 1, wherein the VAP-1 inhibitor is a compound of Formula (I) or a pharmaceutically acceptable salt, or N-oxide thereof 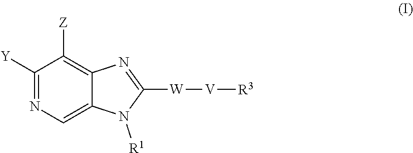
wherein:
Y is selected from hydrogen, hydroxyl, —NH₂, —NH—C₁₋₄-alkyl, —NH-halo-C₁₋₄-alkyl, or —C₁₋₄-alkoxy;
Z is selected from hydrogen, halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₂, —NHC₁₋₄-alkyl, or —NHhalo-C₁₋₄-alkyl;
R¹ is a phenyl ring, or a 5 or 6-membered heteroaryl ring, either ring being optionally substituted with one or more substituents selected from halogen, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, and —NR⁶S(O)₂R⁵; wherein
R^{4A}, R^{4B} R⁵ and R⁶ are each independently selected from hydrogen, C₁₋₄-alkyl or halo-C₁₋₄-alkyl, or
R^{4A} and R^{4B} together with the nitrogen to which they are attached form a 3 to 7-membered cyclic amino group, optionally substituted by one or more substituents selected from: halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₂, —NHC₁₋₄-alkyl, —NHhalo-C₁₋₄-alkyl;
X is selected from —N═ or —C(R²)═;
R² is selected from hydrogen, halogen, cyano, C₁₋₄-alkyl, halo-C₁₋₄-alkyl, cyano-C₁₋₄-alkyl, —OR⁵, —NR^{4A}R^{4B}, —NR⁶C(O)OR, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵;
W is a phenyl ring or a 5 or 6-membered heteroaryl ring, either ring being optionally substituted with one or more substituents selected from halogen, cyano, C₁₋₄-alkyl, halo-C₁₋₄-alkyl, cyano-C₁₋₄-alkyl, —OR⁵, —NR^{7A}R^{7B}, —NR⁶C(O)OR⁵, —NR⁶C(O)R⁵, —NR⁶C(O)NR^{7A}R^{7B}, —C(O)NR^{7A}R^{7B}, —C(O)R⁵, —C(O)OR⁵, —SO₂R⁵, —SO₂NR^{7A}R^{7B} and —NR⁶S(O)₂R⁵;
R^{7A} and R^{7B} are independently hydrogen, C₁₋₄-alkyl or halo-C₁₋₄-alkyl,
V is selected from a bond, —O—, —N(R⁶)—, —(C═O)—, —CONR⁶—, —NR⁶C(O)—, or —C₁₋₄-alkylene-, wherein the C₁₋₄-alkylene group is optionally substituted by halogen, and wherein any one of the carbon atoms of the C₁₋₄-alkylene group may be replaced by —O— or —N(R⁶)—;
R³ is hydrogen or a 3-7 membered heterocyclic ring or 3-7 membered cycloalkyl ring selected from cyclopropyl, cyclopentyl or cyclohexyl, or a 5 or 6-membered heteroaryl ring, any one of the rings being optionally substituted with one or more substituents selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵—, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵.

14. The VAP-1 inhibitor for use according to claim 12, wherein
(i) Y is hydrogen;
(ii) Z is hydrogen;
(iii) R¹ is phenyl or 6-membered heteroaryl, optionally substituted with one or more substituents selected from halogen, C₁₋₄-alkyl or halo-C₁₋₄-alkyl; preferably R¹ is phenyl or pyridyl, optionally substituted with one or more substituents selected from F, Cl or CH₃; and/or
(iv) X is —C(R²)═, and R² is hydrogen, halogen, cyano, C₁₋₄-alkyl, or halo-C₁₋₄-alkyl; preferably R² is hydrogen.

15. The VAP-1 inhibitor for use claim 12, wherein W is
(a) a phenyl ring optionally substituted with one or more substituents as defined in claim 12;
(b) a 6-membered heteroaryl ring selected from pyridine, pyridazine, pyrazine, or pyrimidine optionally substituted with one or more substituents as defined in claim 12;
(c) a 5-membered heteroaryl ring selected from oxazole, thiazole or imidazole optionally substituted with one or more substituents as defined in claim 12; or
(d) an imidazolyl ring optionally substituted as in claim 12, and wherein the imidazolyl ring is connected to the pyrrolopyridine core via an imidazolyl ring carbon atom.

16. The VAP-1 inhibitor for use according to claim 12, wherein W is optionally substituted with one or more substituents selected from fluoro, chloro, cyano, CH₃ or CF₃.

17. The VAP-1 inhibitor for use according to claim 12, wherein
(A) V is —CH₂—, —(CH₂)₂—, or —N(R⁶)CH₂—, or —CH₂—N(R⁶)—, optionally wherein, when dependent on claim 12, R³ is cyclopropyl, cyclobutyl, cyclopentyl or cyclohexyl optionally substituted as defined in claim 12;
(B) R³ is formed from —NR^{4A}R^{4B} wherein R^{4A} and R^{4B}, together with the nitrogen atom to which they are attached join together to form a 4 to 7-membered heterocyclic ring optionally substituted as defined in claim 12; or
(C) R³ is selected from the group consisting of: 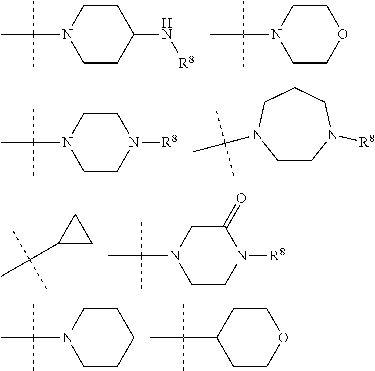
wherein R⁵ is selected from hydrogen, CH₃, —CONH₂, —NHCONH₂, —S(O)₂CH₃, —COCH₃.

18. The VAP-1 inhibitor for use according to claim 12, wherein VAP-1 inhibitor is a compound selected from the group consisting of
3-[3-(4-Chlorophenyl)-3H-imidazo[4,5-c]pyridin-2-yl]pyridine;
4-[3-(4-Chlorophenyl)-3H-imidazo[4,5-c]pyridin-2-yl]pyridine;
4-({5-[3-(4-Chlorophenyl)-3H-imidazo[4,5-c]pyridin-2-yl]pyridin-2-yl}methyl)morpholine;
4-{6-[3-(4-Chlorophenyl)-3H-imidazo[4,5-c]pyridin-2-yl]pyridazin-3-yl}morpholine;
4-{5-[3-(4-Chlorophenyl)-3H-imidazo[4,5-c]pyridin-2-yl]pyrazin-2-yl}morpholine;
4-({5-[3-(4-Chlorophenyl)-3H-imidazo[4,5-c]pyridin-2-yl]pyridin-2-yl}carbonyl)morpholine;
5-[3-(4-Chlorophenyl)-3H-imidazo[4,5-c]pyridin-2-yl]-N-(oxan-4-yl)pyrazin-2-amine;
1-{5-[3-(4-Chlorophenyl)-3H-imidazo[4,5-c]pyridin-2-yl]pyridin-2-yl}piperidin-4-amine;
N-(Cyclopropylmethyl)-5-[3-(4-fluorophenyl)-3H-imidazo[4,5-c]pyridin-2-yl]pyrimidin-2-amine;
N-Cyclopropyl-5-[3-(4-fluorophenyl)-3H-imidazo[4,5-c]pyridin-2-yl]pyrimidin-2-amine;
5-[3-(4-(Chlorophenyl)-3H-imidazo[4,5-c]pyridin-2-yl]-N-(oxan-4-yl)pyrimidin-2-amine;
4-{5-[3-(4-Fluorophenyl)-3H-imidazo[4,5-c]pyridin-2-yl]pyrimidin-2-yl}piperazin-2-one;
4-{5-[3-(4-Chlorophenyl)-3H-imidazo[4,5-c]pyridin-2-yl]pyrimidin-2-yl}piperazin-2-one;
5-[3-(4-Chlorophenyl)-3H-imidazo[4,5-c]pyridin-2-yl]-N-cyclopropylpyridine-2-carboxamide;
3-[3-(4-Fluorophenyl)-3H-imidazo[4,5-c]pyridin-2-yl]-6-(oxan-4-yl)pyridazine;
N-{5-[3-(4-Chlorophenyl)-3H-imidazo[4,5-c]pyridin-2-yl]pyridin-2-yl)}methanesulfonamide;
1-{4-[3-(4-Chlorophenyl)-3H-imidazo[4,5-c]pyridin-2-yl]-1,3-thiazol-2-yl}piperazine;
1-{5-[3-(4-Chlorophenyl)-3H-imidazo[4,5-c]pyridin-2-yl]-1,3-oxazol-2-yl}piperazine;
1-{5-[3-(4-Chlorophenyl)-3H-imidazo[4,5-c]pyridin-2-yl]-1,3-thiazol-2-yl}piperazine;
5-[3-(4-Fluorophenyl)-3H-imidazo[4,5-c]pyridin-2-yl]-N-(oxan-4-yl)pyrimidin-2-amine;
4-{5-[3-(4-Fluorophenyl)-3H-imidazo[4,5-c]pyridin-2-yl]-4-methylpyridin-2-yl}morpholine;
4-{5-[3-(4-Chloro-2-fluorophenyl)-3H-imidazo[4,5-c]pyridin-2-yl]-4-methylpyridin-2-yl}morpholine;
(2R,6S)-4-(5-[3-(4-Fluorophenyl)-3H-imidazo[4,5-c]pyridin-2-yl]pyrimidin-2-yl)-2,6-dimethylmorpholine;
4-{5-[3-(4-Fluorophenyl)-3H-imidazo[4,5-c]pyridin-2-yl]pyrimidin-2-yl}-2,2-dimethylmorpholine;
4-{5-[3-(4-Fluorophenyl)-3H-imidazo[4,5-c]pyridin-2-yl]pyrimidin-2-yl}-1,4-oxazepane;
4-{5-[3-(4-Fluorophenyl)-3H-imidazo[4,5-c]pyridin-2-yl]-4-methylpyrimidin-2-yl}morpholine;
4-{5-[3-(4-Chloro-2-fluorophenyl)-3H-imidazo[4,5-c]pyridin-2-yl]-4-methylpyrimidin-2-yl}morpholine;
4-{5-[3-(4-Fluorophenyl)-3H-imidazo[4,5-c]pyridin-2-yl]-6-methoxypyridin-2-yl}morpholine;
4-{(5-[3-(4-Fluorophenyl)-3H-imidazo[4,5-c]pyridin-2-yl]-4,6-dimethylpyridin-2-yl}morpholine;
2-Cyclopropyl-5-[3-(4-fluorophenyl)-3H-imidazo[4,5-c]pyridin-2-yl]pyrimidine;
5-[3-(4-Chlorophenyl)-3H-imidazo[4,5-c]pyridin-2-yl]pyrimidin-2-amine;
4-[3-(4-Chlorophenyl)-3H-imidazo[4,5-c]pyridin-2-yl]-1-methyl-1,2-dihydropyridin-2-one;
5-[3-(4-Chlorophenyl)-3H-imidazo[4,5-c]pyridin-2-yl]-1-methyl-1,2-dihydropyridin-2-one;
4-[3-(4-Chloro-2-fluorophenyl)-3H-imidazo[4,5-c]pyridin-2-yl]-1,2-dihydropyridin-2-one;
5-[3-(4-Chloro-2-fluorophenyl)-3H-imidazo[4,5-c]pyridin-2-yl]-1,2-dihydropyridin-2-one;
(2R,6S)-2,6-Dimethyl-4-{5-[3-(5-methylpyridin-2-yl)-3H-imidazo[4,5-c]pyridin-2-yl]pyrimidin-2-yl}morpholine;
N-(3-Methoxypropyl)-5-[3-(4-methylphenyl)-3H-imidazo[4,5-c]pyridin-2-yl]pyrimidin-2-amine;
5-[3-(4-Fluorophenyl)-3H-imidazo[4,5-c]pyridin-2-yl]-N-[2-(propan-2-yloxy)ethyl]pyrimidin-2-amine;
5-[3-(4-Methylphenyl)-3H-imidazo[4,5-c]pyridin-2-yl]-N-[2-(propan-2-yloxy)ethyl]pyrimidin-2-amine;
4-{5-[3-(4-Fluorophenyl)-3H-imidazo[4,5-c]pyridin-2-yl]pyrimidin-2-yl}-1-methylpiperazin-2-one;
4-{(5-[3-(2,4-Difluorophenyl)-3H-imidazo[4,5-c]pyridin-2-yl]pyridin-2-yl}morpholine;
N-(2-Ethoxyethyl)-5-[3-(4-fluorophenyl)-3H-imidazo[4,5-c]pyridin-2-yl]pyrimidin-2-amine;
N-(2-Ethoxyethyl)-5-[3-(4-methylphenyl)-3H-imidazo[4,5-c]pyridin-2-yl]pyrimidin-2-amine;
5-[3-(4-Chlorophenyl)-3H-imidazo[4,5-c]pyridin-2-yl]-1H-imidazole;
1-({3-[3-(4-Chlorophenyl)-3H-imidazo[4,5-c]pyridin-2-yl]phenyl}methyl)-4-methylpiperazine;
1-({4-[3-(4-Chlorophenyl)-3H-imidazo[4,5-c]pyridin-2-yl]phenyl}methyl)-4-methylpiperazine;
4-{5-[3-(4-Chlorophenyl)-3H-imidazo[4,5-c]pyridin-2-yl]pyridin-2-yl}morpholine;
1-({4-[3-(4-Chlorophenyl)-3H-imidazo[4,5-c]pyridin-2-yl]phenyl}methyl)-1H-imidazole;
4-({4-[3-(4-Chlorophenyl)-3H-imidazo[4,5-c]pyridin-2-yl]phenyl}methyl)morpholine;
1-{5-[3-(4-Chlorophenyl)-3H-imidazo[4,5-c]pyridin-2-yl]pyridin-2-yl}piperazine;
4-({5-[3-(4-Chlorophenyl)-3H-imidazo[4,5-c]pyridin-2-yl]pyrimidin-2-yl}morpholine;
4-{5-[3-(4-Fluorophenyl)-3H-imidazo[4,5-c]pyridin-2-yl]pyrimidin-2-yl}morpholine;
4-{5-[3-(2-Fluoro-4-methylphenyl)-3H-imidazo[4,5-c]pyridin-2-yl]pyrimidin-2-yl}morpholine;
4-{5-[3-(4-Fluorophenyl)-3H-imidazo[4,5-c]pyridin-2-yl]pyridin-2-yl}morpholine;
4-{5-[3-(4-Fluoro-2-methylphenyl)-3H-imidazo[4,5-c]pyridin-2-yl]pyrimidin-2-yl}morpholine;
4-{5-[3-(2-Chloro-4-fluorophenyl)-3H-imidazo[4,5-c]pyridin-2-yl]pyrimidin-2-yl}morpholine;
4-{5-[3-(4-Methylphenyl)-3H-imidazo[4,5-c]pyridin-2-yl]pyrimidin-2-yl}morpholine;
4-{5-[3-(6-Methylpyridin-3-yl)-3H-imidazo[4,5-c]pyridin-2-yl]pyrimidin-2-yl}morpholine;
4-{5-[3-(4-Bromophenyl)-3H-imidazo[4,5-c]pyridin-2-yl]pyrimidin-2-yl}morpholine;
4-{5-[3-(2-Fluorophenyl)-3H-imidazo[4,5-c]pyridin-2-yl]pyrimidin-2-yl}morpholine;
4-{5-[3-(2-Chloro-4-fluorophenyl)-3H-imidazo[4,5-c]pyridin-2-yl]pyridin-2-yl}morpholine;
4-{5-[3-(4-Fluoro-2-methylphenyl)-3H-imidazo[4,5-c]pyridin-2-yl]pyridin-2-yl}morpholine;
4-{5-[3-(4-Methylphenyl)-3H-imidazo[4,5-c]pyridin-2-yl]pyridin-2-yl}morpholine;
4-{5-[3-(6-Methylpyridin-3-yl)-3H-imidazo[4,5-c]pyridin-2-yl]pyridin-2-yl}morpholine;
4-{2-[6-(Morpholin-4-yl)pyridin-3-yl]-3H-imidazo[4,5-c]pyridin-3-yl}phenol;
4-(5-{3-[4-(Trifluoromethyl)phenyl]-3H-imidazo[4,5-c]pyridin-2-yl}pyridin-2-yl)morpholine;
4-{5-[3-(2-Fluoro-4-methylphenyl)-3H-imidazo[4,5-c]pyridin-2-yl]pyridin-2-yl}morpholine;
4-{5-[3-(2-Fluorophenyl)-3H-imidazo[4,5-c]pyridin-2-yl]pyridin-2-yl}morpholine;
5-[3-(4-Fluorophenyl)-3H-imidazo[4,5-c]pyridin-2-yl]-2-(pyrrolidin-1-yl)pyrimidine;
4-{5-[3-(4-Fluorophenyl)-3H-imidazo[4,5-c]pyridin-2-yl]pyridin-2-yl}-2-methylmorpholine;
5-[3-(4-Fluorophenyl)-3H-imidazo[4,5-c]pyridin-2-yl]-N,N-dimethylpyridin-2-amine;
5-[3-(4-Chlorophenyl)-3H-imidazo[4,5-c]pyridin-2-yl]-N,N-dimethylpyrimidin-2-amine;
4-{4-[3-(4-Fluorophenyl)-3H-imidazo[4,5-c]pyridin-2-yl]pyridin-2-yl}morpholine;
4-{5-[3-(4-Chloro-3-fluorophenyl)-3H-imidazo[4,5-c]pyridin-2-yl]pyrimidin-2-yl}morpholine;
4-{5-[3-(5-Chloropyridin-2-yl)-3H-imidazo[4,5-c]pyridin-2-yl]pyrimidin-2-yl}morpholine;
4-{5-[3-(5-Fluoropyridin-2-yl)-3H-imidazo[4,5-c]pyridin-2-yl]pyrimidin-2-yl}morpholine;
4-{5-[3-(4-Chloro-2-fluorophenyl)-3H-imidazo[4,5-c]pyridin-2-yl]pyrimidin-2-yl}morpholine;
4-{5-[3-(2,4-Difluorophenyl)-3H-imidazo[4,5-c]pyridin-2-yl]pyrimidin-2-yl}morpholine;
4-{5-[3-(5-Methylpyridin-2-yl)-3H-imidazo[4,5-c]pyridin-2-yl]pyrimidin-2-yl}morpholine;
4-{5-[3-(4-Chloro-2-fluorophenyl)-3H-imidazo[4,5-c]pyridin-2-yl]pyridin-2-yl}morpholine;
4-{5-[3-(5-Chloropyridin-2-yl)-3H-imidazo[4,5-c]pyridin-2-yl]pyridin-2-yl}morpholine;
4-{5-[3-(5-Methylpyridin-2-yl)-3H-imidazo[4,5-c]pyridin-2-yl]pyridin-2-yl}morpholine;
5-[3-(4-Chloro-2-fluorophenyl)-3H-imidazo[4,5-c]pyridin-2-yl]-2-(pyrrolidin-1-yl)pyrimidine;
5-[3-(4-Chloro-2-fluorophenyl)-3H-imidazo[4,5-c]pyridin-2-yl]-N,N-dimethylpyrimidin-2-amine;
N-(1-{5-[3-(4-Chlorophenyl)-3H-imidazo[4,5-c]pyridin-2-yl]pyridin-2-yl}piperidin-4-yl)acetamide;
1-(4-{5-[3-(4-Fluorophenyl)-3H-imidazo[4,5-c]pyridin-2-yl]pyridin-2-yl}piperazin-1-yl)ethan-1-one;
1-(4-{5-[3-(4-Fluorophenyl)-3H-imidazo[4,5-c]pyridin-2-yl]pyridin-2-yl}-1,4-diazepan-1-yl)ethan-1-one bis(trifluoroacetic acid);
N-(1-{5-[3-(4-Chlorophenyl)-3H-imidazo[4,5-c]pyridin-2-yl]pyridin-2-yl}piperidin-4-yl)methanesulfonamide;
1-{-[3-(4-Fluorophenyl)-3H-imidazo[4,5-c]pyridin-2-yl]pyridin-2-yl}-4-methanesulfonylpiperazine;
4-{5-[3-(4-Chlorophenyl)-3H-imidazo[4,5-c]pyridin-2-yl]pyridin-2-yl}piperazine-1-carboxamide;
(1-{5-[3-(4-Chlorophenyl)-3H-imidazo[4,5-c]pyridin-2-yl]pyridin-2-yl}piperidin-4-yl)urea;
4-{5-[3-(4-Fluorophenyl)-3H-imidazo[4,5-c]pyridin-2-yl]pyridin-2-yl}piperazine-1-carboxamide;
4-{5-[3-(4-Chlorophenyl)-3H-imidazo[4,5-c]pyridin-2-yl]-1,3-oxazol-2-yl}piperazine-1-carboxamide;
4-{5-[3-(4-Fluorophenyl)-3H-imidazo[4,5-c]pyridin-2-yl]pyridin-2-yl}-1,4-diazepane-1-carboxamide;
4-(5-{3-Phenyl-3H-imidazo[4,5-c]pyridin-2-yl}pyrimidin-2-yl)morpholine;
4-{5-[3-(4-Cyclopropylphenyl)-3H-imidazo[4,5-c]pyridin-2-yl]pyrimidin-2-yl}morpholine;
4-{4-Methyl-5-[3-(4-methylphenyl)-3H-imidazo[4,5-c]pyridin-2-yl]pyridin-2-yl}morpholine;
4-{3-Fluoro-5-[3-(4-fluorophenyl)-3H-imidazo[4,5-c]pyridin-2-yl]pyridin-2-yl}morpholine;
5-[3-(4-Fluorophenyl)-3H-imidazo[4,5-c]pyridin-2-yl]-2-(morpholin-4-yl)-1,4-dihydropyridin-4-one;
5-[3-(4-Fluorophenyl)-3H-imidazo[4,5-c]pyridin-2-yl]-4-methyl-N-(oxan-4-yl)pyridin-2-amine;
N-(Cyclopropylmethyl)-5-[3-(4-fluorophenyl)-3H-imidazo[4,5-c]pyridin-2-yl]-4-methylpyridin-2-amine;
5-[3-(4-Fluorophenyl)-3H-imidazo[4,5-c]pyridin-2-yl]-4-methyl-2-(1H-pyrazol-1-yl)pyridine;
(2R,6S)-2,6-Dimethyl-4-{5-[3-(6-methylpyridin-3-yl)-3H-imidazo[4,5-c]pyridin-2-yl]pyridin-2-yl}morpholine;
(2R,6S)-2,6-Dimethyl-4-{5-[3-(5-methylpyridin-2-yl)-3H-imidazo[4,5-c]pyridin-2-yl]pyridin-2-yl}morpholine;
5-[3-(4-(Chloro-2-fluorophenyl)-3H-imidazo[4,5-c]pyridin-2-yl]pyridin-2-amine;
4-{5-[3-(4-Fluorophenyl)-3H-imidazo[4,5-c]pyridin-2-yl]pyridin-2-yl}-1-methylpiperazin-2-one;
4-{4-Methyl-5-[3-(6-methylpyridin-3-yl)-3H-imidazo[4,5-c]pyridin-2-yl]pyridin-2-yl}morpholine;
4-{5-[3-(4-Fluorophenyl)-3H-imidazo[4,5-c]pyridin-2-yl]-6-methylpyridin-2-yl}morpholine;
4-[3-(4-Fluorophenyl)-3H-imidazo[4,5-c]pyridin-2-yl]-N,N-dimethtylpyridin-2-amine;
5-[3-(4-Chlorophenyl)-3H-imidazo[4,5-c]pyridin-2-yl]pyridin-2-amine;
5-[3-(4-Fluorophenyl)-3H-imidazo[4,5-c]pyridin-2-yl]-N,N,4-trimethylpyridin-2-amine;
5-[3-(4-Fluorophenyl)-3H-imidazo[4,5-c]pyridin-2-yl]-2-(oxolan-3-yloxy)pyridine;
5-[3-(4-Fluorophenyl)-3H-imidazo[4,5-c]pyridin-2-yl]-2-(oxan-4-yloxy)pyridine;
4-[3-(4-Fluorophenyl)-3H-imidazo[4,5-c]pyridin-2-yl]-1-methyl-1,2-dihydropyridin-2-one;
1-Cyclopropyl-4-[3-(4-fluorophenyl)-3H-imidazo[4,5-c]pyridin-2-yl]-1,2-dihydropyridin-2-one;
4-[3-(4-Chloro-2-fluorophenyl)-3H-imidazo[4,5-c]pyridin-2-yl]-1-cyclopropyl-1,2-dihydropyridin-2-one;
N-(2-Methoxyethyl)-N-methyl-5-[3-(4-methylphenyl)-3H-imidazo[4,5-c]pyridin-2-yl]pyrimidin-2-amine;
(2R,6S)-2,6-Dimethyl-4-{5-[3-(6-methylpyridin-3-yl)-3H-imidazo[4,5-c]pyridin-2-yl]pyrimidin-2-yl}morpholine;
5-[3-(4-Methylphenyl)-3H-imidazo[4,5-c]pyridin-2-yl]-N-(oxan-4-yl)pyrimidin-2-amine;
4-[1-(4-Chlorophenyl)-1H-pyrrolo[2,3-c]pyridin-2-yl]pyridine;
2-[1-(4-Chlorophenyl)-1H-pyrrolo[2,3-c]pyridin-2-yl]pyridine;
3-[1-(4-(Chlorophenyl)-1H-pyrrolo[2,3-c]pyridin-2-yl]pyridine;
5-[1-(4-Chlorophenyl)-1H-pyrrolo[2,3-c]pyridin-2-yl]pyrimidin;
2-[1-(4-Chlorophenyl)-1H-pyrrolo[2,3-c]pyridin-2-yl]pyrazine;
1-({4-[1-(4-Chlorophenyl)-1H-pyrrolo[2,3-c]pyridin-2-yl]phenyl}carbonyl)-4-methylpiperazine;
5-[1-(4-Chlorophenyl)-1H-pyrrolo[2,3-c]pyridin-2-yl]-2,4-dimenthyl-1H-imidazole;
4-{5-[1-(4-Chlorophenyl)-1H-pyrrolo[2,3-c]pyridin-2-yl]pyrimidin-2-yl}morpholine;
4-{5-[1-(4-Chlorophenyl)-1H-pyrrolo[2,3-c]pyridin-2-yl]pyrimidin-2-yl}piperazin-2-one;
4-{5-[1-(4-Chlorophenyl)-1H-pyrrolo[2,3-c]pyridin-2-yl]-4-methylpyridin-2-yl}morpholine;
4-{5-[1-(4-Methylphenyl)-1H-pyrrolo[2,3-c]pyridin-2-yl]pyrimidin-2-yl)}morpholine;
4-{5-{1-Phenyl-1H-pyrrolo[2,3-c]pyridin-2-yl}pyrimidin-2-yl}morpholine;
4-{5-[1-(5-Methylpyridin-2-yl)-1H-pyrrolo[2,3-c]pyridin-2-yl]pyrimidin-2-yl}morpholine;
4-{5-[1-(4-Bromophenyl)-1H-pyrrolo[2,3-c]pyridin-2-yl]pyrimidin-2-yl}morpholine;
5-[1-(4-Chlorophenyl)-1H-pyrrolo[2,3-c]pyridin-2-yl]-1-methyl-1H-pyrazole;
4-[1-(4-Chlorophenyl)-1H-pyrrolo[2,3-c]pyridin-2-yl]-1-methyl-1H-pyrazole;
5-[1-(4-(Chlorophenyl)-1H-pyrrolo[2,3-c]pyridin-2-yl]-1-methyl-1H-imidazole;
5-[1-(4-Chlorophenyl)-1H-pyrrolo[2,3-c]pyridin-2-yl]-N,N-dimethylpyrimidin-2-amine;
4-[1-(4-Chlorophenyl)-1H-pyrrolo[2,3-c]pyridin-2-yl]-1-cyclopropyl-1,2-dihydropyridin-2-one;
5-[1-(4-Chlorophenyl)-1H-pyrrolo[2,3-c]pyridin-2-yl]-N-(oxan-4-yl)pyrimidin-2-amine;
4-({5-[1-(4-Chlorophenyl)-1H-pyrrolo[2,3-c]pyridin-2-yl]pyridin-2-yl}methyl)morpholine;
5-[1-(4-Chlorophenyl)-1H-pyrrolo[2,3-c]pyridin-2-yl]-4-methylpyridin-2-amine;
4-[1-(4-Chlorophenyl)-1H-pyrrolo[2,3-c]pyridin-2-yl]-1,2-dihydropyridin-2-one;
4-[1-(4-Chlorophenyl)-1H-pyrrolo[2,3-c]pyridin-2-yl]-1-methyl-1,2-dihydropyridin-2-one;
4-[1-(4-Chlorophenyl)-1H-pyrrolo[2,3-c]pyridin-2-yl]-1-ethyl-1,2-dihydropyridin-2-one;
6-[1-(4-Chlorophenyl)-1H-pyrrolo[2,3-c]pyridin-2-yl]-1-methyl-1,2-dihydropyridin-2-one;
5-[1-(4-Chlorophenyl)-1H-pyrrolo[2,3-c]pyridin-2-yl]-2,3-dihydropyridazin-3-one;
4-[1-(4-Chlorophenyl)-1H-pyrrolo[2,3-c]pyridin-2-yl]pyridin-2-amine;
3-[1-(4-Chlorophenyl)-1H-pyrrolo[2,3-c]pyridin-2-yl]-5-fluoropyridine;
5-[1-(4-Chlorophenyl)-1H-pyrrolo[2,3-c]pyridin-2-yl]-N-(cyclopropylmethyl)pyrimidin-2-amine;
3-Chloro-5-[1-(4-chlorophenyl)-1H-pyrrolo[2,3-c]pyridin-2-yl]pyridine;
5-[1-(4-Chlorophenyl)-1H-pyrrolo[2,3-c]pyridin-2-yl]-2-(1H-pyrazol-1-yl)pyridine;
4-[1-(4-Chlorophenyl)-1H-pyrrolo[2,3-c]pyridin-2-yl]-3-fluoropyridine;
3-Chloro-4-[1-(4-chlorophenyl)-1H-pyrrolo[2,3-c]pyridin-2-yl]pyridine;
4-[1-(4-Chlorophenyl)-1H-pyrrolo[2,3-c]pyridin-2-yl]-3-methylpyridine; and
1-Cyclopropyl-4-{1-phenyl-1H-pyrrolo[2,3-c]pyridin-2-yl}-1,2-dihydropyridin-2-one,
or a pharmaceutically acceptable salt, or N-oxide thereof.

19. The VAP-1 inhibitor for use according to claim 1, wherein the VAP-1 inhibitor is a compound of Formula (II) or a pharmaceutically acceptable salt, or N-oxide thereof 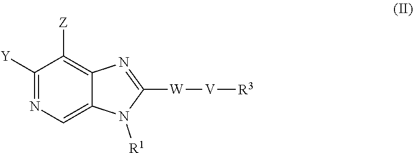
wherein:
Y is selected from hydrogen, hydroxyl, —NH₂, —NH—C₁₋₄-alkyl, —NH-halo-C₁₋₄-alkyl, or —C₁₋₄-alkoxy;
Z is selected from hydrogen, halogen, hydroxyl, cyano, C₁₋₄-alkyl, halo-C₁₋₄-alkyl, C₁₋₄-alkoxy, halo-C₁₋₄-alkoxy, —CONH₂, —SO₂NH₂, —NH₂, —NHC₁₋₄-alkyl, or —NHhalo-C₁₋₄-alkyl;
R¹ is a phenyl ring, or a 5 or 6-membered heteroaryl ring, either ring optionally substituted with one or more substituents selected from halogen, cyano, C₁₋₄-alkyl, halo-C₁₋₄-alkyl, cyano-C₁₋₄-alkyl, —OR⁵, NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, and —NR⁶S(O)₂R⁵; wherein
R^{4A}, R^{4B} R⁵ and R⁶ are each independently selected from hydrogen, C₁₋₄-alkyl or halo-C₁₋₄-alkyl, or
R^{4A} and R^{4B} together with the nitrogen to which they are attached form a 3-7 membered cyclic amino group, optionally substituted by one or more substituents selected from: halogen, hydroxyl, cyano, C₁₋₄-alkyl, C₁₋₄-alkyl, halo-C₁₋₄-alkyl, C₁₋₄-alkoxy, halo-C₁₋₄-alkoxy, —CONH₂, —SO₂NH₂, —NH₂, —NHC₁₋₄-alkyl, —NHhalo-C₁₋₄-alkyl;
R^{7A} and R^{7B} are independently hydrogen, C₁₋₄-alkyl or halo-C₁₋₄-alkyl; and wherein
the group —WVR³ is selected from any one of groups (i)-(iv):
(i) W is a [6,5], [5.6], or [6,6] heteroaryl ring system comprising a phenyl ring or a 6-membered heteroaryl ring fused to a 5 or 6-membered heteroaryl or heterocyclic ring, the fused ring system being optionally substituted on either or both rings with one or more groups selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵, and
V is a direct bond, and
R³ is hydrogen;
(ii) W is a phenyl ring or a 5 or 6-membered heteroaryl ring, either ring optionally substituted with one or more groups selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵, and
V is —NR⁶—, and
R³ is a C₁₋₆-alkyl group substituted with one or more substituents selected from the group consisting of: halogen, hydroxyl, cyano, oxo, and NR^{7A}R^{7B};
(iii) W is a 5 or 6-membered heterocyclic ring optionally substituted with one or more substituents selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵,
V is a direct bond, and
R³ is a phenyl ring or a 5 or 6-membered heteroaryl ring optionally substituted with one or more substituents selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵;
(iv) W is a direct bond, V is a group selected from **—(C═O)—(CH₂)—, —CONR⁶ (CH₂)—, **—NR⁶C(O)—(CH₂)ₙ—, **—NR⁶C(O)O—(CH₂)ₙ— wherein the bond marked ** is connected to the rest of the molecule, or —C₁₋₄-alkylene-, wherein any one of the the —(CH₂)— groups, including the C₁₋₄-alkylene group, group is optionally substituted by halogen, and wherein any one of the carbon atoms of the C₁₋₄-alkylene group may be replaced by —O— or —N(R⁶)—, and
n is 0, 1, 2, 3, or 4
R³ is selected from:
a C₁₋₆-alkyl group optionally substituted with one or more substituents selected from the group consisting of: halogen, hydroxyl, cyano, oxo, C₁₋₄ alkoxy, C₁₋₄alkoxy and NR^{7A}R^{7B}; or
a 3-7 membered heterocyclic or cycloalkyl ring, a phenyl ring, or a 5 or 6-membered heteroaryl ring, any of which rings is optionally substituted with a group selected from halogen, oxo, hydroxyl, cyano, C₁₋₄-alkyl, halo-C₁₋₄-alkyl, cyano-C₁₋₄-alkyl, —OR⁵, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, —SO₂R⁵, —SO₂NR^{4A}R^{4B} and —NR⁶S(O)₂R⁵.

20. The VAP-1 inhibitor for use according to claim 18, wherein
(i) —WVR³ is as defined in group (i) wherein W is a [6,5] heteroaryl ring system formed by fusing together phenyl and pyrrolidinyl or imidazolyl and wherein either ring is optionally substituted as set out in claim 18, preferably wherein W has the formula A1 or A2: 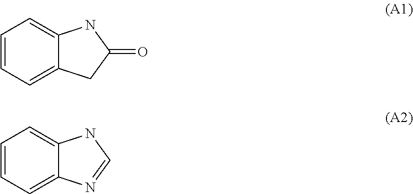
wherein W is optionally substituted on either ring as set out in claim 18, and wherein W is directly connected to the rest of the molecule via a carbon atom on the phenyl ring;
(ii) —WVR³ is as defined in group (ii), and R³ is C₁₋₆-alkyl substituted with one or more groups selected from fluoro, chloro, hydroxyl and C₁₋₄alkyl;
(iii) —WVR³ is as defined in group (ii), and R³ is —CH₂C(CH₃)₂OH;
(iv) —WVR³ is as defined in group (iii), and W is a ring selected from piperidine, morpholine, pyrrolidine, and piperazine, any of which is optionally substituted as set out in claim 18, preferably wherein —WVR³ is 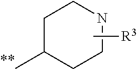
wherein the bond marked ** is directly connected to the rest of the molecule; or
(v) —WVR³ is as defined in group (iv), wherein V is selected from any one of —CONR⁶—, —CONR⁶—(CH₂)—, NR⁶C(O)—, —NR⁶C(O)—(CH₂)—, —NR⁶C(O)O—, —NR⁶C(O)O—(CH₂)—, —(CH₂)—, —(C(H₂)₂—, and —(CH₂)₃—, and/or wherein R³ is a group selected from phenyl, imidazolyl, tetrahydropyranyl, piperidinyl, and piperazinyl, and one of which rings is optionally substituted according to claim 18.

21. The VAP-1 inhibitor for use according to claim 19, wherein
(A) Y is hydrogen;
(B) Z is hydrogen; and/or
(C) R⁶ is hydrogen.

22. The VAP-1 inhibitor for use according to claim 19, wherein the VAP-1 inhibitor is selected from the group consisting of
5-[3-(4-Fluorophenyl)-3H-imidazo[4,5-c]pyridin-2-yl]-2,3-dihydro-1H-indol-2-one;
5-[3-(4-Fluorophenyl)-3H-imidazo[4,5-c]pyridin-2-yl]-1H-1,3-benzodiazole;
1-({5-[3-(4-Fluorophenyl)-3H-imidazo[4,5-c]pyridin-2-yl]pyrimidin-2-yl}amino)-2-methylpropan-2-ol;
2-Methyl-1-({5-[3-(4-methylphenyl)-3H-imidazo[4,5-c]pyridin-2-yl]pyrimidin-2-yl}amino)propan-2-ol;
4-{4-[3-(4-Chlorophenyl)-3H-imidazo[4,5-c]pyridin-2-yl]piperidin-1-yl}pyridine; bis(formic acid);
6-{4-[3-(4-Chlorophenyl)-3H-imidazo[4,5-c]pyridin-2-yl]piperidin-1-yl}-3,4-dihydropyrimidin-4-one;
3-{[3-(4-Chlorophenyl)-3H-imidazo[4,5-c]pyridin-2-yl]methyl}pyridine;
1-{3-[3-(4-chlorophenyl)-3H-imidazo[4,5-c]pyridin-2-yl]propyl}-1H-imidazole;
3-(4-Fluorophenyl)-N-(oxan-4-ylmethyl)-3H-imidazo[4,5-c]pyridine-2-carboxamide
or a pharmaceutically acceptable salt, or N-oxide thereof.

23. The VAP-1 inhibitor for use according to claim 1, wherein the VAP-1 inhibitor is selected from the group consisting of
4-{5-[3-(5-Fluoropyridin-2-yl)-3H-imidazo[4,5-c]pyridin-2-yl]pyridin-2-yl}morpholine;
4-{5-[3-(2,4-Difluorophenyl)-3H-imidazo[4,5-c]pyridin-2-yl]-4-methylpyridin-2-yl}morpholine;
5-[3-(2,4-Difluorophenyl)-3H-imidazo[4,5-c]pyridin-2-yl]-N-(oxan-4-yl)pyrimidin-2-amine;
N,N-Diethyl-5-[3-(6-methylpyridin-3-yl)-3H-imidazo[4,5-c]pyridin-2-yl]pyrimidin-2-amine;
N,N-Diethyl-5-[3-(4-fluorophenyl)-3H-imidazo[4,5-c]pyridin-2-yl]pyrimidin-2-amine;
N,N-Diethyl-5-[3-(4-methylphenyl)-3H-imidazo[4,5-c]pyridin-2-yl]pyrimidin-2-amine;
N,N-Diethyl-5-[3-(5-methylpyridin-2-yl)-3H-imidazo[4,5-c]pyridin-2-yl]pyrimidin-2-amine;
4-{5-[3-(2-Fluoro-4-methylphenyl)-3H-imidazo[4,5-c]pyridin-2-yl]-4-methylpyridin-2-yl}morpholine;
4-{5-[3-(4-Chlorophenyl)-3H-imidazo[4,5-c]pyridin-2-yl]-4-methylpyridin-2-yl}morpholine;
5-[3-(4-Methylphenyl)-3H-imidazo[4,5-c]pyridin-2-yl]-N-(oxan-4-yl)pyridin-2-amine;
2-(4,4-Difluoropiperidin-1-yl)-5-[3-(4-methylphenyl)-3H-imidazo[4,5-c]pyridin-2-yl]pyridine;
4-{5-[3-(5-Chloropyridin-2-yl)-3H-imidazo[4,5-c]pyridin-2-yl]-4-methylpyridin-2-yl}morpholine;
4-{4-Methyl-5-[3-(5-methylpyridin-2-yl)-3H-imidazo[4,5-c]pyridin-2-yl]pyridin-2-yl}morpholine;
4-{5-[3-(5-Fluoropyridin-2-yl)-3H-imidazo[4,5-c]pyridin-2-yl]-4-methylpyridin-2-yl}morpholine;
5-[3-(4-Fluorophenyl)-3H-imidazo[4,5-c]pyridin-2-yl]-N-(oxan-4-yl)pyridin-2-amine;
4-{5-[3-(4-Fluorophenyl)-3H-imidazo[4,5-c]pyridin-2-yl]pyridin-2-yl}thiomorpholine;
N-Cyclopropyl-5-[3-(4-methylphenyl)-3H-imidazo[4,5-c]pyridin-2-yl]pyridin-2-amine;
5-[3-(6-Methylpyridin-3-yl)-3H-imidazo[4,5-c]pyridin-2-yl]-2-(pyrrolidin-1-yl)pyridine;
2-(4-Fluoropiperidin-1-yl)-5-[3-(6-methylpyridin-3-yl)-3H-imidazo[4,5-c]pyridin-2-yl]pyridine;
5-[3-(4-Fluorophenyl)-3H-imidazo[4,5-c]pyridin-2-yl]-N-[2-(morpholin-4-yl)ethyl]pyridin-2-amine;
5-[3-(4-Methylphenyl)-3H-imidazo[4,5-c]pyridin-2-yl]-N-[2-(morpholin-4-yl)ethyl]pyridin-2-amine;
N-Cyclopropyl-5-[3-(4-fluorophenyl)-3H-imidazo[4,5-c]pyridin-2-yl]pyridin-2-amine;
N-Cyclopropyl-5-[3-(6-methylpyridin-3-yl)-3H-imidazo[4,5-c]pyridin-2-yl]pyridin-2-amine;
5-[3-(4-Methylphenyl)-3H-imidazo[4,5-c]pyridin-2-yl]-N-(propan-2-yl)pyridin-2-amine;
5-[3-(6-Methylpyridin-3-yl)-3H-imidazo[4,5-c]pyridin-2-yl]-2-(pyrrolidin-1-yl)pyrimidine;
5-[3-(5-Methylpyridin-2-yl)-3H-imidazo[4,5-c]pyridin-2-yl]-2-(pyrrolidin-1-yl)pyrimidine;
5-[3-(5-Fluoropyridin-2-yl)-3H-imidazo[4,5-c]pyridin-2-yl]-2-(pyrrolidin-1-yl)pyrimidine;
4-{4-[3-(6-Methylpyridin-3-yl)-3H-imidazo[4,5-c]pyridin-2-yl]phenyl}morpholine;
5-[3-(4-Methylphenyl)-3H-imidazo[4,5-c]pyridin-2-yl]-2-(pyrrolidin-1-yl)pyrimidine;
4-{4-[3-(5-Methylpyridin-2-yl)-3H-imidazo[4,5-c]pyridin-2-yl]phenyl}morpholine;
2-Methyl-5-{2-[4-(pyrrolidin-1-yl)phenyl]-3H-imidazo[4,5-c]pyridin-3-yl}pyridine;
5-{2-[2-Fluoro-4-(pyrrolidin-1-yl)phenyl]-3H-imidazo[4,5-c]pyridin-3-yl}-2-methylpyridine;
4-{3-Fluoro-4-[3-(6-methylpyridin-3-yl)-3H-imidazo[4,5-c]pyridin-2-yl]phenyl}morpholine;
5-{2-[3-Fluoro-4-(pyrrolidin-1-yl)phenyl]-3H-imidazo[4,5-c]pyridin-3-yl}-2-methylpyridine;
N-{4-[3-(6-Methylpyridin-3-yl)-3H-imidazo[4,5-c]pyridin-2-yl]phenyl)}oxan-4-amine;
5-Methyl-2-{2-[4-(pyrrolidin-1-yl)phenyl]-3H-imidazo[4,5-c]pyridin-3-yl}pyridine;
5-{2-[4-(4-Fluoropiperidin-1-yl)phenyl]-3H-imidazo[4,5-c]pyridin-3-yl}-2-methylpyridine;
2-Chloro-5-[3-(4-chlorophenyl)-3H-imidazo[4,5-c]pyridin-2-yl]pyridine
2-Chloro-5-[3-(4-fluorophenyl)-3H-imidazo[4,5-c]pyridin-2-yl]pyridine
or a pharmaceutically acceptable salt, or N-oxide thereof.

24. The VAP-1 inhibitor for use according to claim 1, wherein the VAP-1 inhibitor is selected from the group consisting of (S)-carbidopa, benserazide, LJP1207, LJP1586, mofegiline, BTT1023, RTU-1096, PXS4728 and ASP8232 or a hydrate or pharmaceutically acceptable salt thereof.

25. The VAP-1 inhibitor for use according to claim 1, wherein the VAP-1 inhibitor is (S)-carbidopa.

26. The VAP-1 inhibitor for use according to claim 1, wherein the VAP-1 inhibitor has the structure of any one of the specific VAP-1 inhibitor compounds, polypeptides or proteins disclosed herein.

27. The VAP-1 inhibitor for use claim 1, wherein migraine is selected from the group consisting of headache, chronic migraine; episodic migraine; medication overuse headache disorder (MOU); migraine without aura; migraine with aura; migraine aura without headache; ocular migraine; vestibular migraine; basilar migraine; hemiplegic migraine; ophthalmoplegic migraine; and tension-type headache (TTH).

28. The VAP-1 inhibitor for use according to claim 27, wherein migraine is medication overuse headache disorder (MOU).

29. The VAP-1 inhibitor for use according to claim 1, wherein the use or method is for the prevention of migraine.

30. The VAP-1 inhibitor for use according to claim 1, wherein the use or method is for the treatment of migraine.
